(19) United States
(12) Patent Application Publication
(10) Pub. No.: US 2023/0304067 A1
(43) Pub. Date: Sep. 28, 2023

(54) METHODS FOR PREDICTING THE RESPONSE OF A PATIENT TO TREATMENT WITH A PD-1 OR PD-L1 IMMUNE CHECKPOINT INHIBITOR

(71) Applicant: PamGene B.V., HH 's-Hertogenbosch (NL)

(72) Inventor: Richard DE WIJN, Nijmegen (NL)

(73) Assignee: PamGene B.V., HH 's-Hertogenbosch (NL)

(21) Appl. No.: 18/020,330

(22) PCT Filed: Sep. 3, 2021
(86) PCT No.: PCT/EP2021/074319

§ 371 Date: Feb. 8, 2023

(30) Foreign Application Priority Data
Sep. 4, 2020 (EP) 20194616.7

(51) Int. Cl. C12Q 1/48

(52) U.S. Cl. CPC C12Q 1/485; G01N 2333/9121

(57) ABSTRACT

The present invention provides methods for predicting the response of a patient in need of an immune checkpoint inhibitor (ICI), to treatment with a programmed cell death-1 (PD-1) or programmed death-ligand 1 (PD-L1) ICI, comprising the steps of: (a) measuring the kinase activity of at least two kinases independently selected from within at least two families of kinases selected from the group consisting of: the VEGFR or PDGFR family of kinases; the SRC family of kinases; the SYK family of kinases; the TAM family of kinases; the JakA family of kinases; and the ALK family of kinases, in a blood sample obtained from said patient, thereby providing a kinase activity profile of said blood sample; and (b) predicting from said kinase activity profile the response of said patient to treatment with said PD-1 or PD-L1 ICI, and kits for predicting the response of a patient in need of an ICI to treatment with a PD-1 or PD-L1 ICI.

### FIELD OF THE INVENTION

The present invention relates to methods and devices for determining or predicting the response of a patient in need of an immune checkpoint inhibitor (ICI) to specific medicaments, more particularly programmed cell death-1 (PD-1) or programmed death-ligand 1 (PD-L1) ICIs. More specifically, the present invention provides methods that measure kinase activity in samples of said patients.

### BACKGROUND OF THE INVENTION

Tumors evade T cell-mediated destruction via the expression of inhibitory immune checkpoints, including the programmed cell death-1/programmed cell death ligand-1 (PD-1/PD-L1) and cytotoxic T lymphocyte antigen-4 (CTLA-4)/CD80 or CD86 pathways.

The cognate interaction between T cells and antigen presenting cells (APCs), via interaction of the T cell receptor (TCR) with antigen presented in the context of HLA, results in the activation of T cells which subsequently upregulate expression of both CTLA-4 and PD-1. As a result, T cells may display a reduced capacity to become activated, proliferate and exert specific effector functions. Currently, CTLA-4 is thought to play a major role during priming of a T cell response in the lymph node where it directly prevents co-stimulation of T cells via interaction of CD28 with its ligand CD80/86 on APCs. Since CTLA-4 binds with higher affinity to CD80/86, it outcompetes interaction and co-stimulation of T cells via CD28. Moreover, CTLA-4 is constitutively expressed on regulatory T cells (Tregs). Tregs can reduce expression of co-stimulatory CD80/86 molecules on APC by trans-endocytosis thereby preventing T cell activation. PD-1/PD-L1 interaction inhibits T cell activation during the effector phase and is thought to dampen an immune response after antigen eradication in order to prevent immune pathology.

The emergence of immune checkpoint inhibitors (ICIs) is a revolutionary milestone in the field of immunotherapy. ICIs prevent the checkpoint proteins from binding with their partner proteins and thereby reinvigorate antitumor immune responses by interrupting co-inhibitory signalling pathways and stimulate immune-mediated elimination of neoplastic cells. ICIs have been approved for a variety of malignancies and revolutionized the clinical management of multiple malignancies.

The first approved ICI for treating patients with stage 11 or IV melanoma was Ipilimumab, which is an antibody targeting CTLA-4. Ipilimumab inhibits the T-cells and promotes the activation and proliferation of effector T cells. Pembrolizumab and nivolumab are examples of ICIs targeting PD-1. Pembrolizumab and nivolumab are used among others in the treatment of melanoma or non-small cell lung carcinoma (NSCLC) patients, as well as urothelial bladder cancer patients and patients with relapsed or refractory Hodgkin's lymphoma. Atezolizumab and durvalumab are examples of ICIs targeting PD-L1 used among others for the treatment of urothelial carcinoma and NSCLC.

Despite the success of anti-CTLA-4 and anti-PD-1/PD-L1 therapies, durable responses are only obtained in a minority of patients. Furthermore, severe immune-related adverse events (irAEs) are seen in some patients undergoing ICI therapy.

Therefore, the development of predictive biomarkers that can determine the outcome of ICI therapy in a patient before the initiation of a proposed ICI therapy is urgently needed to avoid any adverse effects.

### SUMMARY OF THE INVENTION

The response to an ICI between individuals differs. An ICI can work more or less efficient; but can also lack therapeutic effect, induce adverse drug reactions, toxicity and side effects.

As described in the present invention, the inventors have surprisingly found that the response of a patient in need of an ICI to a PD-1 or PD-L1 ICI can be predicted and/or determined on the basis of the measurement of the kinase activity, preferably protein kinase activity, in a blood sample taken from said patient. The methods according to present invention enable to provide information regarding the efficacy of the targeted pharmacotherapy treatment, and more specifically provide an early determination of the most suited treatment of the patient. Preferably, the measurement of the kinase activity is performed by contacting the blood sample from the patient with one or more kinase substrate(s), preferably protein kinase substrate(s), thereby generating one or more phosphorylation profile(s). Said protein kinase substrate(s) as used herein, is/are preferably peptide(s), protein(s) or peptide mimetic(s). Preferably, the protein kinase substrate(s) each comprise one or more phosphorylation site(s) that can be phosphorylated by the protein kinases present in the sample. Therefore, exposure of the protein kinase substrate(s) to a sample comprising protein kinases results in the phosphorylation of one or more of the phosphorylation site(s) of the protein kinase substrate(s). This phosphorylation activity can be measured using techniques known in the art. Therefore, during the measurement method the kinase enzymes present in the sample will phosphorylate one or more of the phosphorylation site(s) on one or more protein kinase substrate(s).

Present inventors have observed essential differences between kinase activity of a blood sample of patients in need of ICI having a different response to a PD-1 or PD-L1 ICI. More particularly, present inventors have surprisingly found that the aberrant activity of at least two kinases independently selected from within at least two families of kinases selected from the group consisting of: the VEGFR or PDGFR family of kinases; the SRC family of kinases; the SYK family of kinases; the TAM family of kinases; the JakA family of kinases; and the ALK family of kinases, especially selected from within at least two families of kinases selected from the group consisting of the VEGFR or PDGFR family of kinases; the SRC family of kinases; the SYK family of kinases; and the TAM family of kinases; may especially be used for predicting the response of a patient in need of an ICI to a PD-1 or PD-L1 ICI. Surprisingly, such prediction of the response was independent of the origin of the underlying neoplastic disease, such as a tumour, and was observed, inter alia, in patients suffering from melanoma, lung cancer (e.g. NSCLC), bladder cancer and ovarian cancer.

The present invention provides a method for predicting the response of a patient in need of an immune checkpoint inhibitor (ICI), to treatment with a programmed cell death-1 (PD-1) or programmed death-ligand 1 (PD-L1) ICI, comprising the steps of:
(a) measuring the kinase activity of at least two kinases independently selected from within at least two families of kinases selected from the group consisting of: the VEGFR or PDGFR family of kinases; the SRC family of kinases; the SYK family of kinases; the TAM family of kinases; the JakA family of kinases; and the ALK family of kinases, in a blood sample obtained from said patient, thereby providing a kinase activity profile of said blood sample, preferably a blood sample comprising peripheral blood mononuclear cells; and
(b) predicting from said kinase activity profile the response of said patient to treatment with said PD-1 or PD-L1 ICI.

In particular embodiments
the VEGFR or PDGFR family of kinases consists of VEGFR1, VEGFR2, VEGFR3, PDGFRalpha, PDGFRbeta, CSF-1R, Kit, FLT3, and any combination thereof; preferably the VEGFR or PDGFR family of kinases consists of VEGFR1, VEGFR2, VEGFR3, PDGFRalpha, PDGFRbeta, FLT3, and any combination thereof, more preferably the VEGFR or PDGFR family of kinases consists of VEGFR1, VEGFR3, FLT3, and any combination thereof;
the SRC family of kinases consists of SRC, YES, FYN, FGR, LCK, HCK, BLK, LYN, FRK, and any combination thereof; preferably the SRC family of kinases consists of SRC, FYN, BLK, and any combination thereof;
the SYK family of kinases consists of SYK, ZAP-70, and any combination thereof;
the TAM family of kinases consists of TYRO3, AXL, MERTK, and any combination thereof,
the JakA family of kinases consists of JAK1, JAK2, JAK3, and TYK2 and any combination thereof, and/or
the ALK family of kinases consists of ALK, LTK, and any combination thereof.

In particular embodiments, said method comprises measuring the kinase activity of at least two kinases selected from the group consisting of VEGFR1, VEGFR3, FLT3, SRC, FYN, BLK, SYK, ZAP70, TYRO-3, AXL, MER, TRKC, RON, ALK, LTK, and JAK1.

In particular embodiments, said PD-1 or PD-L1 ICI is an antibody.

In particular embodiments, said PD-1 or PD-L1 ICI is selected from the group consisting of Nivolumab, Pembrolizumab, Durvalumab, Atezolizumab, Avelumab, Cemiplimab, Camrelizumab, Sintilimab, Tislelizumab, Toripalimab, and any combination thereof, preferably selected from the group consisting of Nivolumab, Prembrolizumab, and any combination thereof, optionally in combination with a compound selected from the group consisting of Ipilimumab, Tremilimumab, and any combination thereof.

In particular embodiments, said treatment with said PD-1 or PD-L1 ICI is combined with an antineoplastic treatment selected from the group consisting of chemotherapy, radiotherapy, chemoradiotherapy, surgery, an immune checkpoint inhibitor, a kinase inhibitor, a cancer vaccine, an antibody-drug conjugate, a nuclear receptor agonist, a nuclear receptor antagonist, a cytokine modulator, a chemokine modulator, and any combination thereof.

In particular embodiments, said blood sample is a blood sample not being inhibited from clotting by ethylenediaminetetraacetic acid (EDTA).

In particular embodiments, step (b) comprises a step (i) of comparing said kinase activity profile to at least one reference kinase activity profile, wherein said reference kinase activity profile is representative of a good or poor responder to said PD-1 or PD-L1 ICI; and a step (ii) of determining the response of said patient to treatment with said PD-1 or PD-L1 ICI on the basis of the comparison of said kinase activity profile with said at least one reference kinase activity profile.

In particular embodiments, in step (a) said kinase activity is determined by contacting the blood sample with at least two of the protein kinase substrates as listed in Table 2 or 3, thereby providing a phosphorylation profile of said blood sample, said phosphorylation profile comprising the phosphorylation levels of phosphorylation sites present in said at least two protein kinase substrates. In particular embodiments, said patient in need of an ICI is a patient diagnosed with a neoplastic disease, preferably selected from the group consisting of non-small-cell lung carcinoma (NSCLC), bladder cancer, ovarian cancer, prostate cancer, head and neck cancer, colorectal cancer and melanoma.

In particular embodiments, the method as taught herein does not comprise measuring the kinase activity of any kinase other than those selected from the group consisting of: the VEGFR or PDGFR family of kinases; the SRC family of kinases; the SYK family of kinases; the TAM family of kinases; the JakA family of kinases; and the ALK family of kinases.

A further aspect provides the use of the method for predicting the response of a patient in need of an ICI, to treatment with a PD-1 or PD-L1 ICI as taught herein for assessing the response of a patient in need of an ICI to treatment with a PD-1 or PD-L1 ICI.

A further aspect provides the use of the method for predicting the response of a patient in need of an ICI, to treatment with a PD-1 or PD-L1 ICI as taught herein for assessing the pharmaceutical or clinical value of a PD-1 or PD-L1 ICI.

A further aspect provides a kit for predicting the response of a patient in need of an ICI to treatment with a PD-1 or PD-L1 ICI, according to any of claims 1 to 11, comprising means for measuring the kinase activity of at least two kinases independently selected from within at least two families of kinases selected from the group consisting of: the VEGFR or PDGFR family of kinases; the SRC family of kinases; the SYK family of kinases; the TAM family of kinases; the JakA family of kinases; and the ALK family of kinases, in a blood sample obtained from said patient.

In particular embodiments, the means for measuring the kinase activity at least two kinases independently selected from within at least two families of kinases selected from the group consisting of: the VEGFR or PDGFR family of kinases; the SRC family of kinases; the SYK family of kinases; the TAM family of kinases; the JakA family of kinases; and the ALK family of kinases, are at least one array comprising all of the 142 peptide markers as listed in Table 2 or all of the 62 peptide markers as listed in Table 3.

### BRIEF DESCRIPTION OF FIGURES

FIG. 1 Erythrocyte lysis and type of blood collection tubes affect overall kinase activity. The overall kinase activity is depicted as relative kinase activity (%) compared to the appropriate controls which were set at 100%. A) The kinase activity was determined after erythrocyte lysis for 10 or 30 minutes during peripheral blood mononuclear cells (PBMC) sample preparation and is compared to the obtained kinase activity of PBMC prepared without erythrocyte lysis (none). Kinase activity was clearly reduced after erythrocyte lysis during PBMC isolation. B) Blood of healthy donors (n=4) was collected in ethylenediaminetetraacetic acid (EDTA) or sodium heparin anti-coagulated collection tubes and PBMCs were isolated within 4 or 24 hours (h). Reduced kinase activity is demonstrated when the time between blood collection and PBMC isolation increases from isolation within 4 to 24 h. This depends on the type of blood collection tube used, since it is only observed for EDTA collection tube. Kinase activity was suggested to be more stable when sodium heparin collection tubes are used. Error bars indicate the standard deviation (SD). Abbreviations: ethylenediaminetetraacetic acid collection tube (EDTA), sodium heparin collection tube (heparin).

FIG. 2 Schematic overview of the study. Schematic overview of the study. A) Workflow chart showing that kinase activity was measured in baseline PBMC samples using a peptide microarray system consisting of identical arrays, each containing 144 unique protein tyrosine kinase phosphorylation sites. PBMC samples were isolated from blood collected before onset of immune checkpoint inhibitor therapy. The kinase activity profile is analyzed using BioNavigator and Ingenuity Pathway Analysis software. B) The flow chart of the patient selection process. Abbreviations: non-small cell lung carcinoma (NSCLC), anti-programmed cell death 1 (PD-1), immune checkpoint inhibitor (ICI), cytotoxic T-lymphocyte antigen 4 (CTLA-4).

FIG. 3 The extended flow chart of the patient selection process is shown. 174 patients were assessed for eligibility, and 14 technical outliers were removed (8%). The presence of a signal on tyrosine phosphorylation sites was determined based on the detection of a positive trend in the recorded phosphorylation time course. Peptides for which such a trend could not be detected in >75% of the samples were filtered out before further analysis. This resulted in kinase profiles that included 88-113 peptides for analysis, depending on the patient sample cohort. Abbreviations: non-small cell lung carcinoma (NSCLC), anti-programmed cell death 1 (PD-1), immune checkpoint inhibitor (ICI), cytotoxic T-lymphocyte antigen 4 (CTLA-4).

FIG. 4 Baseline kinase activity profiles and classification analysis of patients treated with CTLA-4 blockade. Heat maps showing kinase activity profiles of separate cohorts of melanoma patients who were treated with cytotoxic T-lymphocyte associated antigen 4 (CTLA-4) immune checkpoint inhibitors (ICIs): A) discovery cohort Mel-CTLA4-A and B) confirmation cohort Mel-CTLA4-B. The heat map displays binary grouping into patients who benefit from treatment (responders) and who do not benefit from treatment (non-responders). The columns represent subjects sorted from left to right according to treatment response; the rows represent peptides sorted according to Pearsons correlation coefficient with treatment response, such that the peptides with a relatively higher phosphorylation signal in the non-responders are shown at the top of the map and the peptides with a relatively high signal in the responders are shown near the bottom. The values are scaled per row to zero mean and unit variance for the purpose of this visualization. Furthermore, the classification analysis of these patient sample cohorts are shown: C) discovery cohort Mel-CTLA4-A and D) confirmation cohort Mel-CTLA4-B. The bar graphs show for each patient the prediction index obtained by cross validation of a partial least squares discriminant analysis (PLS-DA) model (see methods section in Example 1). If the prediction index >0 the patient is predicted to be a responder, and a non-responder otherwise. The black or white filling of the bars indicates the actual clinical response class of the patients.

FIG. 5 Baseline kinase activity profiles and classification analysis of patients treated with PD-1 blockade. Heat maps showing the kinase activity profile of separate cohorts of melanoma patients who were treated with programmed cell death protein 1 (PD-1) ICIs: A) discovery cohort Mel-PD1-A, B) confirmation cohort Mel-PD1-B and C) confirmation cohort non-small cell lung carcinoma (NSCLC)-PD1. The heat map displays binary grouping into patients who benefit from treatment (responders) and who do not benefit from treatment (non-responders). The columns represent subjects sorted from left to right according to treatment response; the rows represent peptides sorted according to correlation coefficient with treatment response, such that the peptides with a relatively higher phosphorylation signal in the non-responders are shown at the top of the map and the peptides with a relatively high signal in the responders are shown near the bottom. The values are scaled per row to zero mean and unit variance for the purpose of this visualization. Furthermore, the classification analysis of these cohorts are shown: D) discovery cohort Mel-PD1-A, E) confirmation cohort Mel-PD1-B and F) confirmation cohort NSCLC-PD1. The bar graphs show for each patient the prediction index obtained by cross validation of a PLS-DA model. If the prediction index>0 the patient is predicted to be a responder, and a non-responder otherwise. The black or white filling of the bars indicates the actual clinical response class of the patients.

FIG. 6 Phylogenetic tree analysis. Results of upstream kinase analysis for the anti-PD1 treated cohorts, Mel-PD1-A (A), Mel-PD1-B (B), and NSCLC-PD1 (C). The Coral tool was applied to annotate the results on a phylogenetic tree for protein tyrosine kinases (TK) (Cell Signaling Technologies Inc.). A) shows the individual kinases (grey discs) with a predicted higher activity in responders for the Mel-PD1-A cohort. B) upper panel shows the individual kinases (grey discs) with a predicted higher activity in the responders and lower panel shows the individual kinases (grey discs) with a predicted higher activity in the non-responders for the Mel-PD1-B cohort. C) upper panel shows the individual kinases (grey discs) with a predicted higher activity in the responders and lower panel shows the individual kinases (grey discs) with a predicted higher activity in the non-responders for the NSCLC-PD1 cohort. The diameter of the circle indicates the specificity score of the corresponding kinase (a higher score indicates that according to the analysis a kinase is more likely to contribute to the observed phosphorylation changes). Protein descriptions for the kinases listed in the tree of FIG. 6 can be consulted via the protein database of U.S. government's National Center for Biotechnology Information (NCBI) (http://www.ncbi.nlm.nih.gov/) or via the homepage of UniProt (https://www.uniprot.org/).

FIG. 7 Pathway analysis. Top canonical pathways are schematically shown based on the baseline kinase activity profile of cohort A) Mel-PD1-A and B) NSCLC-PD1. “1” (positive z-score) indicates an expected upregulation of the pathway in patients who benefit from anti-PD1 treatment (responders), “2” (negative z-score) indicates an expected downregulation of the pathway in responders. “3” represents canonical pathways without an expected activity pattern. The significance values indicate the probability of the association of the involved kinases with the canonical pathway by random chance alone, cutoff was set at a B-H p-value >12. Ranking was based on the trend and z-score.

FIG. 8 Kaplan Meier survival plots of the progression-free survival (PFS) of the cohort of melanoma patients, stratified according to predicted response.

FIG. 9-A) Phylogenetic tree of tyrosine kinases on which individual kinases with a predicted higher activity in melanoma patients with PD (Non-responders), are annotated with a grey disc (a larger diameter indicates a higher significance). B), as A) but for kinases with a higher activity in melanoma patients with no-PD (responders). Protein descriptions for the kinases listed in the tree of FIG. 9 can be consulted via the protein database of U.S. government's National Center for Biotechnology Information (NCBI) (http://www.ncbi.nlm.nih.gov/) or via the homepage of UniProt (https://www.uniprot.org/).

FIG. 10 Kaplan Meier survival plots of the PFS of the cohort of NSCLC patients, stratified according to predicted response.

FIG. 11 A) Phylogenetic tree of tyrosine kinases on which individual kinases with a predicted higher activity in NSCLC patients with PD (Non-responders), are annotated with a grey disc (a larger diameter indicates a higher significance). B) as A) but for kinases with a higher activity in NSCLC patients with no-PD (responders). Protein descriptions for the kinases listed in the tree of FIG. 11 can be consulted via the protein database of U.S. government's National Center for Biotechnology Information (NCBI) (http://www.ncbi.nlm.nih.gov/) or via the homepage of UniProt (https://www.uniprot.org/).

FIG. 12 Kaplan Meier survival plots of the PFS of the combined cohort, stratified according to predicted response.

FIG. 13 A) Phylogenetic tree of tyrosine kinases on which individual kinases with a predicted higher activity in melanoma and NSCLC patients with PD (Non-responders), are annotated with a grey disc (a larger diameter indicates a higher significance). B) as A) but for kinases with a higher activity in melanoma and NSCLC patients with no-PD (responders). Protein descriptions for the kinases listed in the tree of FIG. 13 can be consulted via the protein database of U.S. government's National Center for Biotechnology Information (NCBI) (http://www.ncbi.nlm.nih.gov/) or via the homepage of UniProt (https://www.uniprot.org/).

FIG. 14 A) Phylogenetic tree of tyrosine kinases on which individual kinases with a predicted higher activity in ovarian cancer patients with PD (Non-responders), are annotated with a grey disc (a larger diameter indicates a higher significance). B) as A) but for kinases with a higher activity in ovarian cancer patients with no-PD (Responders). Protein descriptions for the kinases listed in the tree of FIG. 14 can be consulted via the protein database of U.S. government's National Center for Biotechnology Information (NCBI) (http://www.ncbi.nlm.nih.gov/) or via the homepage of UniProt (https://www.uniprot.org/).

FIG. 15 Table 2: list of 142 peptide markers comprising phosphorylation sites used for determining the kinase activity of the at least two families of kinases selected from the group consisting of the VEGFR or PDGFR family of kinases; the SRC family of kinases; the SYK family of kinases; and the TAM family of kinases; the JakA family of kinases; the ALK family of kinases, and optionally the family of kinases consisting of TRKC, RON, and any combination thereof, their sequence and SEQ ID NO. The name of the peptide markers refers to the associated proteins and to the start and the end position of the amino acid sequence.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of ±10% or less, preferably ±5% or less, more preferably ±1% or less, and still more preferably ±0.1% or less of and from the specified value, insofar such variations are appropriate to perform in the disclosed invention. It is to be understood that the value to which the modifier “about” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may be referring to the same embodiment. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

Efforts from others to predict the clinical response to immune checkpoint inhibitor (ICI) therapy resulted in various biomarkers, including PD-L1 expression in tumor tissue and tumor mutational burden (TMB). The predictive performance of these biomarkers however is not sufficient and is complicated by both the availability of tissue and the inter- and intra-tumoral heterogeneity. Present inventors demonstrated that the kinase activity of at least two kinases from within at least two families of kinases selected from the group consisting of the VEGFR or PDGFR family of kinases, the SRC family of kinases, the SYK family of kinases, the TAM family of kinases, the JakA family of kinases and the ALK family of kinases, preferably selected from the group consisting of the VEGFR or PDGFR family of kinases, the SRC family of kinases, the SYK family of kinases, and the TAM family of kinases, more preferably the VEGFR or PDGFR family of kinases and the SRC family of kinases, in a blood sample of a subject, reflects the biological mechanisms of response to PD-1 or PD-L1 ICIs and allows predicting the response of the subject to treatment with a PD-1 or PD-L1 ICI. As use is made of a blood sample, present invention allows predicting the response of a subject to treatment with a PD-1 or PD-L1 ICI, while being minimally invasive and leading to less discomfort to the patient compared to prediction methods known in the art. Furthermore, the present method allows predicting the response to treatment with a PD-1 or PD-L1 ICI of any subject in need of treatment of an ICI and should not be considered to be limited to a very specific group of patients diagnosed with a very specific type of neoplastic disease. Present inventors found that the present method works, for instance, particularly well for predicting the response to treatment with a PD-1 or PD-L1 ICI of patients diagnosed with NSCLC, melanoma, ovarian cancer, and/or bladder cancer.

A first aspect provides a method for predicting the response of a patient in need of an immune checkpoint inhibitor (ICI), to treatment with a PD-1 or PD-L1 ICI, comprising the steps of:

(a) measuring the kinase activity of
at least 2, at least 3, at least 4, at least 5, at least 6, at least 7, at least 8, at least 9, at least 10, at least 11, at least 12, at least 13, or at least 14, kinases independently selected from within at least 2, at least 3, at least 4, at least 5, or at least 6, families of kinases selected from the group consisting of: the VEGFR or PDGFR family of kinases; the SRC family of kinases; the SYK family of kinases; the TAM family of kinases; the JakA family of kinases; and the ALK family of kinases, preferably of at least 2, at least 3, at least 4, at least 5, at least 6, at least 7, at least 8, at least 9, at least 10, or at least 11, kinases independently selected from within at least 2, at least 3, or at least 4 families of kinases selected from the group consisting of: the VEGFR or PDGFR family of kinases; the SRC family of kinases; the SYK family of kinases; and the TAM family of kinases, more preferably at least 2, at least 3, at least 4, at least 5, or at least 6, kinases independently selected from within the VEGFR or PDGFR family of kinases and the SRC family of kinases;
in a blood sample obtained from said patient, thereby providing a kinase activity profile of said blood sample; and
(b) predicting from said kinase activity profile the response of said patient to treatment with said PD-1 or PD-L1 ICI.

In other words, provided herein is a method for determining sensitivity which may also be denoted as responsiveness or susceptibility) or resistance (which may also be denoted as unresponsiveness or insusceptibility) of a patient in need of an ICI to treatment with a PD-1 or PD-L1 ICI, comprising the steps of:
(a) measuring the kinase activity of
   at least 2, at least 3, at least 4, at least 5, at least 6, at least 7, at least 8, at least 9, at least 10, at least 11, at least 12, at least 13, or at least 14, kinases independently selected from within at least 2, at least 3, at least 4, at least 5, or at least 6 families of kinases selected from the group consisting of: the VEGFR or PDGFR family of kinases; the SRC family of kinases; the SYK family of kinases; the TAM family of kinases; the JakA family of kinases; and the ALK family of kinases, preferably of at least 2, at least 3, at least 4, at least 5, at least 6, at least 7, at least 8, at least 9, at least 10, or at least 11, kinases independently selected from within at least 2, at least 3, or at least 4, families of kinases selected from the group consisting of: the VEGFR or PDGFR family of kinases; the SRC family of kinases; the SYK family of kinases; and the TAM family of kinases, more preferably at least 2, at least 3, at least 4, at least 5, or at least 6, kinases independently selected from within the VEGFR or PDGFR family of kinases and the SRC family of kinases;
   in a blood sample obtained from said patient, thereby providing a kinase activity profile of said blood sample; and
(b) determining from said kinase activity profile the sensitivity of said patient to treatment with said PD-1 or PD-L1 ICI.

In certain embodiments, the methods or uses as taught herein are useful for predicting an outcome of treatment with a PD-1 or PD-L1 ICI in a patient in need of an ICI.

In certain embodiments, the kinase activity of at least two kinases independently selected from within at least two families of kinases selected from the group consisting of the VEGFR or PDGFR family of kinases; the SRC family of kinases; the SYK family of kinases; the TAM family of kinases, the JakA family of kinases, and the ALK family of kinases; in a blood sample obtained from said patient, may be employed as a marker for the patient's sensitivity to treatment with a PD-1 or PD-L1 ICI. The terms “marker” and “biomarker” are widespread in the art and commonly broadly denote a biological component or a biological molecule, more particularly an endogenous biological component or molecule, or a detectable portion thereof, whose qualitative and/or quantitative evaluation in a tested subject, such as by means of evaluating a biological sample from the subject, is predictive or informative with respect to one or more aspects of the tested subjects' phenotype and/or genotype. The phrases “predicting the response” and “determining the response” may be used interchangeably herein.

The terms “predicting”, “prediction” or “predictive” as used herein refers to an advance declaration, indication or foretelling of a response or reaction to a therapy in a subject, preferably wherein said subject has not (yet) been treated with the therapy. For example, a prediction of sensitivity (or responsiveness or susceptibility) to treatment with a PD-1 or PD-L1 ICI in a subject may indicate that the subject will respond or react to the treatment, for example within a certain time period, e.g., so that the subject will have a clinical benefit (e.g., will display reduced tumour load, or will display complete or partial response, will display stable disease for a period of at least 90 days, will display late (after 140 days) or no disease progression) from the treatment. A prediction of insensitivity (or unresponsiveness or insusceptibility or resistance) to treatment with an a PD-1 or PD-L1 ICI in a subject may indicate that the subject will minimally or not respond or react to the treatment, for example within a certain time period, e.g., so that the subject will have no clinical benefit (e.g., will not display a therapeutically meaningful reduction in tumour load, will display disease progression, will display early (within 140 days) disease progression) from the treatment.

In certain embodiments, the response of said patient to said PD-1 or PD-L1 ICI is the time to progression of disease upon treatment with said PD-1 or PD-L1 ICI, wherein a good responder has a late (e.g. more than 140 days after treatment with said PD-1 or PD-L1 ICI) or no progression of disease upon treatment with said PD-1 or PD-L1 ICI and the poor responder has an early progression (e.g. less than 140 days after treatment with said PD-1 or PD-L1 ICI) of disease upon treatment with said PD-1 or PD-L1 ICI.

The terms “sensitivity”, “responsiveness” or “susceptibility” may be used interchangeably herein and refer to the quality that predisposes a subject having a neoplastic disease to be sensitive or reactive to treatment with a PD-1 or PD-L1 ICI. A subject is “sensitive”, “responsive” or “susceptible” (which terms may be used interchangeably) to treatment with a PD-1 or PD-L1 ICI if the subject will have a clinical benefit from the treatment. A neoplastic tissue, such as a tumour, is “sensitive”, “responsive”, or “susceptible” to treatment with an antineoplastic agent if the proliferation rate of the neoplastic tissue is inhibited as a result of contact with a therapeutically effective amount of the PD-1 or PD-L1 ICI, compared to the proliferation rate of the neoplastic tissue in the absence of contact with the PD-1 or PD-L1 ICI.

The terms “insensitivity”, “unresponsiveness”, “insusceptibility” or “resistance” may be used interchangeably herein and refer to the quality that predisposes a subject in need of an immune checkpoint inhibitor to a minimal (e.g. clinically insignificant) or no response to treatment with a PD-1 or PD-L1 ICI. A subject is “insensitive”, “unresponsive”, “unsusceptible” or “resistant” (which terms may be used interchangeably) to treatment with a PD-1 or PD-L1 ICI if the subject will have no clinical benefit from the treatment. A neoplastic tissue, including a tumour, is “insensitive”, “unresponsive”, “unsusceptible” or “resistant” to treatment with a PD-1 or PD-L1 ICI if the proliferation rate of the neoplastic tissue is not inhibited, or inhibited to a very low (e.g. therapeutically insignificant) degree, as a result of contact with a therapeutically effective amount of the PD-1 or PD-L1 ICI, compared to the proliferation rate of the neoplastic tissue in the absence of contact with the PD-1 or PD-L1 ICI.

The methods as disclosed herein may allow making a prediction that a patient in need of an ICI will be responsive to treatment with a PD-1 or PD-L1 ICI or will be non-responsive to treatment with a PD-1 or PD-L1 ICI. This may in certain embodiments include predicting that a patient in need of an ICI will have a comparatively low probability (e.g., less than 50%, less than 40%, less than 30%, less than 20% or less than 10%) of being responsive (or being a responder) to treatment with a PD-1 or PD-L1 ICI; or that a patient in need of an ICI will have a comparatively high probability (e.g., at least 50%, at least 60%, at least 70%, at least 80% or at least 90%) of being responsive (or of being a responder) to treatment with a PD-1 or PD-L1 ICI.

The present methods of evaluating kinase activity to provide information as to the patient's responsiveness to a PD-1 or PD-L1 ICI are generally performed in vitro, on a blood sample obtained from a patient. The term “in vitro” generally denotes outside, or external to, animal or human body. The term “ex vivo” typically refers to tissues or cells removed from an animal or human body and maintained or propagated outside the body, e.g., in a culture vessel. The term “in vitro” as used herein should be understood to include “ex vivo”. The term “in vivo” generally denotes inside, on, or internal to, animal or human body.

For purposes of the present invention, and as used herein the term “kinase activity” or “protein kinase activity” refer to the formation of reaction product(s) by a certain amount of kinase or protein kinase acting on a substrate during the course of the assay.

Protein kinase activity is referred to as the activity of protein kinases. A protein kinase is a generic name for all enzymes that transfer a phosphate to a protein. About two percent of the human genome contains transcription information for the formation of protein kinases. Currently, there are about 518 known different protein kinases. However, because three to four percent of the human genome is a code for the formation of protein kinases, there may be many more separate kinases in the human body.

A protein kinase is a kinase enzyme that modifies other proteins by covalently coupling phosphate groups to them. This process or activity is also referred to as phosphorylation. Phosphorylation can therefore be regarded as the process of the addition of a phosphate group to a substrate. Phosphorylation usually results in a functional change of the substrate by changing kinase activity, cellular location, or association with other proteins. Up to 30 percent of all proteins may be modified by kinase activity, and kinases are known to regulate the majority of cellular pathways, especially those involved in signal transduction, the transmission of signals within the cell. The chemical activity of a kinase involves removing a phosphate group from ATP or GTP and covalently attaching it to amino acids such as serine, threonine, tyrosine, histidine, aspartic acid and/or glutamic acid that have a free hydroxyl group. Most known kinases act on both serine and threonine, others act on tyrosine, and a number act on all serine, threonine and tyrosine.

The protein kinase activity monitored with the method of the present invention is preferably directed to protein kinases acting towards tyrosine and/or serine, or towards threonine, preferably acting on both serine and threonine, on tyrosine or on serine, threonine and tyrosine and more preferably the method of the present invention if preferably directed to protein kinases acting towards tyrosine. Protein kinases are distinguished by their ability to phosphorylate substrates on discrete sequences. These sequences have been determined by sequencing the amino acids around the phosphorylation sites.

Because protein kinases have profound effects on a cell, their activity is highly regulated. Kinases are turned on or off by for instance phosphorylation, by binding of activator proteins or inhibitor proteins, or small molecules, or by controlling their location in the cell relative to their substrates. Deregulated activity is a frequent cause of disease, particularly cancer, where kinases regulate many aspects that control cell growth, movement and death. Kinases also play an important role in the activation of cells of the immune system (for example see Weiss A., Kinases and phosphatases of the immune system, Immunological Reviews 2009, Vol. 228: 5-8). Therefore, monitoring the protein kinase activity in tissues can be of great importance and a large amount of information can be obtained when comparing the kinase activity of different tissue samples.

For example, as described herein, at least two kinases independently selected from within at least two families of kinases selected from the group consisting of: the VEGFR or PDGFR family of kinases; the SRC family of kinases; the SYK family of kinases; the TAM family of kinases; the JakA family of kinases; and the ALK family of kinases, present in a blood sample from patients in need of an ICI will phosphorylate protein kinase substrates differently depending on the response to the PD-1 or PD-L1 ICI with which the patient is envisaged to be treated or is being treated. Accordingly, phosphorylation signals differ between the blood samples, resulting in phosphorylation patterns that differ depending on response to the PD-1 or PD-1 ICI.

For purposes of the present invention, and as used herein the term “pharmacotherapy”, or “pharmacotherapeutics” or “drug treatment” refers to the use of a pharmaceutical drug, also referred to as medicine or medicament wherein said pharmacotherapy is intended for use in the diagnosis, cure, mitigation, treatment, or prevention of disease.

The kinase activity of at least two kinases independently selected from within at least two families of kinases selected from the group consisting of: the VEGFR or PDGFR family of kinases; the SRC family of kinases; the SYK family of kinases; the TAM family of kinases; the JakA family of kinases; and the ALK family of kinases, in a blood sample obtained from a patient in need of an ICI could serve as an accurate early indicator for therapeutic response in a patient to measure the effectiveness of candidate PD-1 or PD-L1 ICIs.

The term “VEGFR family of kinases” as used herein refers to a family of transmembrane proteins, namely receptor tyrosine kinases, which transduce signals from the extracellular environment to the cytoplasm and nucleus.

The term “PDGFR family of kinases” as used herein refers to a family of transmembrane proteins, namely receptor tyrosine kinases, which transduce signals from the extracellular environment to the cytoplasm and nucleus.

The term “SRC family of kinases” as used herein refers to a family protein, namely non-receptor tyrosine kinases, which transduce signals in the cytoplasm.

The term “SYK family of kinases” as used herein refers to a family of proteins, namely non-receptor tyrosine kinases, which transduce signals in the cytoplasm.

The term “TAM family of kinases” as used herein refers to a family of transmembrane proteins, namely receptor tyrosine kinases, which transduce signals from the extracellular environment to the cytoplasm and nucleus. The TAM family of RTKs is distinguished from other RTK families by a conserved amino acid sequence, KW (I/L)A(I/L)ES (SEQ ID NO: 143), within the kinase domain (cytosolic region). Also the adhesion molecule-like domains in the extracellular region have conserved sequences.

The term “JakA family of kinases”, “JAKA family of kinases”, “JAK family of kinases” or “janus kinase family of kinases” as used herein refers to a family of non-receptor tyrosine kinases that transduce signals via the JAK-signal transducer and activator of transcription (STAT) pathway.

The term “ALK family of kinases” as used herein refers to a family of receptor tyrosine kinases that transduce signals via multiple downstream signal transduction pathways, including but not limited to the mitogen-activated protein kinase (MAPK)-extracellular-signal-regulated kinase (ERK) and the JAK-STAT pathway.

Exemplary human (Homo sapiens) members of kinase of the VEGFR or PDGFR family of kinases include:
Fms-like tyrosine kinase 1 (FLT1), also known as Vascular endothelial growth factor receptor 1 (VEGFR1), with UniprotID P17948 (i.e. VEGFR family),
Fms-like tyrosine kinase 3 (FLT3) with UniprotID P36888 (i.e. PDGFR family),
Fms-like tyrosine kinase 4 (FLT4), also known as Vascular endothelial growth factor receptor 3 (VEGFR3), with UniprotID P35916 (i.e. VEGFR family),
macrophage colony-stimulating factor 1 receptor (CSF-1R) with UniprotID P07333 (i.e. PDGFR family),
Mast/stem cell growth factor receptor Kit (Kit) with UniprotID P10721 (i.e. PDGFR family),
Platelet-derived growth factor receptor alpha (PDGFRalpha) with UniprotID P16234 (i.e. PDGFR family),
Platelet-derived growth factor receptor beta (PDGFRbeta) with UniprotID P09619 (i.e. PDGFR family),
Kinase insert domain receptor (KDR), also known as Vascular endothelial growth factor receptor 2 (VEGFR2), with UniprotID P35968 (i.e. VEGFR family).

Furthermore, exemplary human (Homo sapiens) members of kinase of the SRC family of kinases include:
proto-oncogene tyrosine-protein kinase Src (SRC), with UniprotID P12931,
B lymphocyte kinase (BLK) with UniprotID P51451,
Leukocyte C-terminal Src kinase (LCK) with UniprotID P06239,
Proto-oncogene c-Fyn (Fyn) with UniprotID P06241,
Tyrosine-protein kinase Yes (YES) with UniprotID P07947,
Breast tumor kinase (BRK) with UniprotID Q13882,
FGR with UniprotID P09769,
Hematopoietic cell kinase (HCK) with UniprotID P08631,
LYN with UniprotID P07948,
FYN-related kinase (FRK) with UniprotID P42685, and
SRMS with UniprotID Q9H3Y6.

Furthermore, exemplary human (Homo sapiens) members of kinase of the SYK family of kinases include:
Spleen tyrosine kinase (Syk) with UniprotID P43405, and
70 kDa zeta-chain associated protein (ZAP70) with UniprotID P43403.

Exemplary human (Homo sapiens) members of the TAM family of RTKs include
AXL receptor tyrosine kinase with NCBI Genbank Gene ID: 558, Swissprot entry P30530, Genbank RefSeq for one representative amino acid sequence followed by the Genbank sequence version NP_001265528.1 and Genbank RefSeq for one representative mRNA sequence followed by the Genbank sequence version NM_001278599.1;
MER proto-oncogene, tyrosine kinase with NCBI Genbank Gene ID: 10461, Swissprot entry Q12866, Genbank RefSeq for one representative amino acid sequence followed by the Genbank sequence version NP_006334.2 and Genbank RefSeq for one representative mRNA sequence followed by the Genbank sequence version NM_006343.2; and

TYRO3 protein tyrosine kinase with NCBI Genbank Gene ID: 7301, Swissprot entry: Q06418, Genbank RefSeq for one representative amino acid sequence followed by the Genbank sequence version NP_001317193.1 and Genbank RefSeq for one representative mRNA sequence followed by the Genbank sequence version NM_001330264.1. Exemplary human (Homo sapiens) members of the JakA family of kinases include:
JAK1 with UniprotID P23458;
JAK2 with UniprotID 060674;
JAK3 with UniprotID P52333; and
TYK2 with UniprotID P29597.

Examplary (Homo sapiens) members of the ALK family of kinases include:
ALK with UniprotID Q9UM73; and
LTK with UniprotID P29376.

In particular embodiments, the VEGFR or PDGFR family of kinases consists of (or consists of a group of kinases selected from the group consisting) VEGFR1, VEGFR2, VEGFR3, PDGFRalpha, PDGFRbeta, CSF-1R, Kit, FLT3, and any combination thereof; preferably VEGFR1, VEGFR2, VEGFR3, PDGFRalpha, PDGFRbeta, FLT3, and any combination thereof, more preferably the VEGFR or PDGFR family of kinases consists of VEGFR1, VEGFR3, FLT3, and any combination thereof.

In particular embodiments, the SRC family of kinases consists of (or consists of a group of kinases selected from the group consisting) SRC, YES, FYN, FGR, LCK, HCK, BLK, LYN, FRK, and any combination thereof; preferably the SRC family of kinases consists of SRC, FYN, BLK and any combination thereof. In particular embodiments, the SYK family of kinases consists of (or consists of a group of kinases selected from the group consisting) SYK, ZAP-70, and any combination thereof.

In particular embodiments, the TAM family of kinases consists of (or consists of a group of kinases selected from the group consisting) TYRO3, AXL, MERTK, and any combination thereof.

In particular embodiments, the JakA family of kinases consists of JAK1, JAK2, JAK3, and TYK (preferably TYK2) and any combination thereof, and/or

In particular embodiments, the ALK family of kinases consists of ALK, LTK, and any combination thereof. In particular embodiments, if the method as taught herein comprises measuring the kinase activity of at least two kinases independently selected from within at least two families of kinases selected from the group consisting of: the VEGFR or PDGFR family of kinases; the SRC family of kinases; the SYK family of kinases; the TAM family of kinases; the JakA family of kinases; and the ALK family of kinases, the method may further comprise measuring the kinase activity of at least one kinase selected from the group of kinases consisting of tropomyosin receptor kinase C (TRKC), RON, and any combination thereof.

In particular embodiments, if the method as taught herein comprises measuring the kinase activity of at least two kinases independently selected from within at least two families of kinases selected from the group consisting of: the VEGFR or PDGFR family of kinases; the SRC family of kinases; the SYK family of kinases; and the TAM family of kinases; the method may further comprise measuring the kinase activity of at least one kinase selected from the group of kinases consisting of tropomyosin receptor kinase C (TRKC), RON, ALK and any combination thereof. For example, TRKC, also known as NT-3 growth factor receptor (NTRK3), may be human TRKC with UniprotID Q16288.

For example, RON, also known as macrophage-stimulating protein receptor (MSTR1), may be human RON with UniprotID Q04912.

In particular embodiments, the method for predicting the response of a patient as taught herein comprises measuring the kinase activity of:

at least 2, at least 3, at least 4, at least 5, at least 6, at least 7, at least 8, at least 9, at least 10, or at least 11, at least 12, at least 13, at least 14, at least 15, at least 16, at least 17, at least 18, at least 19, at least 20, at least 21, at least 22, at least 23, at least 24, at least 25, at least 26, at least 27 or at least 28, preferably at least 14, kinases independently selected from within at least 2, at least 3, at least 4, at least 5 or at least 6, preferably at least 4, families of kinases selected from the group consisting of:
the VEGFR or PDGFR family of kinases consisting of VEGFR1, VEGFR2, VEGFR3, PDGFRalpha, PDGFRbeta, CSF-1R, Kit, FLT3, and any combination thereof;
the SRC family of kinases consisting of SRC, YES, FYN, FGR, LCK, HCK, BLK, LYN, FRK, and any combination thereof;
the SYK family of kinases consisting of SYK, ZAP-70, and any combination thereof;
the TAM family of kinases consisting of TYRO3, AXL, MERTK, and any combination thereof;
the JakA family of kinases consisting of JAK1, JAK2, JAK3, and TYK2 and any combination thereof; and
the ALK family of kinases consisting of ALK, LTK, and any combination thereof, and optionally at least one, preferably two, kinases of the group consisting of TRKC, RON, and any combination thereof.

In particular embodiments, the method for predicting the response of a patient as taught herein comprises measuring the kinase activity of:

at least 2, at least 3, at least 4, at least 5, at least 6, at least 7, at least 8, at least 9, at least 10, or at least 11, at least 12, at least 13, or at least 14, preferably at least 14, kinases independently selected from within at least 2, at least 3, at least 4, at least 5, or at least 6, preferably at least 4, families of kinases selected from the group consisting of:
the VEGFR or PDGFR family of kinases consisting of VEGFR1, VEGFR3, FLT3, and any combination thereof;
the SRC family of kinases consisting of SRC, FYN, BLK and any combination thereof;
the SYK family of kinases consisting of SYK, ZAP-70, and any combination thereof;
the TAM family of kinases consisting of TYRO3, AXL, MERTK, and any combination thereof;
the JakA family of kinases consists of JAK1, and any combination thereof; and
the ALK family of kinases consists of ALK, LTK, and any combination thereof, and optionally at least one kinase of the group consisting of TRKC, RON, and any combination thereof.

In particular embodiments, the method for predicting the response of a patient as taught herein comprises measuring the kinase activity of:

at least 2, at least 3, at least 4, at least 5, at least 6, at least 7, at least 8, at least 9, at least 10, or at least 11, preferably at least 11, kinases independently selected from within at least 2, at least 3, or at least 4, preferably at least 4, families of kinases selected from the group consisting of:
the VEGFR or PDGFR family of kinases consisting of VEGFR1, VEGFR3, FLT3, and any combination thereof;
the SRC family of kinases consisting of SRC, FYN, BLK and any combination thereof;
the SYK family of kinases consisting of SYK, ZAP-70, and any combination thereof; and
the TAM family of kinases consisting of TYRO3, AXL, MERTK, and any combination thereof;
   and optionally at least one, at least two, or at least three, preferably at least three, kinases of the group consisting of TRKC, RON, ALK and any combination thereof.

In particular embodiments, the method for predicting the response of a patient as taught herein comprises measuring the kinase activity of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or all 30 kinases selected from the group consisting of VEGFR1, VEGFR2, VEGFR3, PDGFRalpha, PDGFRbeta, CSF-1R, Kit, FLT3, SRC, YES, FYN, FGR, LCK, HCK, BLK, LYN, FRK, SYK, ZAP-70, TYRO3, AXL, MERTK, JAK1, JAK2, JAK3, TYK2, ALK, LTK, TRKC, and RON.

In particular embodiments, the method for predicting the response of a patient as taught herein comprises measuring the kinase activity of at least 2, at least 3, at least 4, at least 5, at least 6, at least 7, at least 8, at least 9, at least 10, at least 11, at least 12, at least 13, at least 14, at least 15, at least 16, at least 17, at least 18 or all 19 kinases selected from the group consisting of VEGFR1, VEGFR3, FLT3, SRC, FYN, BLK, SYK, ZAP70, TYRO-3, AXL, MER, TRKC, RON, ALK, LTK, JAK1, JAK2, JAK3, and TYK2.

In particular embodiments, the method for predicting the response of a patient as taught herein comprises measuring the kinase activity of at least 2, at least 3, at least 4, at least 5, at least 6, at least 7, at least 8, at least 9, at least 10, at least 11, at least 12, at least 13, at least 14, at least 15, or all 16 kinases selected from the group consisting of VEGFR1, VEGFR3, FLT3, SRC, FYN, BLK, SYK, ZAP70, TYRO-3, AXL, MER, TRKC, RON, ALK, LTK and JAK1 as listed in Table 1.

In particular embodiments, the method for predicting the response of a patient as taught herein comprises measuring the kinase activity of at least 2, at least 3, at least 4, at least 5, at least 6, at least 7, at least 8, at least 9, at least 10, at least 11, at least 12, at least 13, or all 14 kinases selected from the group consisting of VEGFR1, VEGFR3, FLT3, SRC, FYN, BLK, SYK, ZAP70, TYRO-3, AXL, MER, TRKC, RON and ALK.

TABLE 1
list of 16 kinases used for predicting the response of
a patient in need of an ICI to a PD-1 or PD-L1 ICI.
Kinase

VEGFR1
VEGFR3
FLT3
SRC
FYN
BLK
SYK
ZAP70
TYRO-3
AXL
MER
TRKC
RON
ALK
LTK
JAK1

The skilled person will understand that when the number of kinases of which the activity is determined according to the method as taught herein increases, so will increase the specificity, accuracy and sensitivity of the method according to the present invention. The highest method accuracy will be obtained when the kinase activity of all kinases selected from the group consisting of VEGFR1, VEGFR3, FLT3, SRC, FYN, BLK, SYK, ZAP70, TYRO-3, AXL, MER, TRKC, RON, and ALK, all kinases selected from the group consisting of VEGFR1, VEGFR3, FLT3, SRC, FYN, BLK, SYK, ZAP70, TYRO-3, AXL, MER, TRKC, RON, ALK, LTK, and JAK1, or all kinases selected from the group consisting of VEGFR1, VEGFR3, FLT3, SRC, FYN, BLK, SYK, ZAP70, TYRO-3, AXL, MER, TRKC, RON, ALK, LTK, JAK1, JAK2, JAK3, and TYK2 is used.

Accordingly, in particular embodiments, the method for predicting the response of a patient as taught herein comprises measuring the kinase activity of all of VEGFR1, VEGFR3, FLT3, SRC, FYN, BLK, SYK, ZAP70, TYRO-3, AXL, MER, TRKC, RON, ALK, LTK, and JAK1.

The person skilled in the art will understand that the determination of the kinase activity of at least two kinases independently selected from within at least two families of kinases selected from the group consisting of:
the VEGFR or PDGFR family of kinases, preferably the VEGFR or PDGFR family of kinases consisting of VEGFR1, VEGFR2, VEGFR3, PDGFRalpha, PDGFRbeta, CSF-1R, Kit, FLT3, and any combination thereof;
the SRC family of kinases, preferably the SRC family of kinases consisting of SRC, YES, FYN, FGR, LCK, HCK, BLK, LYN, FRK, and any combination thereof;
the SYK family of kinases, preferably the SYK family of kinases consisting of SYK, ZAP-70, and any combination thereof;
the TAM family of kinases, preferably the TAM family of kinases consists of TYRO3, AXL, MERTK, and any combination thereof;
the JakA family of kinases, preferably the JakA family of kinases consisting of JAK1, JAK2, JAK3, TYK2, and any combination thereof; and
the ALK family of kinases, preferably the ALK family of kinases consisting of ALK, LTK, and any combination thereof,
   and optionally at least one kinases of the group consisting of TRKC, RON, and any combination thereof, provides a kinase activity profile or a kinase activity signature of said sample.

As used in the present invention, the term “kinase activity profile” or “kinase activity signature” refers to a data set representative for the kinase activity (presence, absence and/or quantity, enzymatic activity, preferably enzymatic activity) of, preferably one or more, kinases present in the sample. A kinase activity profile can also be generated when determining the activity of the kinases as taught herein in different test conditions such as for example by comparing the kinase activity of a sample in the presence and absence of a kinase activity modulating compound or medicament (e.g. a kinase inhibitor). More frequently kinase activity profiles of a sample will be measured by determining the kinase activity of all kinases as taught herein in the same experiment, or in sequentially carried out experiments.

The reference to any marker, including any kinase, peptide, polypeptide, protein, or nucleic acid, corresponds to the marker, kinase, peptide, polypeptide, protein, nucleic acid, commonly known under the respective designations in the art. The terms encompass such markers, kinases, peptides, polypeptides, proteins, or nucleic acids of any organism where found, and particularly of animals, preferably warm-blooded animals, more preferably vertebrates, yet more preferably mammals, including humans and non-human mammals, still more preferably of humans. The terms particularly encompass such markers, kinases, peptides, polypeptides, proteins, or nucleic acids with a native sequence, i.e., ones of which the primary sequence is the same as that of the markers, kinases, peptides, polypeptides, proteins, or nucleic acids found in or derived from nature. A skilled person understands that native sequences may differ between different species due to genetic divergence between such species. Moreover, native sequences may differ between or within different individuals of the same species due to normal genetic diversity (variation) within a given species. Also, native sequences may differ between or even within different individuals of the same species due to post-transcriptional or post-translational modifications. Any such variants or isoforms of markers, kinases, peptides, polypeptides, proteins, or nucleic acids are intended herein. Accordingly, all sequences of markers, kinases, peptides, polypeptides, proteins, or nucleic acids found in or derived from nature are considered “native”. The terms encompass the markers, kinases, peptides, polypeptides, proteins, or nucleic acids when forming a part of a living organism, organ, tissue or cell, when forming a part of a biological sample, as well as when at least partly isolated from such sources.

The terms “sample” or “biological sample” as used throughout this specification in reference to a blood composition obtained (isolated, removed) from a subject. The blood sample as used in present invention is typically obtained (isolated, removed) from a subject (patient) prior to being subjected to a method as taught herein. Blood samples are readily obtainable by minimally invasive methods, and are less unpleasant for a subject than, for example, tissue biopsies. Any suitable weight or volume of a sample may be removed from a subject for analysis.

The present inventors demonstrated that the complex biological host-tumor processes determining the response to ICIs can be captured by the kinase activity profile of peripheral blood mononuclear cells.

Accordingly, in particular embodiments, said blood sample comprises immune cells isolated or enriched from peripheral blood (e.g. peripheral blood mononuclear cells, PBMCs). In particular embodiments, said blood sample is a blood sample comprising peripheral blood monocytes (PBMCs). In particular embodiments, said blood sample is a lysate of blood-derived PBMCs. PBMCs can be isolated from a blood sample by all methods known in the art, such as Ficoll-Isopaque density centrifugation and optionally be preserved by freezing.

In particular embodiments, the PBMCs are isolated from the blood sample at most 96 hours, at most 72 hours, at most 48 hours, at most 36 hours, at most 28 hours, at most 26 hours, at most 24 hours, at most 22 hours, at most 20 hours, at most 18 hours, at most 16 hours, at most 14 hours, at most 12 hours, at most 10 hours, at most 8 hours, at most 6 hours, at most 4 hours, or at most 2 hours, preferably at most 24 hours, after taking the blood sample from the patient.

The PMBCs may be used immediately in the method as taught herein, or may be stored by cryo-preservation for later use. The cryopreserved PBMCs can be easily transported and used in the method as taught herein upon request. In particular embodiments, the total period between taking the blood sample and cryopreserving the PBMCs is at most 96 hours, at most 72 hours, at most 48 hours, at most 36 hours, at most 28 hours, at most 26 hours, at most 24 hours, at most 22 hours, at most 20 hours, at most 18 hours, at most 16 hours, at most 14 hours, at most 12 hours, at most 10 hours, at most 8 hours, at most 6 hours, at most 4 hours, or at most 2 hours, preferably at most 24 hours.

In particular embodiments, the blood sample in which the kinase activity is measured according to the method as taught herein comprises a total amount of at least 1×10⁴ PBMCs, at least 2×10⁴ PBMCs, at least 3×10⁴ PBMCs, at least 4×10⁴ PBMCs, at least 5×10⁴ PBMCs, at least 6×10⁴ PBMCs, at least 7×10⁴ PBMCs, at least 8×10⁴ PBMCs, at least 9×10⁴ PBMCs, or at least 1×10⁶ PBMCs. In particular embodiments, the blood sample in which the kinase activity is measured according to the method as taught herein, comprises a total amount of from 1×10⁴ to 1×10⁶, or from 1×10⁴ to 1×10⁵ PBMCs.

In a preferred embodiment of the present invention said sample is a sample that has undergone a preparation step prior to the steps according to the method of the present invention. Preferably said preparation step is a step where the protein kinases present in said sample are released from the tissue by lysis. The term “tissue” as used herein encompasses all types of cells of the human body including cells of organs but also including blood and other body fluids recited above. Additionally the kinases in the sample may be stabilized, maintained, enriched or isolated, and the measurement of the kinase activity as performed in step (a) occurs on the enriched or isolated protein kinase sample. By first enriching protein kinases in the sample or isolating protein kinases from the sample the subsequent measurement of the kinase activity will occur in a more efficient and reliable manner. Also the clarity and intensity of the obtained kinase activity signal will be increased as certain contaminants are being removed during the enriching or isolating step.

Present inventors observed that the overall kinase activity could be affected by the anticoagulant being used upon collecting the blood sample.

Accordingly, in particular embodiments, said blood sample is not being inhibited from clotting by EDTA. In particular embodiments, said blood sample is inhibited from clotting using citrate, heparin or a salt form or derivative thereof, preferably heparin or a salt form or derivative thereof.

In particular embodiments, said blood sample is inhibited from clotting using sodium heparin, lithium heparin or sodium citrate, preferably sodium heparin.

In particular embodiments, said blood sample is inhibited from clotting using from 10 to 200 IU, from 10 to 100 IU, from 10 to 75 IU, from 10 to 50 IU, from 10 to 30 IU, or from 10 to 20 IU, such as about 17 IU, of sodium heparin or lithium heparin, preferably sodium heparin, per millilitre of blood. The term “international unit” or “IU” as used herein refers to a unit of potency of the sodium heparin or lithium heparin.

The terms “subject”, “individual” or “patient” can be used interchangeably herein, and typically and preferably denote humans, but may also encompass reference to non-human animals, preferably warm-blooded animals, even more preferably mammals, such as, e.g., non-human primates, rodents, canines, felines, equines, ovines, porcines, and the like. The term “non-human animals” includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In certain embodiments, the subject is a non-human mammal. In certain embodiments, the subject is a human subject. The term does not denote a particular age or sex. Thus, adult and new-born subjects, as well as foetuses, whether male or female, are intended to be covered. Examples of subjects include humans, dogs, cats, cows, goats, and mice.

Suitable subjects may include without limitation subjects presenting to a physician for a screening for a neoplastic disease, subjects presenting to a physician with symptoms and signs indicative of a neoplastic disease, subjects diagnosed with a neoplastic disease, subjects who have received anti-cancer therapy, subjects undergoing anti-cancer treatment, and subjects having a neoplastic disease in remission. Methods for diagnosing neoplastic diseases are known in the art and include microscopic analysis of a sample (biopsy) of the affected area of the tissue or organ, such as a skin biopsy. Preferably, said patient in need of an ICI is a patient diagnosed with a neoplastic disease.

The term “patient in need of an immune checkpoint inhibitor” or the “patient in need of an ICI” as used herein has the generally acceptable meaning within the art and preferably refers to a patient diagnosed with a disease or condition, such as a neoplastic disease, for which an ICI, preferably a PD-1 or PD-L1 ICI, has been implicated as a treatment or for which an ICI, preferably a PD-1 or PD-L1 ICI, is a clinically approved therapy. The term does not encompass information on whether the patient would benefit (i.e. responder) or would not benefit (i.e. non-responder) from said ICI, preferably a PD-1 or PD-L1 ICI. In preferred embodiments, said blood sample is obtained from a patient diagnosed with a neoplastic disease, said neoplastic disease preferably being selected from the group consisting of acute myeloid leukaemia, advanced cancer, anal basaloid carcinoma, basaloid squamous cell carcinoma, biliary tract carcinoma, bladder cancer, brain cancer, breast cancer, cervical cancer, clear-cell renal cell carcinoma, colorectal cancer, diffuse large b-cell lymphoma, endometrial cancer, epithelial ovarian cancer, oesophageal cancer, gastric cancer, glioblastoma, head and neck cancer, hepatocellular carcinoma, Hodgkin lymphoma, kidney cancer, liver cancer, lung cancer, malignant melanoma, melanoma, merkel cell carcinoma, mesothelioma, multiple myeloma, non-small cell lung carcinoma, oropharyngeal carcinoma, ovarian cancer, pancreatic cancer, prostate cancer, small cell lung cancer, triple negative breast cancer, and urothelial cancer. The neoplastic disease may or may not be refractory to a first line therapy.

In preferred embodiments, said blood sample is obtained from a patient diagnosed with a neoplastic disease selected from the group consisting of non-small-cell lung carcinoma (NSCLC), bladder cancer, ovarian cancer, prostate cancer, head and neck cancer, colorectal cancer, and melanoma, preferably NSCLC, melanoma, bladder cancer or ovarian cancer, more preferably NSCLC or melanoma.

In particular embodiments, said blood sample is obtained from a patient diagnosed with a metastatic neoplastic disease, such as metastatic NSCLC or metastatic melanoma.

In particular embodiments, said blood sample is obtained from a patient diagnosed with a neoplastic disease selected from the group consisting of stage III NSCLC, stage IV NSCLC, irresectable stage III melanoma, and irresectable stage IV melanoma.

As referred to in the present application NSCLC is regarded as one of the main types of lung cancer and accounts for about 85% of all lung cancers. NSCLC can be further divided into three subtypes, namely squamous cell carcinoma, large cell carcinoma and adenocarcinoma. Adenocarcinoma is the most common type and starts in the mucus making gland cells in the lining of the airways, squamous cell cancer develops in the flat cells that cover the surface of the airways and grows near the centre of the lung and large cell carcinoma appear large and round under the microscope. Other less common types of NSCLC are pleomorphic, carcinoid tumor, salivary gland carcinoma, and unclassified carcinoma.

In particular embodiments, said blood sample is obtained from a patient diagnosed with stage IV adenocarcinoma or squamous cell carcinoma of the lungs.

In particular embodiments, said blood sample is obtained from a patient diagnosed with stage III NSCLC or stage IV NSCLC, preferably stage IV NSCLC.

In particular embodiments, said blood sample is obtained from a patient diagnosed with stage III adenocarcinoma or squamous cell carcinoma of the lungs or stage IV adenocarcinoma or squamous cell carcinoma of the lungs, preferably stage IV adenocarcinoma or squamous cell carcinoma of the lungs.

As referred to in the present application melanoma regards a specific type of skin cancer which forms from melanocytes (pigment-containing cells in the skin). While other types of skin cancer (e.g. basal cell cancer (BCC) and squamous cell cancer (SCC)) are more common, melanoma is considered to be much more dangerous if it is not found in the early stages. It causes the majority (75%) of deaths related to skin cancer. Globally, in 2012, melanoma occurred in 232,000 people and resulted in 55,000 deaths. In particular embodiments, said blood sample is obtained from a patient diagnosed with irresectable stage III or irresectable stage IV melanoma.

In particular embodiments, said blood sample is obtained from a patient diagnosed with a neoplastic disease selected from the group consisting of acute myeloid leukaemia, advanced cancer, anal basaloid carcinoma, basaloid squamous cell carcinoma, biliary tract carcinoma, bladder cancer, brain cancer, breast cancer, cervical cancer, clear-cell renal cell carcinoma, colorectal cancer, diffuse large b-cell lymphoma, endometrial cancer, epithelial ovarian cancer, oesophageal cancer, gastric cancer, glioblastoma, head and neck cancer, hepatocellular carcinoma, Hodgkin lymphoma, kidney cancer, liver cancer, lung cancer, malignant melanoma, melanoma, merkel cell carcinoma, mesothelioma, multiple myeloma, non-small cell lung carcinoma, oropharyngeal carcinoma, ovarian cancer, pancreatic cancer, prostate cancer, small cell lung cancer, triple negative breast cancer, urothelial cancer, and any combination thereof, before onset of treatment with an ICI (e.g. PD-1 or PD-L1 ICI, and/or a combination thereof and/or analogs thereof).

The kinase activity profile of the patient in need of ICI may be compared to a reference kinase activity profile to support in the prediction of the response of said patient to treatment of said PD-1 or PD-1. In particular embodiments, step (b) of predicting from said kinase activity profile the response of said patient to treatment with said PD-1 or PD-L1 ICI comprises a step (i.1) of comparing said kinase activity profile to at least one reference kinase activity profile; said at least one reference kinase activity profile being representative for a good or poor responder to said PD-1 or PD-L1 ICI; and a step (ii.1) of predicting the response of said patient to said PD-1 or PD-L1 ICI on the basis of the comparison of said kinase activity profile with said at least one reference kinase activity profile.

In particular embodiments, step (b) of predicting from said kinase activity profile the response of said patient to treatment with said PD-1 or PD-L1 ICI comprises a step (i.2) of comparing said kinase activity profile to at least one reference kinase activity profile; said at least one reference kinase activity profile representing a known sensitivity (or responsiveness or susceptibility) to said PD-1 or PD-L1 ICI; and a step (ii.2) of predicting the response of said patient to said PD-1 or PD-L1 ICI on the basis of the comparison of said kinase activity profile with said at least one reference kinase activity profile.

For example, a reference kinase activity profile may represent a known sensitivity to treatment with a PD-1 or PD-L1 ICI in the patient, such as the determination that the patient will be sensitive to treatment with a PD-1 or PD-L1 ICI, or the determination that the patient will be resistant to treatment with a PD-1 or PD-L1 ICI. In another example, a reference kinase activity profile may represent responders to treatment with a PD-1 or PD-L1 ICI or non-responders to treatment with a PD-1 or PD-L1 ICI. In yet another example, a reference kinase activity profile may represent a determination of a certain degree of sensitivity to treatment with a PD-1 or PD-L1 ICI in the patient.

In particular embodiments, said at least one reference kinase activity profile represents a known sensitivity (or responsiveness or susceptibility) of a reference subject to treatment with said PD-1 or PD-L1 ICI. In particular embodiments, said at least one reference kinase activity profile may correspond to the kinase activity profile in a blood sample from a reference subject that is sensitive (or responsive, susceptible, or a good responder) to treatment with said PD-1 or PD-L1 ICI. In particular embodiments, said at least one reference kinase activity profile may correspond to the kinase activity profile in a blood sample from a reference subject that is insensitive (or unresponsive, insusceptible, or a poor responder) to treatment with said PD-1 or PD-L1 ICI.

Reference kinase activity profiles may be established according to known procedures. For example, a reference kinase activity profile may be established in a reference subject or individual or a population of individuals characterized by a particular determination of sensitivity to treatment with a PD-1 or PD-L1 ICI (i.e., for whom said determination of sensitivity to treatment a PD-1 or PD-L1 ICI holds true). Such population may comprise without limitation 2 or more, 10 or more, 100 or more, or even several hundred or more individuals. In certain embodiments, the reference subjects are subjects with the same type of neoplastic disease, e.g., to not compare different types of neoplastic diseases, and/or subjects with the same stage of neoplastic disease, e.g., to not compare primary vs. metastatic tumours.

In particular embodiments, step (b) of predicting from said kinase activity profile the response of said patient to treatment with said PD-1 or PD-L1 ICI comprises a step (i.3) of comparing said kinase activity profile to a first and a second reference kinase activity profile; said first reference kinase activity profile being representative for a good responder (or a subject that is responsive or susceptible) to said PD-1 or PD-L1 ICI and said second reference kinase activity profile being representative for a poor responder (or a subject that is unresponsive or insusceptible) to said PD-1 or PD-L1 ICI; and a step (ii.3) of predicting the response of said patient to said PD-1 or PD-L1 ICI on the basis of the comparison of said kinase activity profile with said first and said second reference kinase activity profile.

Accordingly, in particular embodiments, the present invention relates to a method for predicting the response of a patient in need of an ICI to treatment with a PD-1 or PD-L1 ICI, comprising the steps of:

(a) measuring the kinase activity of at least two kinases independently selected from within at least two families of kinases selected from the group consisting of: the VEGFR or PDGFR family of kinases; the SRC family of kinases; the SYK family of kinases; the TAM family of kinases; the JakA family of kinases; and the ALK family of kinases, selected from the group consisting of: the VEGFR or PDGFR family of kinases; the SRC family of kinases; the SYK family of kinases; and the TAM family of kinases, in a blood sample obtained from said patient, thereby providing a kinase activity profile of said blood sample;
(i.1) comparing said kinase activity profile to at least one reference kinase activity profile; said reference kinase activity profile being representative for a good or poor responder to said PD-1 or PD-L1 ICI; and
(ii.1) predicting the response of said patient to said PD-1 or PD-L1 ICI on the basis of the comparison of said kinase activity profile with said at least one reference kinase activity profile.

In particular embodiments, the present invention relates to a method for predicting the response of a patient in need of an ICI to treatment with a PD-1 or PD-L1 ICI, comprising the steps of:

(a) measuring the kinase activity of at least two kinases independently selected from within at least two families of kinases selected from the group consisting of: the VEGFR or PDGFR family of kinases; the SRC family of kinases; the SYK family of kinases; the TAM family of kinases; the JakA family of kinases; and the ALK family of kinases, selected from the group consisting of: the VEGFR or PDGFR family of kinases; the SRC family of kinases; the SYK family of kinases; and the TAM family of kinases, in a blood sample obtained from said patient, thereby providing a kinase activity profile of said blood sample;
(i.3) comparing said kinase activity profile to a first and a second reference kinase activity profile; said first reference kinase activity profile being representative for a good responder to said PD-1 or PD-L1 ICI and said second reference kinase activity profile being representative for a poor responder to said PD-1 or PD-L1 ICI; and
(ii.3) predicting the response of said patient to said PD-1 or PD-L1 ICI on the basis of the comparison of said kinase activity profile with said first and second reference kinase activity profile.

In further particular embodiments, the present invention relates to a method for predicting the response of a patient in need of an ICI to treatment with a PD-1 or PD-L1 ICI, comprising the steps of: (a) measuring the kinase activity (i.e. absence, presence and/or level, preferably level) of at least two kinases independently selected from within at least two families of kinases selected from the group consisting of: the VEGFR or PDGFR family of kinases; the SRC family of kinases; the SYK family of kinases; the TAM family of kinases; the JakA family of kinases; and the ALK family of kinases, preferably selected from the group consisting of: the VEGFR or PDGFR family of kinases; the SRC family of kinases; the SYK family of kinases; and the TAM family of kinases,

in a blood sample obtained from said patient, thereby providing a kinase activity profile of said blood sample;
(i.4) comparing said kinase activity profile to at least one reference kinase activity profile, said at least one reference kinase activity profile representing the kinase activity profile being representative for a good responder or a poor responder to said PD-1 or PD-L1 ICI;
(ii.4) finding a deviation or no deviation of the kinase activity profile as determined in (a) from said at least one reference kinase activity profile; and
(iii.4) attributing said finding of deviation or no deviation to a particular response (or sensitivity) of said patient in need of an ICI to said PD-1 or PD-L1 ICI.

A “deviation” of a first value from a second value may generally encompass any direction (e.g., increase: first value >second value; or decrease: first value <second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In particular embodiments, said deviation may be concluded if said deviation has a statistical significance of p<0.05.

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

In particular embodiments, step (b) of predicting from said kinase activity profile the response of said patient to treatment with said PD-1 or PD-L1 ICI comprises a step (i.5) of calculating a classifier parameter from said kinase activity profile; and a step (ii.5) of predicting the response of said patient to said PD-1 or PD-L1 ICI on the basis of said classifier parameter.

Accordingly, in a particular embodiment, the present invention relates to a method for predicting the response of a patient in need of an ICI to treatment with a PD-1 or PD-L1 ICI, comprising the steps of: (a) measuring the kinase activity of at least two kinases independently selected from within at least two families of kinases selected from the group consisting of: the VEGFR or PDGFR family of kinases; the SRC family of kinases; the SYK family of kinases; and the TAM family of kinases; the JakA family of kinases; the ALK family of kinases, preferably selected from the group consisting of: the VEGFR or PDGFR family of kinases; the SRC family of kinases; the SYK family of kinases; and the TAM family of kinases

in a blood sample obtained from said patient, thereby providing a kinase activity profile of said blood sample;
(i.5) calculating a classifier parameter from said kinase activity profile; and
(ii.5) predicting the response of said patient to said PD-1 or PD-L1 ICI on the basis of said classifier parameter.

By establishing a classifier parameter for determining the prediction of pharmacotherapy response of the patient in need of an ICI the method of the present invention provides a criterion for analysing the results obtained from the method of the present invention. This criterion enables a person to provide a prediction or prognosis on the basis of a single or limited number of data. The person providing the prediction or prognosis does not have to interpret an entire set of data, but rather bases his conclusion on the basis of a single or limited number of criteria.

The term “classifier parameter” as used herein is a discriminating value which has been determined by establishing the kinase activity profile and/or phosphorylation profile of a sample obtained from a patient in need of an ICI, such as a patient suffering from a neoplastic disease as described elsewhere herein. Said discriminating value identifies the prediction of response to pharmacotherapy of patients in need of an ICI. The classifier parameter includes information regarding the activity of several kinases and/or the phosphorylation level of several protein kinase substrates. Classification is a procedure in which individual items are placed into groups based on quantitative information on one or more characteristics inherent in the items (e.g. kinase activity profile of a sample and/or phosphorylation levels or profiles of a sample) and based on a training set of previously labelled items (clinical response to a pharmacotherapy). The classifier parameter is calculated by applying a “classifier” to the measured kinase activity and/or phosphorylation levels of a sample. Based on the classifying parameter a sample is assigned to (or predicted to belong to) a class (predicting the pharmacotherapy response of said patient). The classifier has been previously determined by comparing samples which are known to belong to the respective relevant classes. Several methods are known in the art for developing a classifier including the neural network (Multi-layer Perceptron), support vector machines, k-nearest neighbours, Gaussian mixture model, naive bayes, decision tree, RBF classifiers, random forest, discriminant analysis, linear discriminant analysis, quadratic discriminant analysis, discriminant analysis—principal component analysis, partial least squares discriminant analysis, generalized distance regression and elastic net classification. The classifier parameter determined in this manner is valid for the same experimental setup in future individual tests.

It is not relevant to give an exact threshold value for the classifier parameter. A relevant threshold value can be obtained by correlating the sensitivity and specificity and the sensitivity/specificity for any threshold value. A threshold value resulting in a high sensitivity results in a lower specificity and vice versa. If one wants to increase the positive predictive value of the test to determine whether a patient in need of an ICI will respond to the ICI, then the threshold value of the test can be changed which as a consequence will decrease the negative predictive value of the test to determine whether a patient in need of an ICI will not respond to the ICI. If one wants to increase the negative predictive value of the test to determine whether a patient in need of an ICI will not respond to the ICI, then the threshold value can be changed in the opposite direction which as a consequence will decrease the positive predictive value of the test to determine whether a patient in need of an ICI will respond to the ICI.

It is thus up to the diagnostic engineers to determine which level of positive predictive value/negative predictive value/sensitivity/specificity is desirable and how much loss in positive or negative predictive value is tolerable. The chosen threshold level could be dependent on other diagnostic parameters used in combination with the present method by the diagnostic engineers.

In yet another embodiment, the present invention relates to a method according to the present invention wherein said classifier parameter predicts the response of said patient to said PD-1 or PD-L1 ICI if said classifier parameter is above a first predetermined threshold level, and wherein said classifier parameter indicates non-response to said PD-1 or PD-L1 ICI of said patient if said classifier parameter is below a second predetermined threshold level.

According to another embodiment, the present invention relates to the method of the present invention wherein said differential kinase activity level or said classifier parameter indicates a response, no-response or undetermined or intermediate prediction of said PD-1 or PD-L1 ICI or the effect of the targeted pharmacotherapy of said patient.

In particular embodiments, the measured kinase activity profile is corrected for any variation or changes in the kinase activity measurement set-up including drift, batch effects, or time effects that may have occurred between establishing the reference kinase activity profile, classifying parameter, or classifying model and measuring the kinase activity profile of the patient. The correction may be performed by any methods known in the art. For example, the correction may be performed by use of an internal assay control sample. The internal assay control (IAC) sample may be representative for the blood sample obtained from the patient from who the response to treatment with PD-1 or PD-L1 ICI is being predicted. For example, the IAC sample may be a pooled blood sample comprising PBMCs or a pooled lysate of blood-derived PBMCs obtained from multiple individuals.

In particular embodiments, the method as taught herein comprises:
measuring the kinase activity of at least two kinases independently selected from within at least two families of kinases selected from the group consisting of: the VEGFR or PDGFR family of kinases; the SRC family of kinases; the SYK family of kinases; the TAM family of kinases; the JakA family of kinases; and the ALK family of kinases, preferably selected from the group consisting of the VEGFR or PDGFR family of kinases; the SRC family of kinases; the SYK family of kinases; and the TAM family of kinases; in an IAC sample, thereby providing a kinase activity profile of said IAC sample; and
correcting the kinase activity profile of the patient for any variation in the kinase activity measurement set-up using the kinase activity profile of said IAC sample;
   wherein the kinase activity of the at least two kinases in the IAC sample is measured in parallel with the kinase activity of the at least two kinases in the blood sample obtained from said patient; and optionally, wherein the kinase activity of the at least two kinases in the IAC sample and the kinase activity of the at least two kinases in the blood sample obtained from said patient is measured in series with the kinase activity of the at least two kinases in a reference sample (e.g. of a known good or known poor responder).

The correction of the kinase activity profile of the patient for any variation in the kinase activity measurement set-up using the kinase activity profile of said IAC sample may be performed by
(i) comparing the kinase activity profile of the IAC sample to the kinase activity profile that was measured while establishing the reference kinase activity profile, classifying parameter, or classifying model,
(ii) assessing the difference between the kinase activity profile of the IAC sample and the kinase activity profile that was measured while establishing the reference kinase activity profile, classifying parameter, or classifying model; and
(iii) applying a correction on the kinase activity profile of the patient based on said difference between the IAC sample and the kinase activity profile that was measured while establishing the reference kinase activity profile, classifying parameter, or classifying model.

In particular embodiments, when the kinase activity of the patient's blood sample is determined by contacting the blood sample with at least two protein kinase substrates as listed in Table 2 or 3, the method as taught herein comprises
contacting an IAC sample with at least two, preferably all, protein kinase substrates as listed in Table 2 or Table 3, thereby providing a phosphorylation profile of said IAC sample, said phosphorylation profile comprising the phosphorylation levels of phosphorylation sites present in said at least two, preferably all, protein kinase substrates; and
correcting the kinase activity profile of the patient for any variation in the kinase activity measurement set-up using the phosphorylation profile of said IAC sample;
   wherein the phosphorylation profile of said IAC sample is measured in parallel with the phosphorylation profile of the blood sample obtained from said patient.

The correction of the kinase activity profile of the patient for any variation in the kinase activity measurement set-up using the phosphorylation profile of said IAC sample may be performed by
(i) comparing the phosphorylation profile of the IAC sample to the phosphorylation profile that was measured while establishing the reference kinase activity profile, classifying parameter, or classifying model,
(ii) assessing the difference between the phosphorylation profile of the IAC sample and the phosphorylation profile that was measured while establishing the reference kinase activity profile, classifying parameter, or classifying model; and
(iii) applying a correction on the kinase activity profile of the patient based on said difference between the IAC sample and the phosphorylation profile that was measured while establishing the reference kinase activity profile, classifying parameter, or classifying model.

Multiple methods may be applied to perform the correction of the kinase activity profile of the patient, including but not limited to, a univariate multiplication by or subtraction of the observed difference, a multivariate method, such as a multivariate model aiming at reducing the noise associated with the correction by considering the phosphorylation of multiple protein kinase substrates simultaneously. Multivariate methods may include principal component analysis/latent variable based methods including Dynamic Orthogonal Projection (P. Gujral, M. Amrhein, and D. Bonvin, “Drift correction in multivariate calibration models using on-line reference measurements,” Anal. Chim. Acta, vol. 642, no. 1-2, pp. 27-36, May 2009, P. Gujral, M. Amrhein, B. M. Wise, and D. Bonvin, “Special Issue Article Framework for explicit drift correction in multivariate calibration models,” 2010, M. Dabros, M. Amrhein, P. Gujral, and U. von Stockar, “On-Line Recalibration of Spectral Measurements Using Metabolite Injections and Dynamic Orthogonal Projection,” Appl. Spectrosc., vol. 61, no. 5, pp. 507-513, May 2007.) or Empirical Bayes methods such as ComBat ((W. E. Johnson, C. Li, and A. Rabinovic, “Adjusting batch effects in microarray expression data using empirical Bayes methods,” Biostatistics, vol. 8, no. 1, pp. 118-127, January 2007).

In particular embodiments, the kinase activity of the at least two kinases in the blood sample obtained from said patient is measured in parallel with the kinase activity of the at least two kinases in a reference sample (e.g. of a known good or known poor responder).

In more particular embodiments, the kinase activity profile as determined in (a) indicates a poor response of said patient to said PD-1 or PD-L1 ICI if
the kinase activity of a kinase of the VEGFR or PDGFR family of kinases, preferably selected from the group consisting of VEGFR1, VEGFR2, VEGFR3, PDGFRalpha, PDGFRbeta, CSF-1R, Kit, and FLT3, preferably VEGFR1, VEGFR2, VEGFR3, PDGFRalpha, PDGFRbeta, and FLT3, is lower than or equal to a reference kinase activity of said kinase representing a poor responder;
the kinase activity of a kinase of the TAM family of kinases, preferably selected from the group consisting of TYRO3, AXL and MERTK, is lower than or equal to a reference kinase activity of said kinase representing a poor responder;
the kinase activity of a kinase of the SRC family of kinases, preferably selected from SRC, YES, FYN, FGR, LCK, HCK, BLK, LYN, and FRK, is higher than or equal to a reference kinase activity of said kinase representing a poor responder;
the kinase activity of a kinase of the SYK family of kinases, preferably selected from SYK and ZAP-70, is higher than or equal to a reference kinase activity of said kinase representing a poor responder;
the kinase activity of RON is higher than or equal to a reference kinase activity of said kinase representing a poor responder; and/or
the kinase activity of ALK is higher than or equal to a reference kinase activity of said kinase representing a poor responder.

In particular embodiments, the kinase activity profile as determined in (a) indicates a good response of said patient to said PD-1 or PD-L1 ICI if
the kinase activity of a kinase of the VEGFR or PDGFR family of kinases, preferably selected from VEGFR1, VEGFR2, VEGFR3, PDGFRalpha, PDGFRbeta, CSF-1R, Kit, and FLT3, preferably VEGFR1, VEGFR2, VEGFR3, PDGFRalpha, PDGFRbeta, and FLT3, is higher than or equal to a reference kinase activity of said kinases representing a good responder;
the kinase activity of a kinase of a kinase of the TAM family of kinases, preferably selected from TYRO3, AXL, and MERTK, is higher than or equal to a reference kinase activity of said kinases representing a good responder;
the kinase activity of a kinase of the SRC family of kinases, preferably selected from SRC, YES, FYN, FGR, LCK, HCK, BLK, LYN and FRK, more preferably selected from SRC, YES, FYN, LCK, HCK, BLK, LYN, and FRK, is lower than or equal to a reference kinase activity of said kinases representing a good responder;
the kinase activity of a kinase of the SYK family of kinases, preferably selected from SYK and ZAP-70, is lower than or equal to a reference kinase activity of said kinase representing a good responder;
the kinase activity of RON is lower than or equal to compared to a reference kinase activity of said kinase representing a good responder; and/or
the kinase activity of ALK is lower than or equal to compared to a reference kinase activity of said kinase representing a good responder.

In more particular embodiments, the kinase activity profile as determined in (a) indicates a poor response of said patient to said PD-1 or PD-L1 ICI if
the kinase activity of a kinase of the VEGFR or PDGFR family of kinases, preferably selected from the group consisting of VEGFR1, VEGFR2, VEGFR3, PDGFRalpha, PDGFRbeta, CSF-1R, Kit, and FLT3, is higher than the classifier parameter;
the kinase activity of a kinase of the TAM family of kinases, preferably selected from the group consisting of TYRO3, AXL and MERTK, is higher than the classifier parameter;
the kinase activity of a kinase of the SRC family of kinases, preferably selected from SRC, YES, FYN, FGR, LCK, HCK, BLK, LYN, and FRK, is lower than the classifier parameter;
the kinase activity of a kinase of the SYK family of kinases, preferably selected from SYK and ZAP-70, is lower than the classifier parameter;
the kinase activity of a kinase of the JakA family of kinases, preferably selected from JAK1, JAK2, JAK3, TYK2, more preferably JAK1, is higher than the classifier parameter;
the kinase activity of a kinase of the ALK family of kinases, preferably selected from ALK and TLK, is lower than the classifier parameter; and/or
the kinase activity of RON is lower than the classifier parameter.

As used in the present application the prediction of response to a PD-1 or PD-L1 ICI of patients in need of an ICI is generally divided into two types of non-responders and responders and additionally some undetermined or intermediate responders. Whereas responders to a PD-1 or PD-L1 ICI will survive longer or have additional clinical benefits (e.g. improved quality of life, prolonged progression free survival, etc.) due to the treatment, the non-responders will not benefit from the PD-1 or PD-L1 ICI. The method of the present invention specifically enables the distinction between responders (e.g. clinical benefit such as complete response (CR), partial response (PR), stable disease (SD)) and non-responders (e.g. no clinical benefit such as progressive disease (PD)) to a PD-1 or PD-L1 ICI. The method of the present invention also enables the distinction between patients with an early (e.g. <140 days after initiation of therapy) and late (e.g. >140 days after initiation of therapy) progression of disease upon treatment with a PD-1 or PD-L1 ICI.

The kinase activity of at least two kinases independently selected from within at least two families of kinases selected from the group consisting of the VEGFR or PDGFR family of kinases; the SRC family of kinases; the SYK family of kinases; and the TAM family of kinases; the JakA family of kinases; the ALK family of kinases, and optionally at least one kinases of the group consisting of TRKC, RON, and any combination thereof, may be determined by any means known in the art to determine kinase activity. For measuring the kinase activity of the sample a large variety of methods and formats are known in the art.

The activation state of a kinase and/or a substantial fraction of the entire kinome can be measured using, for example, macroarrays, microarrays, antibody-based arrays, mass spectrometry (MS), reverse-phase protein arrays, kinase activity assay for kinome profiling (KAYAK) methodology, the KiNativ platform, bead arrays (e.g. kinobeads), PamChip method, Pepscan Presto method, ELISA and multiplex ELISA techniques, blotting methods, surface plasmon resonance, capillary electrophoresis and FACS based cell sorting. More particularly, antibody-based arrays may be used to determine the level of phosphorylated proteins and protein kinases; mass spectrometry (MS)-based approaches can be used to investigate the activity of the kinome; reverse-phase protein arrays include arrays which use cellular lysates that are immobilized on an array platform and which then are probed with specific phospho-antibodies; kinase activity assay for kinome profiling (KAYAK) methodology may use known substrate preferences of various protein kinases that are dictated by motifs surrounding the site of phosphorylation; the KiNativ platform may use specific beads to pull down kinases which can be combined with MS; kinobeads (i.e. beads linked to kinase inhibitors) can act as traps for activated kinases present in the samples; the PamChip method can profile activity of kinases using a flow-through peptide-microarray platform; the Pepscan Presto method can use peptide immobilized to glass surfaces and detection using radioactive 33p incorporation based on activity of kinases; ELISA formats can allow for high-throughput screening of activity of kinases using immobilized phosphospecific antibodies in kinase inhibitors; and FACS based cell sorting combined with intracellular probes can identify phosphorylated proteins and protein kinases.

In particular embodiments, said kinase activity is determined by contacting the blood sample with at least one protein kinase substrate, thereby providing a phosphorylation profile of said blood sample. In more particular embodiments, said kinase activity is determined by contacting the blood sample with at least one protein kinase substrate, thereby providing a phosphorylation profile of said blood sample, said phosphorylation profile comprising the phosphorylation levels of phosphorylation sites present in all 142 peptide markers as listed in Table 2 or all 62 peptide markers as listed in Table 3, preferably all 62 peptide markers as listed in Table 3.

As used in the present invention, the term “phosphorylation profile” refers to a data set representative for the phosphorylation levels of, preferably one or more, phosphorylation sites present on the protein kinase substrates. If the kinase activity of the kinases as taught herein is determined by contacting the blood sample with at least one protein kinase substrate a specific phosphorylation profile is obtained. The phosphorylation profile is generated by the phosphorylation of the protein kinase substrates with the protein kinases present in the blood sample and it comprises the level of phosphorylation of the phosphorylation sites present on the protein kinase substrates used. A phosphorylation profile can thus be generated when using at least one protein kinase substrate in different test conditions such as for example by comparing the phosphorylation of a sample on one peptide or protein (protein kinase substrate) in the presence and absence of a phosphatase modulating compound or PD-1 or PD-L1 ICI. More frequently phosphorylation profiles of a blood sample will be measured using several protein kinase substrates in the same or sequentially carried out experiments. Preferably, the present invention determines tyrosine, serine and threonine kinase activity levels or profiles. Most preferably, the present invention determines tyrosine kinase activity levels or profiles.

It should be noted that a person skilled in the art will appreciate that the methods of the present invention can use phosphorylation profiles as a basis for determining protein kinase activity. However, the phosphorylation levels of individual protein kinase substrates can also be used as a basis for determining protein kinase activity.

The person skilled in the art will understand how to determine from said phosphorylation profile of said sample a kinase activity profile reflecting the kinase activity of at least two kinases independently selected from within at least two families of kinases selected from the group consisting of: the VEGFR or PDGFR family of kinases; the SRC family of kinases; the SYK family of kinases; the TAM family of kinases; the JakA family of kinases; the ALK family of kinases; and optionally at least one kinases of the group consisting of TRKC, RON, and any combination thereof.

For example, the kinase activity profile can be determined from said phosphorylation profile of said sample using upstream kinase analysis. This may be achieved by using information from knowledge databases (e.g. HRPD, PhosphoSite, Reactome and PhosphoNET).

In particular embodiments, in step (a) said kinase activity is determined by contacting the blood sample with at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, or all 142 of the protein kinase substrates as listed in Table 2; or with at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, or all 62 of the protein kinase substrates as listed in Table 3, thereby providing a phosphorylation profile of said blood sample, said phosphorylation profile comprising the phosphorylation levels of phosphorylation sites present in said at least two protein kinase substrates.

In preferred embodiments, in step (a) said kinase activity is determined by contacting the blood sample with at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, or all 62 of the protein kinase substrates as listed in Table 3, thereby providing a phosphorylation profile of said blood sample, said phosphorylation profile comprising the phosphorylation levels of phosphorylation sites present in said at least 2 protein kinase substrates. Similar to the classifier parameter calculated from the kinase activity as taught herein, a classifier parameter can also be calculated from the phosphorylation levels of a sample. These classifier parameters calculated from the phosphorylation levels of a sample can be used to determine the kinase activity from the phosphorylation profile.

It should be noted that for the measurement of the protein kinase activity, ATP or any other phosphate source needs to be added to the sample when it is contacted with the protein kinase substrates. The presence of ATP will lead to a phosphorylation of the protein kinase substrates. Alternatively, the phosphorylation of the protein kinase substrates can be performed in the absence of exogenous ATP. When no ATP is added during the incubation of the sample with the protein kinase substrates, the endogenous ATP, the ATP naturally present in the sample, will act as the primary source of ATP.

The phosphorylation level of each of the protein kinase substrates can be monitored using any method known in the art. The response of the protein kinase substrates is determined using a detectable signal, said signal resulting from the interaction of the sample with the protein kinase substrates or by for instance measuring mass differences using mass spectrometry. In determining the interaction of the sample with the protein kinase substrates the signal is the result of the interaction of the phosphorylated substrates with a molecule capable of binding to the phosphorylated substrates. This binding can be detected by e.g. surface plasmon resonance or by the molecule being detectably labelled. For the latter, the molecule that specifically binds to the substrates of interest (e.g. antibody or polynucleotide probe) can be detectably labelled by virtue of containing an atom (e.g. radionuclide), molecule (e.g. fluorescein), or enzyme or particle or complex that, due to a physical or chemical property, indicates the presence of the molecule. A molecule may also be detectably labelled when it is covalently bound to or otherwise associated with a “reporter” molecule (e.g. a biomolecule such as an enzyme) that acts on a substrate to produce a detectable atom, molecule or other complex. Detectable labels suitable for use in the present invention include any composition detectable by spectroscopic, photochemical, biochemical, immunochemical, electrical, optical or chemical means. Labels useful in the present invention include biotin for staining with labelled avidin or streptavidin conjugate, magnetic beads (e.g. Dynabeads'), fluorescent dyes (e.g. fluorescein, fluorescein-isothiocyanate (FITC), Texas red, rhodamine, green fluorescent protein, enhanced green fluorescent protein and related proteins with other fluorescence emission wavelengths, lissamine, phycoerythrin, Cy2, Cy3, Cy3.5, Cy5, Cy5.5, Cy7, FluorX [Amersham], SYBR Green I & II [Molecular Probes], and the like), radiolabels (e.g. 3H, 125I, 35S, 14C, or 32P), enzymes (e.g. luciferases, hydrolases, particularly phosphatases such as alkaline phosphatase, esterases and glycosidases, or oxidoreductases, particularly peroxidases such as horse radish peroxidase, and the like), substrates, cofactors, chemilluminescent groups, chromogenic agents, and colorimetric labels such as colloidal gold or coloured glass or plastic (e. g. polystyrene, polypropylene, latex, etc.), protein particles or beads. In particular, all detectable labels well known to those skilled in the art may be used as detectable labels for use in the present invention.

Means of detecting such labels are well known to those of skill in the art. Thus, for example, chemiluminescent and radioactive labels may be detected using photographic film or scintillation counters, and fluorescent markers may be detected using a photodetector to detect emitted light (e.g. as in fluorescence-activated cell sorting). Enzymatic labels are typically detected by providing the enzyme with a substrate and detecting a coloured reaction product produced by the action of the enzyme on the substrate. Colorimetric labels are detected by simply visualizing the coloured label. Thus, for example, where the label is a radioactive label, means for detection include a scintillation counter, photographic film as in autoradiography, or storage phosphor imaging. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Also, simple colorimetric labels may be detected by observing the colour associated with the label. Fluorescence resonance energy transfer has been adapted to detect binding of unlabeled ligands, which may be useful on arrays.

In a particular embodiment of the present invention the response of the protein kinase substrates to the sample is determined using detectably labelled antibodies; more in particular fluorescently labelled antibodies. In those embodiments of the invention where the substrates consist of protein kinase substrates, the response of the protein kinase substrates is determined using fluorescently labelled anti-phosphotyrosine antibodies, fluorescently labelled anti-phosphoserine or fluorescently labelled anti-phosphothreonine antibodies. The use of fluorescently labelled anti-phosphotyrosine antibodies or fluorescently labelled anti-phosphoserine or fluorescently labelled anti-phosphothreonine antibodies in the method of the present invention, allows real-time or semi real-time determination of the protein kinase activity and accordingly provides the possibility to express the protein kinase activity as the initial velocity of protein kinase derived from the activity over a certain period of incubation of the sample on the substrates.

The term “peptide markers” in the context of the present invention refers to the fact that the peptides as listed in Table 2 or Table 3 can be preferably used according to the methods of the present invention to measure the phosphorylation levels of phosphorylation sites of said markers in blood sample. The phosphorylation levels of the individual phosphorylation sites present in said markers may be measured and compared in different ways. Therefore the present invention is not limited to the use of peptides identical to any of these peptide markers as listed in Table 2 or Table 3 as such. The skilled person may easily on the basis of the peptide markers listed in Table 2 or Table 3 design variant peptides compared to the specific peptides in said Table and use such variant peptides in a method for measuring phosphorylation levels of phosphorylation sites common to said peptide markers as listed in Table 2 or Table 3. These variant peptides may have one or more (2, 3, 4, 5, 6, 7, etc.) amino acids more or less than the given peptides and may also have amino acid substitutions (preferably conservative amino acid substitutions) as long as these variant peptides retain at least one or more of the phosphorylation sites of said original peptides as listed in said tables. Further the skilled person may also easily carry out the methods according to the present invention by using proteins (full length or N- or C-terminally truncated) comprising the amino acid regions of the “peptide markers” listed in Table 2 or Table 3 as sources for studying the phosphorylation of sites present in the amino acid regions of the peptides listed in Table 2 or Table 3. Also the skilled person may use peptide mimetics.

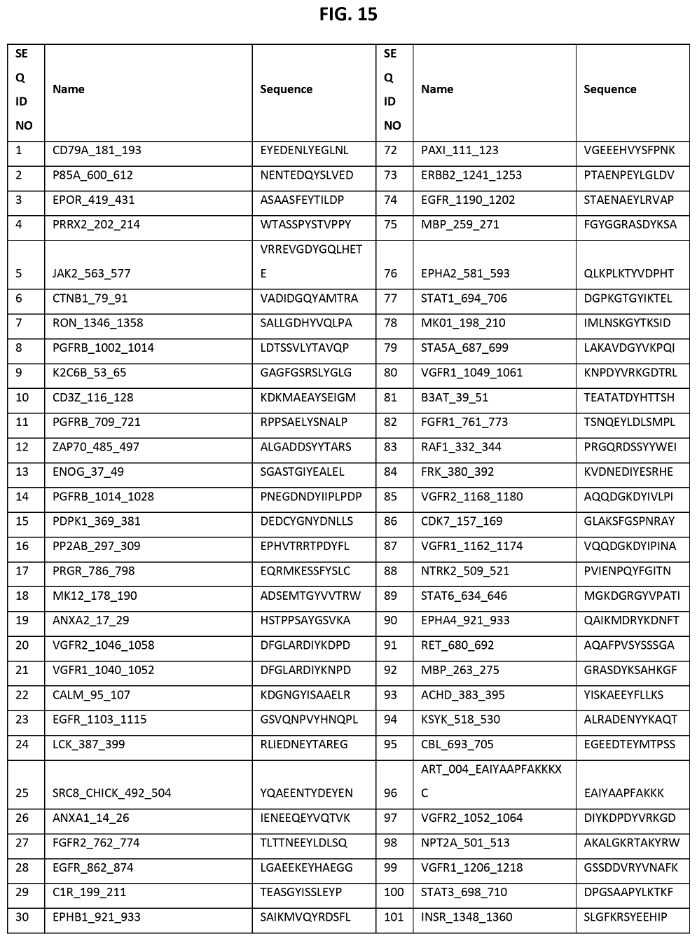

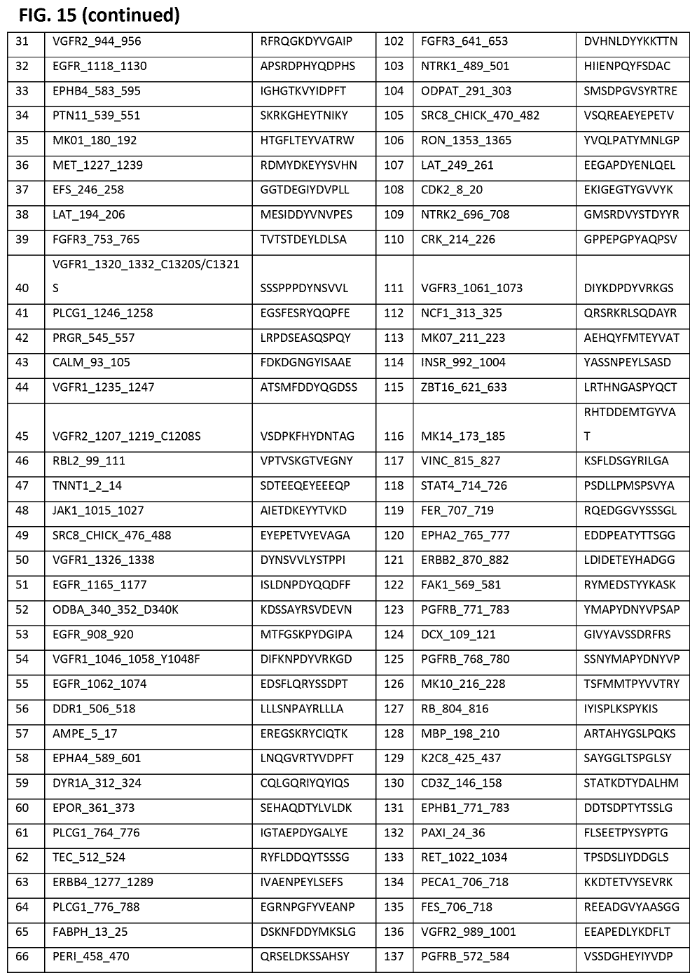

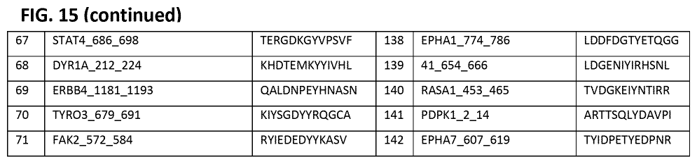

The protein kinase substrates as used in the methods described herein, are meant to include peptides, proteins or peptide mimetics comprising, preferably one or more, of the phosphorylation sites of the peptide markers of Table 2 (see FIG. 15) or Table 3. Said one or more phosphorylation sites are specifically phosphorylated by the protein kinases present in the sample thereby providing a phosphorylation profile. More preferably the protein kinase substrates (peptides, proteins or peptide mimetics) as used in the method of the present invention comprise or consist of all of the peptide markers listed in Table 2 or Table 3, preferably Table 3.

TABLE 3
list of 62 preferred peptide markers comprising phosphorylation
sites used for determining the kinase activity of the at least
two families of kinases selected from the group consisting of
the VEGFR or PDGFR family of kinases; the SRC family of kinases;
the SYK family of kinases; and the TAM family of kinases; the
JakA family of kinases; the ALK family of kinases, and optionally
the family of kinases consisting of TRKC, RON, and any combination
thereof, their sequence and SEQ ID NO. The name of the peptide
markers refers to the associated proteins and to the start and
the end position of the amino acid sequence.

| SEQ ID NO | Name |
|---|---|
| 133 | RET_1022_1034 |
| 18 | MK12_178_190 |
| 134 | PECA1_706_718 |
| 113 | MK07_211_223 |
| 117 | VINC_815_827 |
| 86 | CDK7_157_169 |
| 126 | MK10_216_228 |
| 46 | RBL2_99_111 |
| 69 | ERBB4_1181_1193 |
| 115 | ZBT16_621_633 |
| 12 | ZAP70_485_497 |
| 131 | EPHB1_771_783 |
| 119 | FER_707_719 |
| 19 | ANXA2_17_29 |
| 118 | STAT4_714_726 |
| 84 | FRK_380_392 |
| 111 | VGFR3_1061_1073 |
| 48 | JAK1_1015_1027 |
| 124 | DCX_109_121 |
| 108 | CDK2_8_20 |
| 114 | INSR_1348_1360 |
| 75 | MBP_259_271 |
| 93 | ACHD_383_395 |
| 104 | ODPAT_291_303 |
| 24 | LCK_387_399 |
| 136 | VGFR2_989_1001 |
| 28 | EGFR_862_874 |
| 138 | EPHA1_774_786 |
| 47 | TNNT1_2_14 |
| 5 | JAK2_563_577 |
| 23 | EGFR_1103_1115 |
| 3 | EPOR_419_431 |
| 34 | PTN11_539_551 |
| 123 | PGFRB_771_783 |
| 10 | CD3Z_116_128 |
| 59 | DYR1A_312_324 |
| 135 | FES_706_718 |
| 60 | EPOR_361_373 |
| 58 | EPHA4_589_601 |
| 31 | VGFR2_944_956 |
| 4 | PRRX2_202_214 |
| 80 | VGFR1_1049_1061 |
| 112 | NCF1_313_325 |
| 110 | CRK_214_226 |
| 120 | EPHA2_765_777 |
| 16 | PP2AB_297_309 |
| 29 | C1R_199_211 |
| 81 | B3AT_39_51 |
| 9 | K2C6B_53_65 |
| 127 | RB_804_816 |
| 17 | PRGR_786_798 |
| 142 | EPHA7_607_619 |
| 46 | RBL2_99_111 |
| 36 | MET_1227_1239 |
| 70 | TYRO3_679_691 |
| 35 | MK01_180_192 |
| 72 | PAXI_111_123 |
| 99 | VGFR1_1206_1218 |
| 6 | CTNB1_79_91 |
| 51 | EGFR_1165_1177 |
| 33 | EPHB4_583_595 |
| 2 | P85A_600_612 |

It should further be noted that according to a preferred embodiment of the present invention the peptide markers as listed in Table 2 or Table 3 can be used as such for carrying out the methods according to the present invention. The present invention however also includes the use of analogs and combinations of these peptide markers for use in the method according to the present invention. The peptide marker analogs include peptide markers which show a sequence identity of more than 70%, preferably more than 80%, more preferably more than 90% and even more preferably more than 95%, such as 96%, 97%; 98% or 99% sequence identity.

The medicament as used in the method of the present invention is a PD-1 immune checkpoint inhibitor or a PD-L1 immune checkpoint inhibitor. As used herein, the term “immune checkpoint” refers to an inhibitory pathways hardwired into the immune system that is crucial for maintaining self-tolerance and modulating the duration and amplitude of physiological immune responses in peripheral tissues in order to minimize collateral tissue damage. Tumors can designate one or multiple immune-checkpoint pathways as a major mechanism of immune resistance, particularly against T cells that are specific for tumor antigens. Immune checkpoints can be blocked by antibodies. PD-1 and PD-L1 are such immune checkpoints. Non-limiting examples of PD-1 ICIs include pembrolizumab, nivolumab, cemiplimab, spartalizumab, camrelizumab, sintilimab, tislelizumab, toripalimab, dostarlimab, INCMGA00012, AMP-224 and AMP-514. Non-limiting examples of PD-L1 ICIs include atezolizumab, avelumab, durvalumab, KN035, CK-301, AUNP12, CA-170 and BMS-986189. Specifically said PD-1 or PD-L1 ICI can be an immunotherapeutic antibody directed against PD-1 (such as Nivolumab (e.g. Opdivo™), Pembrolizumab (e.g. Keytruda™), or Durvalumab (e.g. Imfinzi™)) or an immunotherapeutic antibody directed against PD-L1 (such as Atezolizumab (e.g. Tecentriq™), Avelumab (e.g. Bacencio™) and Cemiplimab (e.g. Libtayo™)).

In particular embodiments, the PD-1 or PD-L1 ICI is a protein, polypeptide, peptide, a small molecule, an antibody, an antibody fragment, or any combination thereof, preferably the PD-1 or PD-L1 ICI is an antibody.

The term “protein” as used throughout this specification generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native proteins, such as, e.g., amino acid deletions, additions and/or substitutions. The term contemplates both full-length proteins and protein parts or fragments, e.g., naturally-occurring protein parts that ensue from processing of such full-length proteins.

The term “polypeptide” as used throughout this specification generally encompasses polymeric chains of amino acid residues linked by peptide bonds. Hence, especially when a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally-occurring polypeptide parts that ensue from processing of such full-length polypeptides.

The term “peptide” as used throughout this specification preferably refers to a polypeptide as used herein consisting essentially of 50 amino acids or less, e.g., 45 amino acids or less, preferably 40 amino acids or less, e.g., 35 amino acids or less, more preferably 30 amino acids or less, e.g., 25 or less, 20 or less, 15 or less, 10 or less or 5 or less amino acids.

The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da.

As used herein, the term “antibody” is used in its broadest sense and generally refers to any immunologic binding agent. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest, i.e., antigen-binding fragments), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro or in vivo.

An antibody may be any of IgA, IgD, IgE, IgG and IgM classes, and preferably IgG class antibody. An antibody may be a polyclonal antibody, e.g., an antiserum or immunoglobulins purified there from (e.g., affinity-purified). An antibody may be a monoclonal antibody or a mixture of monoclonal antibodies. Monoclonal antibodies can target a particular antigen or a particular epitope within an antigen with greater selectivity and reproducibility. By means of example and not limitation, monoclonal antibodies may be made by the hybridoma method first described by Kohler et al. 1975 (Nature 256: 495), or may be made by recombinant DNA methods (e.g., as in U.S. Pat. No. 4,816,567). Monoclonal antibodies may also be isolated from phage antibody libraries using techniques as described by Clackson et al. 1991 (Nature 352: 624-628) and Marks et al. 1991 (J Mol Biol 222: 581-597), for example.

Antibody binding agents may be antibody fragments. “Antibody fragments” comprise a portion of an intact antibody, comprising the antigen-binding or variable region thereof. Examples of antibody fragments include Fab, Fab′, F(ab′)2, Fv and scFv fragments, single domain (sd) Fv, such as VH domains, VL domains and VHH domains; diabodies; linear antibodies; single-chain antibody molecules, in particular heavy-chain antibodies; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse.

In certain embodiments, the agent may be a Nanobody®. The terms “Nanobody®” and “Nanobodies®” are trademarks of Ablynx NV (Belgium). In particular embodiments, the PD-1 or PD-L1 ICI is an antibody, preferably selected from the group consisting of Nivolumab, Pembrolizumab, Durvalumab, Atezolizumab, Avelumab, Cemiplimab, Camrelizumab, Sintilimab, Tislelizumab, Toripalimab, and any combination thereof and/or analogue thereof, preferably Nivolumab or Prembrolizumab.

In particular embodiments, the treatment with the PD-1 or PD-L1 inhibitor, preferably Nivolumab or Prembrolizumab, is a combination treatment with the PD-1 or PD-L1 inhibitor, preferably Nivolumab or Prembrolizumab, and with a CTLA-4 ICI, preferably Ipilimumab or Tremilimumab. Accordingly, in particular embodiments, the treatment comprises the administration of the PD-1 or PD-L1 inhibitor, preferably Nivolumab or Prembrolizumab, in combination with a CTLA-4 ICI, preferably Ipilimumab or Tremilimumab. For example, the treatment comprises the administration of Nivolumab in combination with Ipilimumab.

In particular embodiments, the PD-1 or PD-L1 ICI is an immunotherapeutic antibody. In particular embodiments, the PD-1 or PD-L1 ICI is a monoclonal antibody. As used herein, the term “immunotherapeutic antibody” refers to a type of antibody, preferably a monoclonal antibody, which binds to a specific cell or protein, preferably a cell surface protein, and thereby stimulates the immune system to attack those cells. The immunotherapeutic antibody is used in the treatment, cure, prevention, or diagnosis of disease or used to otherwise enhance physical or mental well-being.

In particular embodiment, the treatment with a PD-1 or PD-L1 ICI is combined with an antineoplastic treatment selected from the group consisting of chemotherapy, radiotherapy, chemoradiotherapy, an ICI (preferably different from the PD-1 or PD-L1 ICI, such as a CTLA-4 ICI), a kinase inhibitor, a vaccine, an antibody-drug conjugate, a nuclear receptor agonist, a nuclear receptor antagonist, a cytokine modulator, a chemokine modulator, and any combination thereof.

In particular embodiment, the treatment with a PD-1 or PD-L1 ICI is a combination treatment comprising the treatment with a PD-1 or PD-L1 ICI and the treatment with an antineoplastic treatment selected from the group consisting of chemotherapy, radiotherapy, chemoradiotherapy, an ICI (preferably different from the PD-1 or PD-L1 ICI, such as a CTLA-4 ICI), surgery, a kinase inhibitor, a vaccine, an antibody-drug conjugate, a nuclear receptor agonist, a nuclear receptor antagonist, a cytokine modulator, a chemokine modulator, and any combination thereof.

In particular embodiment, the treatment with a PD-1 or PD-L1 ICI is a combination treatment comprising the treatment with a PD-1 or PD-L1 ICI and the treatment with a CTLA-4 ICI, preferably Ipilimumab or Tremilimumab.

The term “chemotherapy” as used herein is conceived broadly and generally encompasses treatments using chemical substances or compositions. Chemotherapeutic agents may typically display cytotoxic or cytostatic effects. Non-limiting examples of chemotherapy agents include alkylating agents (e.g. altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolamide, thiotepa, trabectedin), nitrosoureas (e.g. carmustine, lomustine, streptozocin), plant alkaloids, antimetabolites (e.g. azacitidine, 5-fluorouracil, 6-mercaptopurine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, trifluridine/tipiracil combination), anti-tumour antibiotics (e.g. daunorubicin, doxorubicin, doxorubicin liposomal, epirubicin, idarubicin, valrubicin, dactinomycin, bleomycin, mitomycin-c, mitoxantrone), topoisomerase inhibitors (e.g. etoposide, mitoxantrone, teniposide, irinotecan, irinotecan liposomal, topotecan), corticosteroids (e.g. prednisone, methylprednisolone, dexamethasone) and mitotic inhibitors (e.g. docetaxel, cabazitaxel, nab-paclitaxel, paclitaxel, vinblastine, vincristine, vincristine liposomal, vinorelbine).

In certain embodiments, a chemotherapeutic agent may be an alkylating agent, a cytotoxic compound, an anti-metabolite, a plant alkaloid, a terpenoid, a topoisomerase inhibitor, or a combination thereof. In certain embodiments, a chemotherapeutic agent may be selected from the group consisting of cyclophosphamide, doxorubicin, idarubicin, mitoxantrone, oxaliplatin, bortezomib, digoxin, digitoxin, hypericin, shikonin, wogonin, sorafenib, everolimus, imatinib, geldanamycin, panobinostat, carmustine, cisplatin, carboplatin, mechlorethamine, melphalan (hydrochloride), chlorambucil, ifosfamide, busulfan, actinomycin, daunorubicin, valrubicin, epirubicin, bleomycin, plicamycin, mitoxantrone, mitomycin, azathioprine, mercaptopurine, fluorouracil, methotrexate, nelarabine, pemetrexed, vincristine, vinblastine, vinorelbine, vindesine, paclitaxel, docetaxel, irinotecan, topotecan, amsacrine, etoposide, etoposide phosphate, teniposide, anastrozole, exemestane, bosutinib, irinotecan, vandetanib, bicalutamide, lomustine, clofarabine, cabozantinib, cytarabine, cytoxan, decitabine, dexamethasone, hydroxyurea, decarbazine, leuprolide, epirubicin, asparaginase, estramustine, vismodegib, amifostine, flutamide, toremifene, fulvestrant, letrozole, degarelix, fludarabine, pralatrexate, floxuridine, gemcitabine, carmustine wafer, eribulin, altretamine, topotecan, axitinib, gefitinib, romidepsin, ixabepilone, ruxolitinib, cabazitaxel, carfilzomib, chlorambucil, sargramostim, cladribine, leuprolide, mitotane, procarbazine, megestrol, mesna, strontium-89 chloride, mitomycin, filgrastim, pegfilgrastim, sorafenib, nilutamide, pentostatin, tamoxifen, pegaspargase, denileukin diftitox, alitretinoin, carboplatin, prednisone, mercaptopurine, zoledronic acid, lenalidomide, octreotide, dasatinib, regorafenib, histrelin, sunitinib, omacetaxine, thioguanine, erlotinib, bexarotene, decarbazine, temozolomide, thiotepa, thalidomide, BCG, temsirolimus, bendamustine hydrochloride, triptorelin, arsenic trioxide, lapatinib, valrubicin intravesical, tretinoin, azacitidine, pazopanib, teniposide, leucovorin, crizotinib, capecitabine, enzalutamide, ziv-aflibercept, streptozocin, vemurafenib, goserelin, vorinostat, zoledronic acid, abiraterone, and combinations thereof.

The term “surgery” as used throughout this specification broadly denotes treatments comprising surgical removal of neoplastic tissue or cells from a subject. Cancer surgery may remove an entire tumor, debulk a tumor, or remove a tumor or a portion thereof causing pain or pressure. Cancer surgery includes inter alia conventional open surgery, laparoscopic surgery, cryosurgery, laser surgery, thermal ablation such as hyperthermic laser ablation or radiofrequency ablation, photodynamic therapy, and combinations thereof.

The term “radiotherapy” as used throughout this specification broadly denotes treatments comprising the exposure of neoplastic tissue to ionizing radiation, such as radiation from x-rays, gamma rays, neutrons, protons, or other sources. The source of the radiation may be an external apparatus (external-beam radiation therapy), or the radioactive material may be placed in the body near the neoplastic tissue (internal radiation therapy or brachytherapy), or radioactive material may be delivered systemically by injection, infusion or ingestion (systemic radioisotope therapy) and may concentrate in the neoplastic tissue spontaneously or by means of a targeting moiety, such as a cancer-targeting antibody.

The term “chemoradiotherapy” as used herein refers to a treatment comprising both chemotherapy and radiotherapy treatment. The chemotherapy and radiotherapy can be concurrent or sequential. ICI other than PD-1 or PD-L1 ICI include cytotoxic T-lymphocyte antigen 4 (CTLA-4) ICI. Non-limiting examples of CTLA-4 ICI include Ipilimumab.

Non-limiting examples of kinase inhibitors include cytosolic tyrosine kinase (CTK) inhibitors (e.g. LFM-A13, piceatannol, idelalisib), Serine/Tyrosine kinase inhibitors (e.g. fasudil, sirolimus, temsirolimus, ribociclib), lipid kinase inhibitors (e.g. idelalisib), receptor tyrosine kinase (RTK) inhibitors (e.g. gefitinib, erlotinib, sunitinib, midostaurin).

In particular embodiments, the vaccine is a cancer vaccine. Non-limiting examples of cancer vaccines include antigen vaccines, whole cell vaccines, dendritic cell vaccines, DNA vaccines or anti-idiotype vaccines.

The term “vaccine” generally refers to a therapeutic or prophylactic pharmaceutical composition for in vivo administration to a subject, comprising a component to which a vaccinated subject is induced to raise an immune response, preferably a protective immune response, or immune tolerance (tolerising vaccines).

Optionally, the vaccine may further comprise one or more adjuvants for enhancing the immune response. Suitable adjuvants include, for example, but without limitation, saponin, mineral gels such as aluminium hydroxide, surface active substances such as lysolecithin, pluronic polyols, polyanions, peptides, oil or hydrocarbon emulsions, bacilli Calmette-Guerin (BCG), keyhole limpet hemocyanin (KLH), monophosphoryl lipid A (MPL), Corynebacterium parvum, oligodeoxynucleotides containing unmethylated CpG motif, and QS-21.

Optionally, the vaccine may further comprise one or more immunostimulatory molecules, or one or more molecules promoting immune tolerance. Non-limiting examples of such molecules include various cytokines, lymphokines and chemokines. By means of example, non-limiting examples of molecules with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc.

Tumour vaccines include vaccines that either a) prevent infections with cancer-causing viruses, b) treat existing cancer (therapeutic cancer vaccines) or c) prevent the development of cancer, or ameliorate its effects (prophylactic cancer vaccines).

One approach to produce tumour vaccines of type b) or c), also known as therapeutic or immunotherapeutic tumour vaccines, is to isolate tumour cells from a cancer patient, prepare an immunogenic composition from said tumour cells, for example, by rendering said tumour cells non-viable, preparing a lysate of said tumour cells, or isolating proteins from said tumour cells, and immunize a subject (e.g., the same cancer patient or another subject) with a vaccine comprising said immunogenic composition. The immunogenic composition contains tumour antigen(s) expressed by said tumour cells, whereby the vaccination can elicit or stimulate an immune response (e.g., B-cell or CTL response) against the tumour antigen(s) and the tumour cells expressing the tumour antigen(s). Another approach to therapeutic anti-cancer vaccination is to generate the immune response in situ in a patient. This enhances the anti-tumour immune response to tumour antigens released following lytic virus replication providing an in situ, patient specific anti-tumour vaccine as a result (examples of suitable oncolytic viruses include but are not limited to talimogene laherparepvec). Yet another approach is to immunize a patient with a compound that play a physiological role in cancer genesis, so that the human body eliminates said compound. In such case, the compound is a self-antigen or a self-hapten, i.e., it does not provoke a strong immune response when administered to the patient, but can elicit an adequate immune response when conjugated to a carrier.

Another approach to therapeutic anti-cancer vaccination includes dendritic cell vaccines. The term broadly encompasses vaccines comprising dendritic cells which are loaded with antigen(s) against which an immune reaction is desired.

The term “dendritic cell” (DC) may refer to any member of a diverse population of morphologically similar cell types found in lymphoid or non-lymphoid tissues. DC may include, for example, “professional” antigen presenting cells, and have a high capacity for sensitising MHC-restricted T cells. DCs may be recognised, for example, by function, by phenotype and/or by gene expression pattern, particularly by cell surface phenotype. These cells can be characterised by their distinctive morphology, high levels of surface MHC-class II expression and ability to present antigen to CD4+ and/or CD8+ T cells, particularly to naive T cells. Functionally, DCs may be identified by any suitable assay, known to one of skilled in the art, for determination of antigen presentation. Such assays may include, for example, testing the ability to stimulate antigen-primed and/or naive T cells by presentation of a test antigen, followed by determination of T cell proliferation, release of cytokines such as IL-2, and the like. Dendritic cells can be isolated or generated from a biological sample by methods well known in the art. Suitable biological samples for isolation or generation of DC include without limitation a peripheral blood sample, bone marrow sample, umbilical cord blood sample or the like. By means of an example but without limitation, DC present in a biological sample may be isolated by immunofluorescent or immunomagnetic labelling of select surface markers known to be expressed or not expressed by DC, coupled with a corresponding fluorescence activated cell sorting (FACS) gating strategy or immunomagnetic separation, respectively. Alternatively, DC can be generated from CD14+ monocytes by incubating them with suitable cytokines (Zhou & Tedder, Proc Natl Acad Sci USA. 1996, vol. 93, 2588-92).

The term “antigen loading” as used throughout this specification refers to a method or process of delivering one or more antigens to immune cells, such as particularly to antigen-presenting cells, such as more particularly to dendritic cells, such that the antigenic epitopes of the antigen(s) are presented on MHC, whether intracellular or on the immune cell surface. Typically, immune cells may be loaded with antigen(s) by a process comprising contacting or incubating the immune cells in vitro/ex vivo with a composition comprising the antigen(s) or a composition comprising nucleic acid(s) encoding the antigen(s) under conditions that permit the immune cells to contact, express (if needed), process and present the antigen(s) on MHC. The skilled person will know the incubation temperature and time periods sufficient to allow for effective loading of antigens. For example, incubation steps may be typically from between about 1 to about 2 or about 4 hours, at temperatures of between about 25° C. to about 37° C. and/or may be overnight at about 4° C., and the like. By means of an example, the immune cells may be contacted with a composition comprising an isolated antigen, for example, an antigen isolated from a naturally-occurring source of the antigen, or an antigen produced recombinantly by a suitable host or host cell expression system and isolated therefrom (e.g., a suitable bacterial, yeast, fungal, plant or animal host or host cell expression system), or produced recombinantly by cell-free transcription or translation, or non-biological nucleic acid or peptide synthesis. By means of another example, the immune cells may be contacted with a composition comprising a naturally-occurring source of the antigen, i.e., substantially without isolating the antigen from said naturally-occurring source. For instance, the immune cells may be contacted with a composition comprising cells which naturally express the antigen or cell debris of such cells, e.g., tumour cells expressing tumour antigen(s). Suitably, such cells may be rendered non-viable and preferably lysed, for example, killed and preferably lysed by a mechanical, chemical or physical treatment, such as heat killed, apoptotic, necrotic or otherwise processed. By means of a further example, the immune cells may be contacted with cells of a suitable host or host cell expression system which recombinantly produce the antigen, i.e., substantially without isolating the antigen from said cells. Suitably, such cells may be rendered non-viable and preferably lysed, for example, killed and preferably lysed by a mechanical, chemical or physical treatment, such as heat killed or otherwise processed. Immune cells may also be loaded with an antigen by introducing into the immune cells a nucleic acid, commonly a recombinant nucleic acid, encoding the antigen, whereby the immune cells express the antigen.

The term “antibody-drug conjugate” or “ADC” as used herein refers to an immunoconjugate comprising a monoclonal antibody fused to a cytotoxic drug, optionally via a linker, which is able to kill neoplastic cells while sparing healthy cells. Non-limiting example include gemtuzumab ozagamicin, brentuximab vedotin, trastuzumab emtansine, inotuzumab ozogamicin, polatuzumab vedotin-piiq, enfortumab vedotin, trastuzumab deruxtecan or sacituzumab govetecan.

Another a further embodiment, the kinase substrates carrying phosphorylation sites according to the present invention are located or immobilized on a solid support, and preferably a porous solid support. Preferably said immobilized kinase substrates carrying phosphorylation sites will be immobilized proteins, peptides or peptide mimetics. More preferably, the peptides are immobilized on a solid support.

As used herein “peptide mimetics” refers to organic compounds which are structurally similar to peptides and similar to the peptide sequences list in Table 2 or Table 3. The peptide mimetics are typically designed from existing peptides to alter the molecules characteristics. Improved characteristics can involve, for example improved stability such as resistance to enzymatic degradation, or enhanced biological activity, improved affinity by restricted preferred conformations and ease of synthesis. Structural modifications in the peptidomimetic in comparison to a peptide, can involve backbone modifications as well as side chain modification.

Depending on the type of kinase activity measurement method the solid support on which the proteins, peptides or peptide mimetics are fixed may vary. Whereas in ELISA the protein kinase substrates are attached to the surface of the microtiterplates, in microarrays the protein kinase substrates are immobilized on and/or in the microarray substrate. Alternatively the substrates are synthesized in-situ direct on the microarray substrate.

In a preferred embodiment of the present invention the protein kinase substrates are immobilized on an array, and preferably a microarray, of protein kinase substrates wherein the protein kinase substrates are immobilized onto a solid support or another carrier. The immobilization can be either the attachment or adherence of two or more protein kinase substrate molecules to the surface of the carrier including attachment or adherence to the inner surface of said carrier in the case of e.g. a porous or flow-through solid support.

In a preferred embodiment of the present invention, the array of protein kinase substrates is a flow-through array. The flow-through array as used herein could be made of any carrier material having oriented through-going channels as are generally known in the art, such as for example described in PCT patent publication WO 01/19517. Typically the carrier is made from a metal oxide, glass, silicon oxide or cellulose. In a particular embodiment the carrier material is made of a metal oxide selected from the group consisting of zinc oxide, zirconium oxide, tin oxide, aluminium oxide, titanium oxide and thallium; in a more particular embodiment the metal oxide consists of aluminium oxide.

Accordingly, in a further embodiment of the present invention said array is a Pamchip®, preferably a PTK Pamchip®, preferably the PTK Pamchip® (PamGene, 's-Hertogenbosch, The Netherlands)

In a further embodiment, the present invention relates to a method according to the present invention wherein said solid support (microarray) comprises all 142 of the peptide markers as listed in Table 2 or all 62 of the peptide markers as listed in Table 3 immobilized thereto.

Phosphorylation levels can also be measured according to the invention, without the necessity to generate phosphorylation profiles thereof. Also for this embodiment, the amount and the type of peptides, proteins or peptide mimetics to be used is as described above.

Another embodiment of the present invention regards the use of the method according to the present invention for assessing susceptibility of a patient in need of an ICI to a PD-1 or PD-L1 ICI.

Another embodiment of the present invention regards the use of the method according to the present invention for assessing the pharmaceutical value of a PD-1 or PD-L1 ICI.

Another embodiment of the present invention regards the use of the method according to the present invention for assessing the clinical value of a PD-1 or PD-L1 ICI.

As used herein when assessing susceptibility to a drug, the pharmaceutical value of a drug or the clinical value of a drug, this comprises the assessment of the resistance of a subject or patient to said PD-1 or PD-L1 ICI.

Also provided herein is a computer program product for use in conjunction with a computer having a processor and a memory connected to the processor, said computer program product comprising a computer readable storage medium having a computer program mechanism encoded thereon, wherein said computer program mechanism may be loaded into the memory of said computer and cause said computer to carry out the method according to the present invention.

Also provided herein is a computer system comprising a processor, and a memory coupled to said processor and encoding one or more programs, wherein said one or more programs instruct the processor to carry out the methods according to the present invention.

The finding of present inventors that the kinase activity of at least two kinases independently selected from within at least two families of kinases selected from the group consisting of the VEGFR or PDGFR family of kinases; the SRC family of kinases; the SYK family of kinases; the TAM family of kinases; the JakA family of kinases; and the ALK family of kinases, preferably selected from the group consisting the VEGFR or PDGFR family of kinases; the SRC family of kinases; the SYK family of kinases; and the TAM family of kinases, can be used as a marker for predicting the response to treatment with a PD-1 or PD-L1 ICI in a patient in need of an ICI can also be converted into a kit which allows making that prediction of response based on the kinase activity of at least two kinases independently selected from within at least two families of kinases selected from the group consisting of the VEGFR or PDGFR family of kinases; the SRC family of kinases; the SYK family of kinases; the TAM family of kinases; the JakA family of kinases; and the ALK family of kinases, preferably selected from the group consisting the VEGFR or PDGFR family of kinases; the SRC family of kinases; the SYK family of kinases; and the TAM family of kinases, and which can assist a surgeon, researcher, veterinarian, medical doctor or other clinician, in his decision making process on whether to administer a certain PD-1 or PD-L1 ICI to a patient in need of an ICI or not.

Hence, an aspect of the invention provides a kit for predicting the response of a patient in need of an ICI to a PD-1 or PD-L1 ICI, comprising means for measuring the kinase activity of at least 2, at least 3, at least 4, at least 5, at least 6, at least 7, at least 8, at least 9, at least 10, or at least 11, at least 12, at least 13, at least 14, at least 15, at least 16, at least 17, at least 18, at least 19, at least 20, at least 21, at least 22, at least 23, at least 24, or at least 25, at least 26, at least 27 or at least 28, preferably at least 14, kinases independently selected from within at least two families of kinases selected from the group consisting of:
the VEGFR or PDGFR family of kinases, preferably the VEGFR or PDGFR family of kinases consisting of VEGFR1, VEGFR2, VEGFR3, PDGFRalpha, PDGFRbeta, CSF-1R, Kit, FLT3, and any combination thereof;
the SRC family of kinases, preferably the SRC family of kinases consisting of SRC, YES, FYN, FGR, LCK, HCK, BLK, LYN, FRK, and any combination thereof;
the SYK family of kinases, preferably the SYK family of kinases consisting of SYK, ZAP-70, and any combination thereof;
the TAM family of kinases, preferably the TAM family of kinases consisting of TYRO3, AXL, MERTK, and any combination thereof;
the JakA family of kinases, preferably the JAK family of kinases consisting of JAK1, JAK2, JAK3, and TYK2 and any combination thereof; and
the ALK family of kinases, preferably the ALK/LTK family of kinases consisting of ALK, LTK, and any combination thereof,
   and optionally at least one, preferably at least two, kinases of the group consisting of TRKC, RON, and any combination thereof;
   preferably means for measuring the kinase activity of at least 2, at least 3, at least 4, at least 5, at least 6, at least 7, at least 8, at least 9, at least 10, or at least 11, at least 12, at least 13, at least 14, at least 15, at least 16, at least 17, at least 18, at least 19, at least 20, at least 21, or at least 22, kinases independently selected from within at least two families of kinases selected from the group consisting of:
the VEGFR or PDGFR family of kinases, preferably the VEGFR or PDGFR family of kinases consisting of VEGFR1, VEGFR2, VEGFR3, PDGFRalpha, PDGFRbeta, CSF-1R, Kit, FLT3, and any combination thereof;
the SRC family of kinases, preferably the SRC family of kinases consisting of SRC, YES, FYN, FGR, LCK, HCK, BLK, LYN, FRK, and any combination thereof;
the SYK family of kinases, preferably the SYK family of kinases consisting of SYK, ZAP-70, and any combination thereof; and
the TAM family of kinases, preferably the TAM family of kinases consisting of TYRO3, AXL, MERTK, and any combination thereof;
   and optionally at least one, at least two, or at least three, preferably at least three, kinases of the group consisting of TRKC, RON, ALK and any combination thereof;
   in a blood sample obtained from said patient in need of an ICI.

The terms “kit of parts” and “kit” as used throughout this specification refer to a product containing components necessary for carrying out the specified methods, packed so as to allow their transport and storage. Materials suitable for packing the components comprised in a kit include crystal, plastic (e.g., polyethylene, polypropylene, polycarbonate), bottles, flasks, vials, ampules, paper, envelopes, or other types of containers, carriers or supports. Where a kit comprises a plurality of components, at least a subset of the components (e.g., two or more of the plurality of components) or all of the components may be physically separated, e.g., comprised in or on separate containers, carriers or supports. The components comprised in a kit may be sufficient or may not be sufficient for carrying out the specified methods, such that external reagents or substances may not be necessary or may be necessary for performing the methods, respectively. Typically, kits are employed in conjunction with standard laboratory equipment, such as liquid handling equipment, environment (e.g., temperature) controlling equipment, analytical instruments, etc. The present kits may also include some or all of solvents, buffers, enzymes, detectable labels, detection reagents, and control formulations (positive and/or negative), useful in the specified methods. Typically, the kits may also include instructions for use thereof, such as on a printed insert or on a computer readable medium. The terms may be used interchangeably with the term “article of manufacture”, which broadly encompasses any man-made tangible structural product, when used in the present context.

In particular embodiments, said kit comprises at least one reference kinase activity profile being representative for a good or poor responder to said PD-1 or PD-L1 ICI. In particular embodiments, said kit comprises a first and a second reference kinase activity profile; said first reference kinase activity profile being representative for a good responder to said PD-1 or PD-L1 ICI and said second reference kinase activity profile being representative for a poor responder to said PD-1 or PD-L1 ICI.

In particular embodiments, said kit comprises at least one reference sample obtained from one or more subjects known to be a good or poor responder to said PD-1 or PD-L1 ICI. In particular embodiments, said kit comprises at reference sample obtained from one or more subjects known to be a good responder and a reference sample obtained from one or more subjects known to be a poor responder to said PD-1 or PD-L1 ICI.

In particular embodiments, said kit comprises a classifier, as described elsewhere herein.

In particular embodiments, said kit comprises an IAC sample, as described elsewhere herein.

In particular embodiments, said kit comprises a computer readable storage medium having recorded thereon one or more programs for carrying out the method for predicting the response of a patient in need of an ICI to a PD-1 or PD-L1 ICI as taught herein.

In particular embodiments, the kit comprises means for measuring the kinase activity of at least 2, at least 3, at least 4, at least 5, at least 6, at least 7, at least 8, at least 9, at least 10, at least 11, at least 12, at least 13, or all 14 kinases selected from the group consisting of VEGFR1, VEGFR3, FLT3, SRC, FYN, BLK, SYK, ZAP70, TYRO-3, AXL, MER, TRKC, RON, and ALK.

In particular embodiments, the kit comprises means for measuring the kinase activity of all of VEGFR1, VEGFR3, FLT3, SRC, FYN, BLK, SYK, ZAP70, TYRO-3, AXL, MER, TRKC, RON, and ALK.

In particular embodiments, the kit comprises means for measuring the kinase activity of at least 2, at least 3, at least 4, at least 5, at least 6, at least 7, at least 8, at least 9, at least 10, at least 11, at least 12, at least 13, at least 14, at least 15 or all 16 kinases selected from the group consisting of VEGFR1, VEGFR3, FLT3, SRC, FYN, BLK, SYK, ZAP70, TYRO-3, AXL, MER, TRKC, RON, ALK, LTK, and JAK1.

In particular embodiments, the kit comprises means for measuring the kinase activity of all of VEGFR1, VEGFR3, FLT3, SRC, FYN, BLK, SYK, ZAP70, TYRO-3, AXL, MER, TRKC, RON, ALK, LTK, and JAK1.

The means for measuring the kinase activity of kinases of the VEGFR or PDGFR family of kinases; the SRC family of kinases; the SYK family of kinases; the TAM family of kinases; the JakA family of kinases; the ALK family of kinases, and optionally at least one kinase of the group of kinases consisting of TRKC and RON can be any means known in the art to determine kinase activity, such as an array comprising peptide markers.

In particular embodiments, the means for measuring the kinase activity of

at least 2, at least 3, at least 4, at least 5, at least 6, at least 7, at least 8, at least 9, at least 10, or at least 11, at least 12, at least 13, at least 14, at least 15, at least 16, at least 17, at least 18, at least 19, at least 20, at least 21, at least 22, at least 23, at least 24, at least 25, at least 26, at least 27 or at least 28, preferably at least 16, kinases independently selected from within at least two, at least three, at least four, at least five, or at least six, families of kinases selected from the group consisting of:
the VEGFR or PDGFR family of kinases, preferably the VEGFR or PDGFR family of kinases consisting of VEGFR1, VEGFR2, VEGFR3, PDGFRalpha, PDGFRbeta, CSF-1R, Kit, FLT3, and any combination thereof;
the SRC family of kinases, preferably the SRC family of kinases consisting of SRC, YES, FYN, FGR, LCK, HCK, BLK, LYN, FRK, and any combination thereof;
the SYK family of kinases, preferably the SYK family of kinases consisting of SYK, ZAP-70, and any combination thereof;
the TAM family of kinases, preferably the TAM family of kinases consisting of TYRO3, AXL, MERTK, and any combination thereof;
the JakA family of kinases, preferably the JakA family of kinases consisting of JAK1, JAK2, JAK3, and TYK2, and any combination thereof; and
the ALK family of kinases, preferably the ALK family of kinases consisting of ALK, LTK, and any combination thereof,
   and optionally at least one, preferably at least two, kinases of the group consisting of TRKC, RON, and any combination thereof;
   preferably means for measuring the kinase activity of at least 2, at least 3, at least 4, at least 5, at least 6, at least 7, at least 8, at least 9, at least 10, or at least 11, at least 12, at least 13, at least 14, at least 15, at least 16, at least 17, at least 18, at least 19, at least 20, at least 21, or at least 22, preferably at least 11, kinases independently selected from within at least two, at least three, or at least four, families of kinases selected from the group consisting of:
the VEGFR or PDGFR family of kinases, preferably the VEGFR or PDGFR family of kinases consisting of VEGFR1, VEGFR2, VEGFR3, PDGFRalpha, PDGFRbeta, CSF-1R, Kit, FLT3, and any combination thereof;
the SRC family of kinases, preferably the SRC family of kinases consisting of SRC, YES, FYN, FGR, LCK, HCK, BLK, LYN, FRK, and any combination thereof;
the SYK family of kinases, preferably the SYK family of kinases consisting of SYK, ZAP-70, and any combination thereof; and
the TAM family of kinases, preferably the TAM family of kinases consisting of TYRO3, AXL, MERTK, and any combination thereof;
   and optionally at least one, at least two, or at least three, kinases of the group consisting of TRKC, RON, ALK and any combination thereof;
   in a blood sample obtained from said patient in need of an ICI,
   is at least one array comprising all of the 142 peptide markers as listed in Table 2 or all of the 62 peptide markers as listed in Table 3.

Also provided herein is the use of the kinase activity of at least two kinases independently selected from within at least two families of kinases selected from the group consisting of: the VEGFR or PDGFR family of kinases; the SRC family of kinases; the SYK family of kinases; the TAM family of kinases; the JakA family of kinases; and the ALK family of kinases;

in a blood sample obtained from a patient in need of an ICI for predicting the response of said patient to a PD-1 or PD-L1 ICI.

In a particular embodiment, the present invention relates to the use of the kinase activity of at least 2, at least 3, at least 4, at least 5, at least 6, at least 7, at least 8, at least 9, at least 10, at least 11, at least 12, at least 13, or all 14 kinases selected from the group consisting of VEGFR1, VEGFR3, FLT3, SRC, FYN, BLK, SYK, ZAP70, TYRO-3, AXL, MER, TRKC, RON, ALK, LTK, and JAK, as listed in Table 1, for predicting the response of a patient in need of an ICI to a PD-1 or PD-L1 ICI.

Since the present inventors have identified a surprisingly useful set of kinases to be used in methods for determining the prediction of response to a PD-1 or PD-L1 ICI, of a patient in need of an ICI, the person skilled in the art may carry out any method as defined above wherein he measures the kinase activity of

at least 2, at least 3, at least 4, at least 5, at least 6, at least 7, at least 8, at least 9, at least 10, or at least 11, at least 12, at least 13, at least 14, at least 15, at least 16, at least 17, at least 18, at least 19, at least 20, at least 21, at least 22, at least 23, at least 24, at least 25, at least 26, at least 27 or at least 28, preferably at least 14, kinases independently selected from within at least 2, at least 3, at least 4, at least 5 or at least 6, preferably at least 4, families of kinases selected from the group consisting of:
the VEGFR or PDGFR family of kinases, preferably the VEGFR or PDGFR family of kinases consisting of VEGFR1, VEGFR2, VEGFR3, PDGFRalpha, PDGFRbeta, CSF-1R, Kit, FLT3, and any combination thereof;
the SRC family of kinases, preferably the SRC family of kinases consisting of SRC, YES, FYN, FGR, LCK, HCK, BLK, LYN, FRK, and any combination thereof;
the SYK family of kinases, preferably the SYK family of kinases consisting of SYK, ZAP-70, and any combination thereof;
the TAM family of kinases, preferably the TAM family of kinases consisting of TYRO3, AXL, MERTK, and any combination thereof;
the JakA family of kinases, preferably the JAK family of kinases consisting of JAK1, JAK2, JAK3, and TYK2 any combination thereof; and
the ALK family of kinases, preferably the ALK family of kinases consisting of ALK, LTK, and any combination thereof,
   and optionally at least one or at least two kinases of the group consisting of TRKC, RON, and any combination thereof;
   preferably means for measuring the kinase activity of at least 2, at least 3, at least 4, at least 5, at least 6, at least 7, at least 8, at least 9, at least 10, or at least 11, at least 12, at least 13, at least 14, at least 15, at least 16, at least 17, at least 18, at least 19, at least 20, at least 21, or at least 22, preferably at least 11, kinases independently selected from within at least two families of kinases selected from the group consisting of:
the VEGFR or PDGFR family of kinases, preferably the VEGFR or PDGFR family of kinases consisting of VEGFR1, VEGFR2, VEGFR3, PDGFRalpha, PDGFRbeta, CSF-1R, Kit, FLT3, and any combination thereof;
the SRC family of kinases, preferably the SRC family of kinases consisting of SRC, YES, FYN, FGR, LCK, HCK, BLK, LYN, FRK, and any combination thereof;
the SYK family of kinases, preferably the SYK family of kinases consisting of SYK, ZAP-70, and any combination thereof; and
the TAM family of kinases, preferably the TAM family of kinases consisting of TYRO3, AXL, MERTK, and any combination thereof;
   and optionally at least one, at least two, or at least three, kinases of the group consisting of TRKC, RON, ALK and any combination thereof.

Also this method may be carried out using the amount and type of peptides, proteins or protein mimetics as defined above. The formats for carrying out these methods are also as for the methods described above.

Also provided herein is a method of treating a patient in need of an ICI (i.e. in need of treatment with an ICI), comprising

(I) determining the response of a patient in need of an ICI, to treatment with a PD-1 or PD-L1 ICI comprising the steps of
(a) measuring the kinase activity of at least two kinases independently selected from within at least two families of kinases selected from the group consisting of the VEGFR or PDGFR family of kinases; the SRC family of kinases; the SYK family of kinases; the TAM family of kinases; the JakA family of kinases; and the ALK family of kinases, preferably selected from the group consisting of: the VEGFR or PDGFR family of kinases; the SRC family of kinases; the SYK family of kinases; and the TAM family of kinases, in a blood sample obtained from said patient, thereby providing a kinase activity profile of said blood sample; and
(b) determining from said kinase activity profile the response of said patient to treatment with said PD-1 or PD-L1 ICI; and
   (II) treating said patient with said PD-1 or PD-L1 ICI if said patient is determined to be responsive to said PD-1 or PD-L1 ICI; or treating said patient with an antineoplastic therapy other than said PD-1 or PD-L1 ICI if said patient is determined to be unresponsive to said PD-1 or PD-L1 ICI.

In other words, the PD-1 or PD-L1 ICI may be used in the treatment of a neoplastic disease, preferably a neoplastic disease selected from the group consisting of acute myeloid leukaemia, advanced cancer, anal basaloid carcinoma, basaloid squamous cell carcinoma, biliary tract carcinoma, bladder cancer, brain cancer, breast cancer, cervical cancer, clear-cell renal cell carcinoma, colorectal cancer, diffuse large b-cell lymphoma, endometrial cancer, epithelial ovarian cancer, oesophageal cancer, gastric cancer, glioblastoma, head and neck cancer, hepatocellular carcinoma, Hodgkin lymphoma, kidney cancer, liver cancer, lung cancer, malignant melanoma, melanoma, merkel cell carcinoma, mesothelioma, multiple myeloma, non-small cell lung carcinoma, oropharyngeal carcinoma, ovarian cancer, pancreatic cancer, prostate cancer, small cell lung cancer, triple negative breast cancer, and urothelial cancer, comprising:

(I) determining the response of a patient in need of an ICI, to treatment with a PD-1 or PD-L1 ICI comprising the steps of
(a) measuring the kinase activity of at least two kinases independently selected from within at least two families of kinases selected from the group consisting of: the VEGFR or PDGFR family of kinases; the SRC family of kinases; the SYK family of kinases; the TAM family of kinases; the JakA family of kinases; and the ALK family of kinases, preferably selected from the group consisting of: the VEGFR or PDGFR family of kinases; the SRC family of kinases; the SYK family of kinases; and the TAM family of kinases, in a blood sample obtained from said patient, thereby providing a kinase activity profile of said blood sample; and
(b) determining from said kinase activity profile the response of said patient to treatment with said PD-1 or PD-L1 ICI; and
   (II) administering said PD-1 or PD-L1 ICI to said patient if said patient is determined to be responsive to said PD-1 or PD-L1 ICI or not administering said PD-1 or PD-L1 ICI to said patient if said patient is determined to be unresponsive to said PD-1 or PD-L1 ICI.

The terms “treat” or “treatment” encompass both the therapeutic treatment of an already developed disease or condition, such as the therapy of an already developed neoplastic disease, as well as prophylactic or preventive measures, wherein the aim is to prevent or lessen the chances of incidence of an undesired affliction, such as to prevent occurrence, development and progression of a neoplastic disease. Beneficial or desired clinical results may include, without limitation, alleviation of one or more symptoms or one or more biological markers, diminishment of extent of disease, stabilized (i.e., not worsening) state of disease, delay or slowing of disease progression, amelioration or palliation of the disease state, and the like. “Treatment” can also mean prolonging survival as compared to expected survival if not receiving treatment.

The PD-1 or PD-L1 ICI for use and methods as taught herein allow to administer a therapeutically effective amount of a PD-1 or PD-L1 ICI, in patients in need of an ICI who will benefit from such treatment. The term “therapeutically effective amount” as used herein, refers to an amount of active compound or pharmaceutical agent that elicits the biological or medicinal response in a subject that is being sought by a surgeon, researcher, veterinarian, medical doctor or other clinician, which may include inter alia alleviation of the symptoms of the disease or condition being treated. Methods are known in the art for determining therapeutically effective doses of a PD-1 or PD-L1 ICI as taught herein. In certain embodiments, said PD-1 or PD-L1 ICI is formulated into and administered as pharmaceutical formulations or compositions. Such pharmaceutical formulations or compositions may be comprised in a kit of parts.

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, antioxidants, tonicity controlling agents, absorption delaying agents, and the like. The use of such media and agents for pharmaceutical active substances is well known in the art. Except insofar as any conventional media or agent is incompatible with the active substance, its use in the therapeutic compositions may be contemplated.

Illustrative, non-limiting carriers for use in formulating the pharmaceutical compositions include, for example, oil-in-water or water-in-oil emulsions, aqueous compositions with or without inclusion of organic co-solvents suitable for intravenous (IV) use, liposomes or surfactant-containing vesicles, microspheres, microbeads and microsomes, powders, tablets, capsules, suppositories, aqueous suspensions, aerosols, and other carriers apparent to one of ordinary skill in the art.

Pharmaceutical compositions as intended herein may be formulated for essentially any route of administration, such as without limitation, oral administration (such as, e.g., oral ingestion or inhalation), intranasal administration (such as, e.g., intranasal inhalation or intranasal mucosal application), parenteral administration (such as, e.g., subcutaneous, intravenous, intramuscular, intraperitoneal or intrasternal injection or infusion), transdermal or transmucosal (such as, e.g., oral, sublingual, intranasal) administration, topical administration, rectal, vaginal or intra-tracheal instillation, and the like. In this way, the therapeutic effects attainable by the methods and compositions can be, for example, systemic, local, tissue-specific, etc., depending of the specific needs of a given application.

The dosage or amount of the present PD-1 or PD-L1 ICI used, optionally in combination with one or more other active compounds to be administered, depends on the individual case and is, as is customary, to be adapted to the individual circumstances to achieve an optimum effect. Thus, it depends on the nature and the severity of the disorder to be treated, and also on the sex, age, body weight, general health, diet, mode and time of administration, and individual responsiveness of the patient to be treated, on the route of administration, efficacy, metabolic stability and duration of action of the compounds used, on whether the therapy is acute or chronic or prophylactic, or on whether other active compounds are administered in addition to the PD-1 or PD-L1 ICI as described herein.

Without limitation, depending on the type and severity of the disease, a typical dosage of a PD-1 or PD-L1 ICI as disclosed herein, or combinations of two or more such PD-1 or PD-L1 ICIs, might range from about 1 ag/kg to 1 g/kg of body weight or more, depending on the factors mentioned above. For instance, at an ICI treatment interval of 2 to 4 weeks a dosage of the agent(s) may range from about 0.5 mg/kg to 50 mg/kg of body weight or about 100-2000 mg per patient per treatment cycle. For repeated administrations over several weeks or months or longer, depending on the condition, the treatment is sustained until a desired suppression of disease symptoms occurs.

In certain embodiments, the a PD-1 or PD-L1 ICI may be administered at least once a month during the treatment, for example the PD-1 or PD-L1 ICI may be administered at least once every three weeks during the treatment, for example the a PD-1 or PD-L1 ICI may be administered at least once every two weeks during the treatment.

In certain embodiments, the PD-1 or PD-L1 ICI may be administered daily during the treatment

In certain embodiments, the PD-1 or PD-L1 ICI or pharmaceutical formulation as taught herein may be used alone or in combination with one or more active compounds that are suitable in the treatment of neoplastic diseases (i.e., combination therapy). The latter can be administered before, after, or simultaneously with the administration of the PD-1 or PD-L1 ICI or pharmaceutical formulation as taught herein.

The person skilled in the art will understand that the different embodiments of the methods for determining the response of a patient in need of an ICI to treatment with a PD-1 or PD-L1 ICI are applicable to all methods (e.g. methods of treatment), uses, kits, computer program products and computer systems as described herein, and vice versa.

The present invention is hereafter exemplified by the illustration of particular, non-limiting examples.

### EXAMPLES

### Example 1. Kinase Activity Profile Allows Predicting the Response of a Patient in Need of an ICI to an Anti-CTLA4 or PD-1 or PD-L1 ICI

1. Materials and Methods

Patient Population and Study Workflow

Patients with irresectable stage Ill/IV advanced melanoma or stage IV NSCLC were included if they had received intravenous monotherapy with either ipilimumab (anti-CTLA4; 3 mg/kg every 3 weeks for 4 courses), nivolumab (anti-PD-1; 3 mg/kg every 2 weeks until a maximum of 2 years) or pembrolizumab (anti-PD1; 2 mg/kg every 3 weeks until a maximum of 2 years) as standard of care at the Erasmus University Medical Center (Rotterdam, The Netherlands), Leiden University Medical Center (Leiden, The Netherlands) and University Hospital Zurich (Zurich, Switzerland), all being referral hospitals. Patients who received ICI combination therapy or who were treated with a prior line of any form of immunotherapy were excluded, pretreatment of corticosteroids was not considered. The study was approved by the independent ethics committee board (reference numbers: MEC 16-011, P11-016, and MEC 14-0425, respectively) and in accordance with the revised WMA Declaration of Helsinki on human rights. Blood samples were collected at baseline after written informed consent of the patients.

Data Collection

Binary grouping was performed into patients with (responders) or without (non-responders) clinical benefit. Clinical benefit was based on response evaluation criteria in solid tumors (RECIST) version 1.1. For determination of best overall response (BOR), confirmation of complete or partial response (CR/PR) was not required but a minimum duration of 90 days was required for stable disease (SD). Patients with a CR, PR or SD as BOR were considered to have obtained clinical benefit after ICIs and were defined as responders. Patients with progressive disease (PD) were defined as non-responders. Additionally, binary grouping of all patients based on the progression-free survival (PFS), measured from start of treatment to death or the first evaluation time point that PD is detected, was based on late (>140 days) or no progression (responders) versus patients with early progression within 140 days (non-responders). Clinical parameters and chemistry or blood count parameters were evaluated at baseline and included age, gender, WHO performance score, pathological tumor type, presence of brain metastases, neutrophil to lymphocyte ratio (NLR) and serum lactate dehydrogenase concentration (LDH).

Preparation of PBMC Lysate

Venous blood of patients was collected using either sodium-heparin or EDTA as anticoagulant, and isolation of PBMCs was done within 4 hours or within 24 hours depending on the local study protocol (Table 4).

TABLE 4
Local study protocols

| | | | | PBMC isolation | |
|---|---|---|---|---|---|
| Cohort | ICI | Study site | Anticoagulant | within | Erythrolysis |
| Mel-CTLA4-A | CTLA-4 | Center A | Na-Hep | 4 h | no |
| Mel-CTLA4-B | CTLA-4 | Center B | EDTA | 24 h | yes |
| Mel-PD1-A | PD-1 | Center A | Na-Hep | 4 h | no |
| Mel-PD1-B | PD-1 | Center C | EDTA | 24 h | no |
| NSCLC-PD1 | PD-1 | Center C | EDTA | 24 h | no |

Table 4: Five patient cohorts were evaluated in this study. The cohorts are based on the malignancy (melanoma or NSCLC patients), the type of ICI therapy administered and the center were the samples were collected. Patient number per cohort and anticoagulant used for blood collection is given. An erythrocyte lysis step was performed during PBMC isolation for cohort Mel-CTLA4-B.

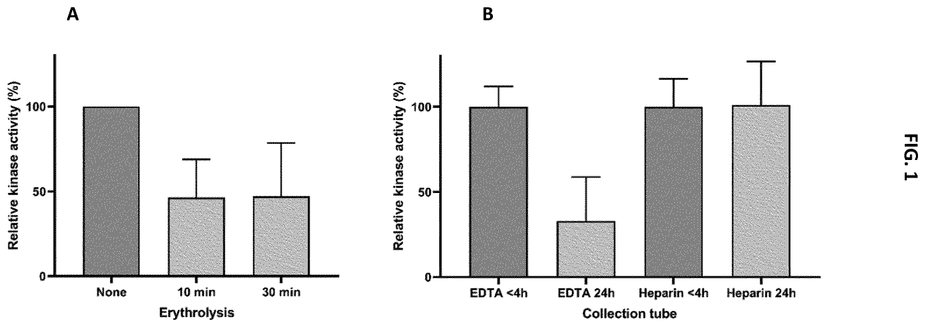

PBMC were isolated by density gradient centrifugation and cryopreserved until further use. Erythrocyte lysis was only performed in the Mel-CTLA4-B cohort if PBMC still contained considerable erythrocyte contamination after isolation. Cryopreserved cells were thawed and washed with phosphate buffered saline and cells were lysed for kinase activity analysis. Importantly, kinase activity was affected by erythrocyte lysis during PBMC isolation (FIG. 1a). Moreover, overall reduced kinase activity was observed in PBMCs that were isolated only after 24 hours from blood collected in EDTA anticoagulated tubes (FIG. 1b).

Kinase Activity Profiling

Kinase activity profiles of the lysates were measured using PTK PamChip-96 microarrays (PamGene, 's-Hertogenbosch, The Netherlands). Incubation and read-out of the microarrays was performed with 96 arrays in parallel on a PamStation-96. Typically, 3-4 technical replicates of each sample were measured in the same run. The time course of peptide phosphorylation was followed by recording fluorescent images of the microarrays during the incubation. The fluorescence images recorded by the PamStation-96 were quantified by automated image analysis in Bionavigator 6.3.67 (PamGene, 's-Hertogenbosch, The Netherlands). Further analysis was performed using signals after local background subtraction in Bionavigator 6.3.67 interfaced to the open source statistical program R 3.3.1 (R-project, www.rproject.org). Further analysis was performed per cohort using an analysis pipeline consisting of a quality check, removal of outlier replicates, data transformation and averaging of the kinase signal replicates (see section “Quality check and data processing of peptide signals” below for details). Principal Component Analysis (PCA) was performed on the transformed and filtered data and PCA scores were inspected to identify systematic variation, e.g. as a result of different sub-cohorts and/or the use of different PamStation-96 microarray plates. Systematic variation was handled by applying ComBat (Johnson, W. E., Li, C. & Rabinovic, A. Adjusting batch effects in microarray expression data using empirical Bayes methods. Biostatistics 8, 118-127 (2007); Zhang, Y., Jenkins, D. F., Manimaran, S. & Johnson, W. E. Alternative empirical Bayes models for adjusting for batch effects in genomic studies. BMC Bioinformatics 19, 262 (2018)) batch correction, outlier patient samples were removed.

Quality Check and Data Processing of Peptide Signals

The first step was an inspection on the technical quality of the data in which a low portion of the arrays showing clear visual defects (e.g. broken membrane, large stains) or technical replicates clearly deviating from the other replicates of the same samples were removed from the data. The data is then log-transformed (CTLA-4 cohorts: Mel-CTLA4-A, Mel-CTLA4-B) or normalized using the VSN method (W. Huber, A. von Heydebreck, H. Sultmann, A. Poustka, and M. Vingron, “Variance stabilization applied to microarray data calibration and to the quantification of differential expression.,” Bioinformatics, vol. 18 Suppl 1, pp. 596-104, 2002) (PD-1 cohorts: Mel-PD1-A, Mel-PD1-B, NSCLC-PD1). For the log-transformation a small fraction of negative values in the (background corrected) signals was handled by setting all signals <1 or equal to 1 before log-transformation. The presence of a signal on the kinase peptide substrates was determined based on the detection of a positive trend in the recorded phosphorylation time course. Peptides for which such a trend could not be detected in >75% of the samples were filtered out before further analysis. Effectively, this results in the removal of a set of peptides (31-56) with absent or very low signal throughout the experiment, leaving 88-113 peptides for further analysis. Next, technical replicates of each patient sample were averaged resulting in a single kinase activity profile per included patient for subsequent analysis.

Systematic effects between sub-cohorts and measurement batches were apparent from PCA analysis. For the cohorts with patients who were treated with PD-1 ICIs this was handled by applying a ComBat batch correction (Johnson, W. E., Li, C. & Rabinovic, A. Adjusting batch effects in microarray expression data using empirical Bayes methods. Biostatistics 8, 118-127 (2007); Zhang, Y., Jenkins, D. F., Manimaran, S. & Johnson, W. E. Alternative empirical Bayes models for adjusting for batch effects in genomic studies. BMC Bioinformatics 19, 262 (2018)) using the PamStation96 microarray plate identifier as the batch variable. For the small Mel-CTLA4-A cohort no such correction was necessary. For the Mel-CTLA4-B cohort a batch effect was observed and corrected for the batches in which the samples were lysed. A small number of outlier patient samples observed in the PCA scores or heat map visualizations of the transformed profiles were removed. In most cases these were samples showing low overall kinase activity compared to the rest of the samples. A number of 1, 2, and 3 of these low-signal samples were removed for the Mel-CTLA4-B, Mel-PD1-B, and Mel-PD1-A cohorts, respectively. For the NSCLC-PD1 cohort a total of 8 outlier samples needed to be removed, both with high and low overall activity compared to the rest of the samples. Reasons for removing measured patient samples from the analysis was unavailability of clinical outcome data or if, after reevaluation, patients were observed not to fit the selection criteria described above, e.g. because they received an ICI combination treatment.

Statistical Analysis and Bioinformatics

Evaluable kinase activity profiles were correlated to the response to ICIs. Univariate (per peptide) analysis was performed using a two-sided two sample t-test with binary grouping as a covariate (responders versus non-responders). Peptides with p<0.05 were regarded as significant, in addition, the proportion of false discoveries in a set of significant peptides was estimated using the FDR method of Benjamini & Hochberg. Classification analysis was performed using Partial Least Squares Discriminant Analysis (PLS-DA) with the patients divided in binary groups as previously described (Arni, S., et al. Ex vivo multiplex profiling of protein tyrosine kinase activities in early stages of human lung adenocarcinoma. Oncotarget 8, 68599-68613 (2017)). In short, a classification model is trained using all peptides included during the pre-processing step, i.e. without prior peptide selection based on responder and non-responder differences. The resulting model is a set of coefficients (one for each peptide+an offset) that can be applied to new observations to obtain a score that predicts the classification in either of both groups. For each cohort, the Correct Classification Rate (CCR) was estimated using cross validation. Approximate 90% binomial confidence intervals (CI₉₀) for the CCR estimates were obtained using the exact method (note that a CI₉₀ implies 95% confidence that the CCR is higher than the lower confidence limit). A prediction of the kinases responsible for the changes in peptide phosphorylation in the kinomic profiles were obtained using the Upstream Kinase Analysis tool in Bionavigator 6.3.67 (PamGene, 's-Hertogenbosch, The Netherlands). The results were visualized by annotating them on a kinase phylogenetic tree using the web-based Coral tool (http://phanstiel-lab.med.unc.edu) (Metz, K. S., et al. Coral: Clear and Customizable Visualization of Human Kinome Data. Cell Syst 7, 347-350 e341 (2018)). Additionally, Ingenuity Pathway Analysis (Qiagen, Hilden, Germany) was used to perform gene enrichment analyses on the kinase activity profiles, utilizing the delta and unadjusted p-value of each peptide (two-sided two sample t-tests). Predicted pathway activation or predicted inhibition were generated by the z-score statistic, and further explored by the MAP tool (Qiagen, Hilden, Germany).

2. Results

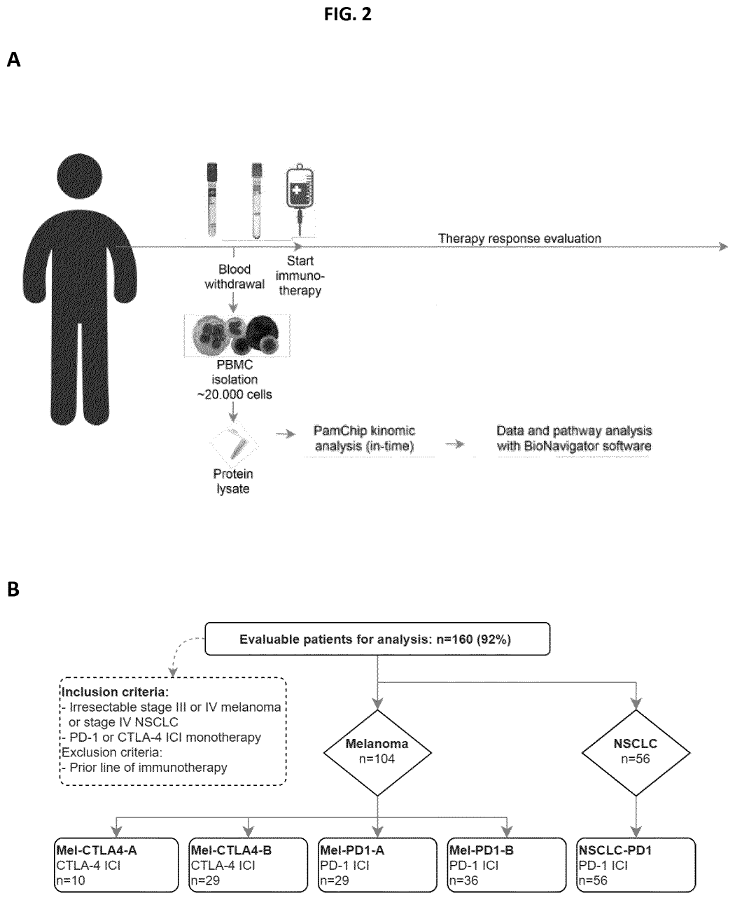

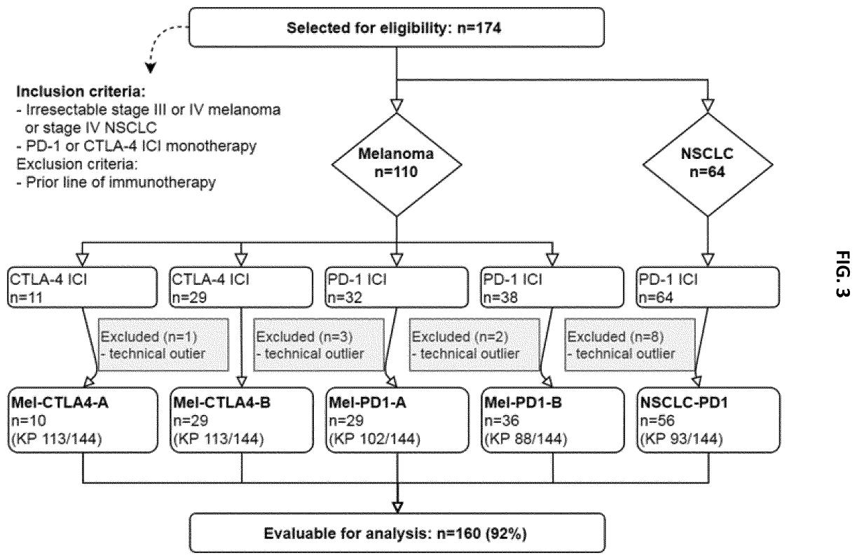

A total of 160 advanced cancer patients were evaluable for analysis (FIG. 2a-b). In 14 cases (8% of 174 patients) the patient samples had to be removed as they did not comply with the quality check (FIG. 3). The study protocols between the centers were different with regard to PBMC isolation protocol and the anti-coagulant used for blood collection. As a consequence, the different patient cohorts could not be pooled for the analysis, resulting in five distinct cohorts (FIG. 2a-b). The baseline patient characteristics are provided in Table 5. Patient cohort Mel-CTLA4-A was used as a training cohort for anti-CTLA-4 and consists of a group of 10 melanoma patients with an equal distribution of responders and non-responders. The patients had a baseline blood LDH level that was not elevated above 2× the upper limit of normal (ULN, i.e. <250 U/ml) to avoid bias based upon this independent prognostic factor. Similarly, the 29 patients in cohort Mel-PD1-A functioned as training cohort for anti-PD-1 with an equal distribution of responders (n=14) and non-responders (n=15). All other cohorts were used as cross-validation cohorts to assess the performance of response prediction by kinase activity profiling. The mean baseline blood LDH level was not elevated above 2×ULN in the Mel-PD1-A training cohort, although some individual patients did have an elevated LDH above this threshold. The mean baseline blood LDH level was higher and the range of values of individual patients larger in the cross-validation cohorts than in the training cohorts (Table 5).

TABLE 5
Baseline patient characteristics

| | Mel-CTLA4-A | | Mel-CTLA4-B | | Mel-PD1-A | | Mel-PD1-B | | NSCLC-PD1 | |
|---|---|---|---|---|---|---|---|---|---|---|
| Total, n | 10 | | 29 | | 29 | | 36 | | 56 | |
| Age, median (range) | 59.3 | (26-79) | 58.3 | (35-86) | 64.0 | (39-84) | 61.6 | (31-83) | 63.1 | (35-81) |
| Gender, n | | | | | | | | | | |
| Male | 4 | | 13 | | 16 | | 21 | | 36 | |
| Female | 6 | | 16 | | 13 | | 15 | | 20 | |
| Primary tumor, n | | | | | | | | | | |
| Melanoma | 10 | | 29 | | 29 | | 36 | | 0 | |
| NSCLC | 0 | | 0 | | 0 | | 0 | | 56 | |
| Adenocarcinoma | 0 | | 0 | | 0 | | 0 | | 37 | |
| SCC | 0 | | 0 | | 0 | | 0 | | 17 | |
| Large cell carcinoma | 0 | | 0 | | 0 | | 0 | | 2 | |
| Treatment regimen, n | | | | | | | | | | |
| Anti-PD1 | 0 | | 0 | | 29 | | 36 | | 56 | |
| Nivolumab | 0 | | 0 | | 1 | | 14 | | 50 | |
| Pembrolizumab | 0 | | 0 | | 28 | | 22 | | 6 | |
| Anti-CTLA4 | | | | | | | | | | |
| Ipilimumab | 10 | | 29 | | 0 | | 0 | | 0 | |
| Prior therapy lines, n (%) | | | | | | | | | | |
| 0 | 4 | (40%) | — | | 20 | (69%) | 30 | (83%) | 1 | (2%) |
| 1 | 4 | (40%) | — | | 9 | (31%) | 6 | (17%) | 46 | (82%) |
| 2 | 2 | (20%) | — | | 0 | (0%) | 0 | (0%) | 7 | (12%) |
| >2 | 0 | (0%) | — | | 0 | (0%) | 0 | (0%) | 2 | (4%) |
| Prior immunotherapy, n | | | | | | | | | | |
| No | 10 | | 29 | | 29 | | 36 | | 56 | |
| Yes | 0 | | 0 | | 0 | | 0 | | 0 | |
| Cerebral metastasis, n (%) | | | | | | | | | | |
| No | 6 | (60%) | — | | 17 | (59%) | 15 | (42%) | 0 | (0%) |
| Yes | 2 | (20%) | — | | 11 | (38%) | 2 | (5%) | 0 | (0%) |
| Unknown | 2 | (20%) | — | | 1 | (3%) | 19 | (53%) | 56 | (100%) |
| LDH (U/L), median (range) | 207 | (168-247) | 369 | (283-881) | 229 | (124-359) | 315 | (128-1523) | 269 | (133-860) |

Table 5: Baseline patient characteristics for each patient cohort. All patients received immune checkpoint inhibitor monotherapy and did not receive any prior line of immunotherapy. Abbreviations: lactate dehydrogenase (LDH), squamous cell carcinoma (SCC).

Kinase Activity Profiles of Anti-CTLA-4 Treated Patients

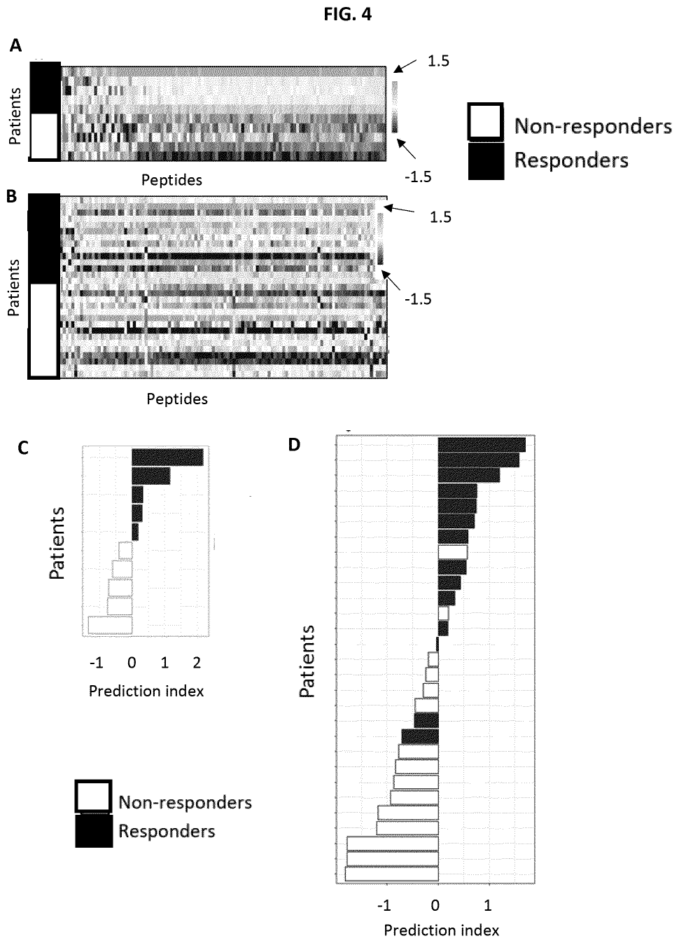

The correlation of the measured kinase activity profiles with treatment response is visualized for the training cohort Mel-CTLA4-A using a heat map (FIG. 4a). A profound difference in kinase activity was observed between the responder and non-responders. Generally, the signal of peptides is higher in responders compared to non-responders. For 83% of the target peptides, a significantly higher signal was found in responders compared to non-responders (two-sided two sample t-test, p-value <0.05; FDR <5%). This overall increase in kinase activity was confirmed in cohort Mel-CTLA4-B (FIG. 4b), although less pronounced since only 23% of the target peptides displayed a significantly higher signal in responders compared to non-responders (two-sided two sample t-test, p-value <0.05, FDR=18%). The relative increase in signal was higher in the Mel-CTLA4-A cohort (median Log 2 Fold Change=0.84; SD=0.15; ^{˜}80% increase) than in the Mel-CTLA4-B cohort (median Log 2 Fold Change=0.40; SD=0.17; ^{˜}30% increase). Hence, the increase in signal appears to reflect a systemic, non-specific, increase in signaling in the responders compared to the non-responders.

Kinase Activity Profiles of Anti-PD1 Treated Patients

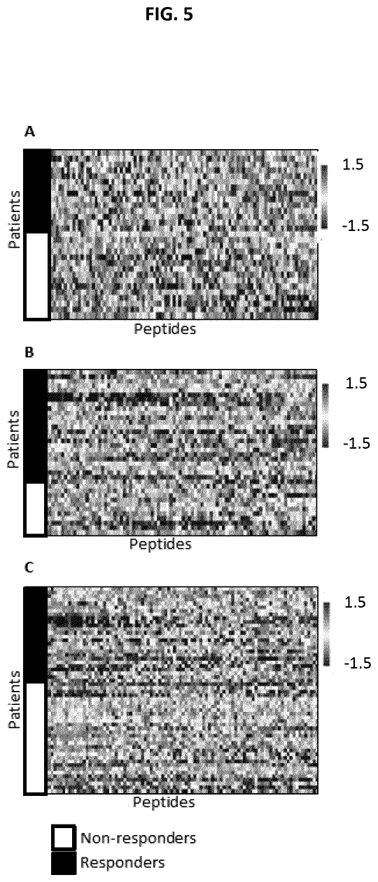

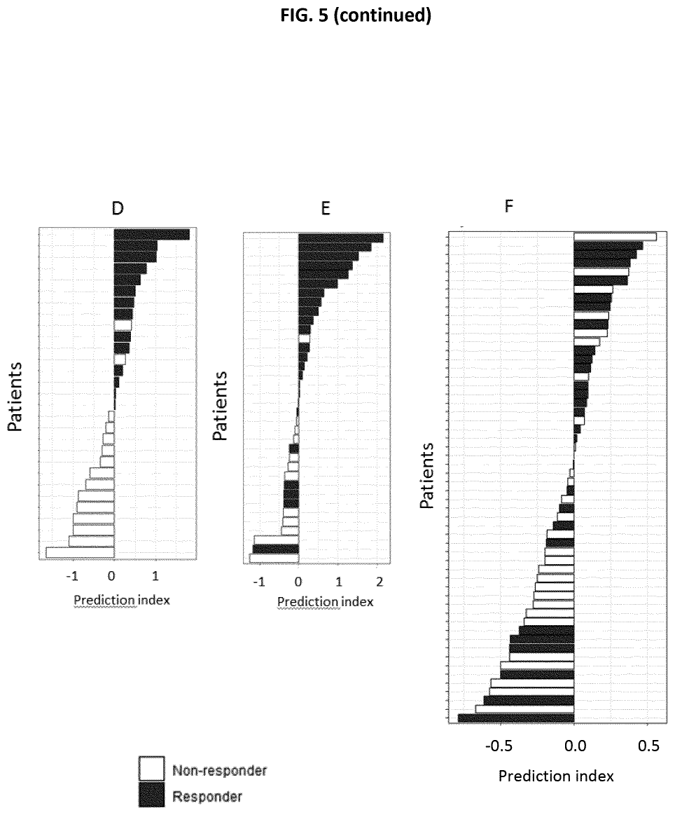

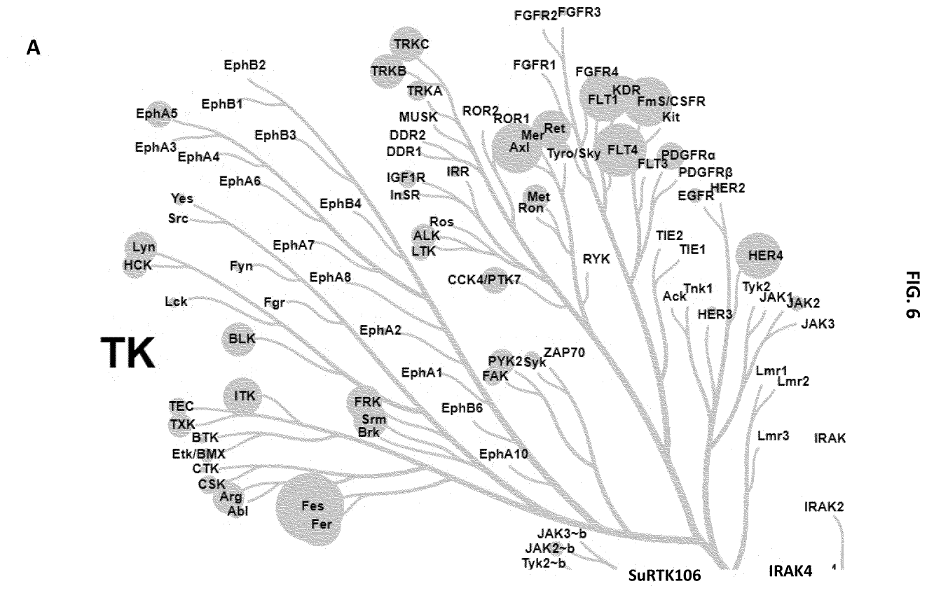

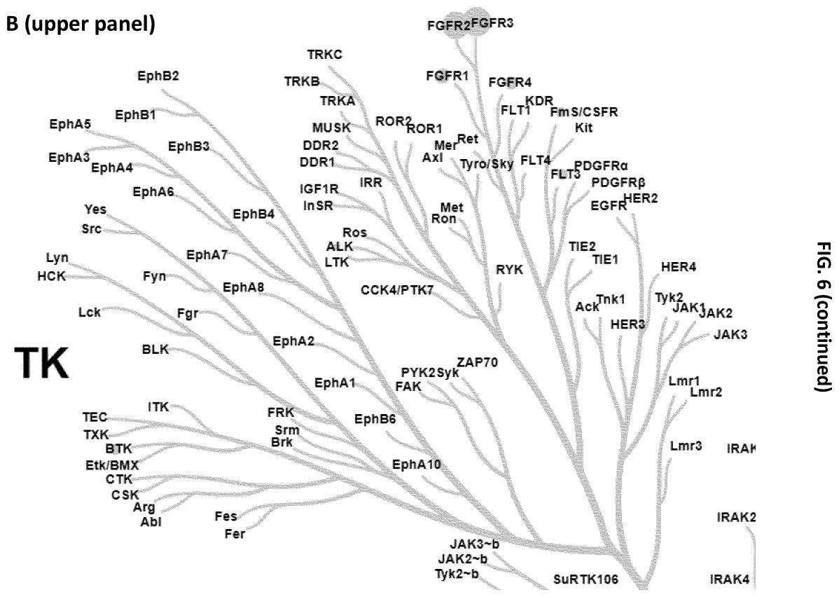

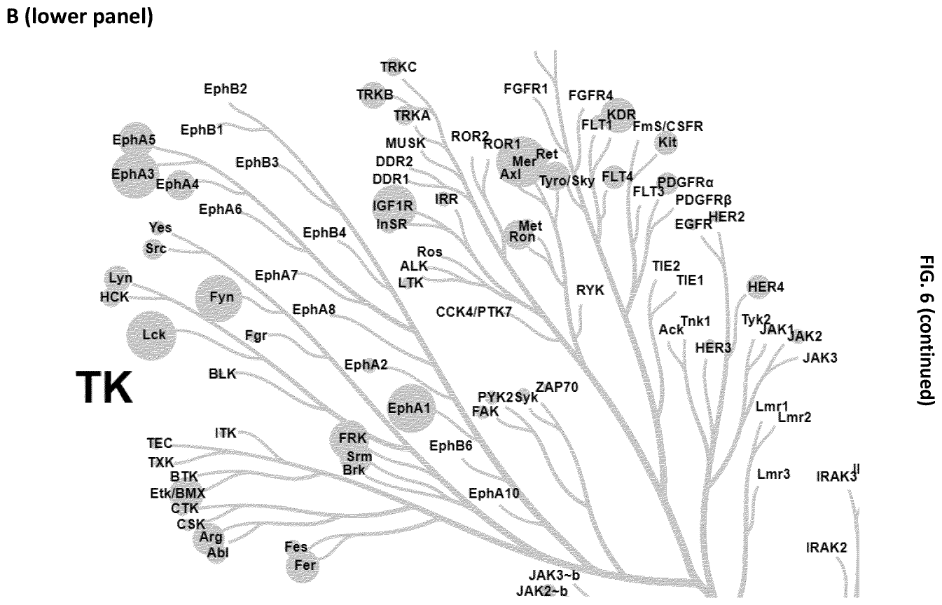

Training cohort Mel-PD1-A included melanoma patients who were treated with anti-PD-1. The data was normalized for overall kinase activity using the VSN method, since the data reflects differences in the ratio between peptides on the array rather than the overall differences. Significant differences were observed between responders and non-responders for cohort Mel-PD1-A (FIG. 5a). For 17% of the peptides in cohort Mel-PD1-A, a significant different signal was found in responders compared to non-responders (two-sided two sample t-test, p<0.05; FDR=29%). These differentially phosphorylated peptides represent both higher and lower signals in responders compared to non-responders. Likewise, differential peptide phosphorylation was observed in cohorts Mel-PD1-B and NSCLC-PD1, consisting of respectively melanoma and NSCLC patients who were treated with PD-1 ICIs (FIG. 5b-c). In cohort Mel-PD1-B, 16 peptides (18%) displayed significantly differential signals for response (two-sided two sample t-test, p<0.05, FDR=25%) whereas in cohort NSCLC-PD1, 19% of the peptides was significantly differently phosphorylated in responders compared to non-responders.

Response Classification by Kinase Activity Profile

To investigate the potential use of kinase activity profiling as a biomarker for response to ICI therapy, classification analysis was performed using the binary grouping of responders and non-responders. Because of the variation in the differentiating peptide phosphorylation profile between the CTLA-4 and PD-1 ICIs cohorts, a separate PLS-DA classification model was trained for each cohort and predictive scores for each patient were obtained using cross-validation. This resulted in a CCR of 100% (90% CI 74-100%) in the training cohort Mel-CTLA4-A and 83% (64-93%) in cross-validation cohort Mel-CTLA4-B comprising the groups of patients treated for melanoma with anti-CTLA4 ICIs (FIG. 4c,d). For the anti-PD-1 treated melanoma and NSCLC patients, the CCR was 93% (80-99%) in the training cohort Mel-PD1-A, 78% (63-88%) in cross-validation cohort Mel-PD1-B, and 68% (56-78%) in cross-validation cohort NSCLC-PD1 (FIG. 5d-f).

Upstream Kinase and Canonical Pathway Analysis

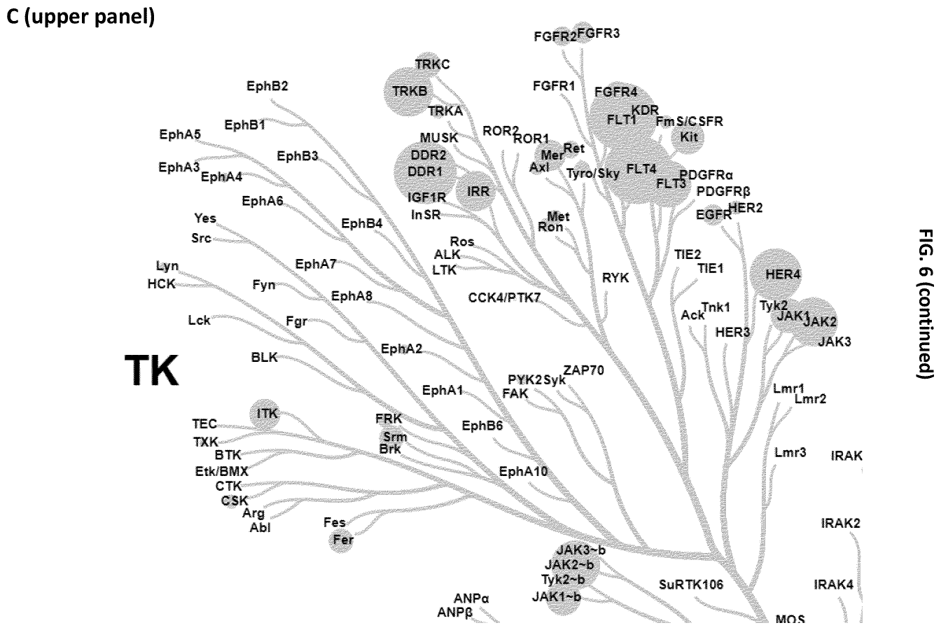

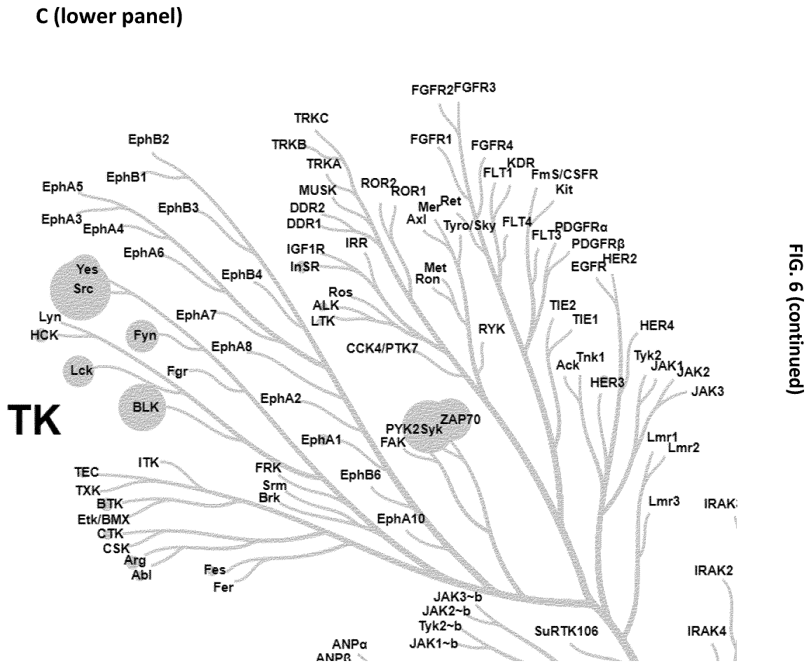

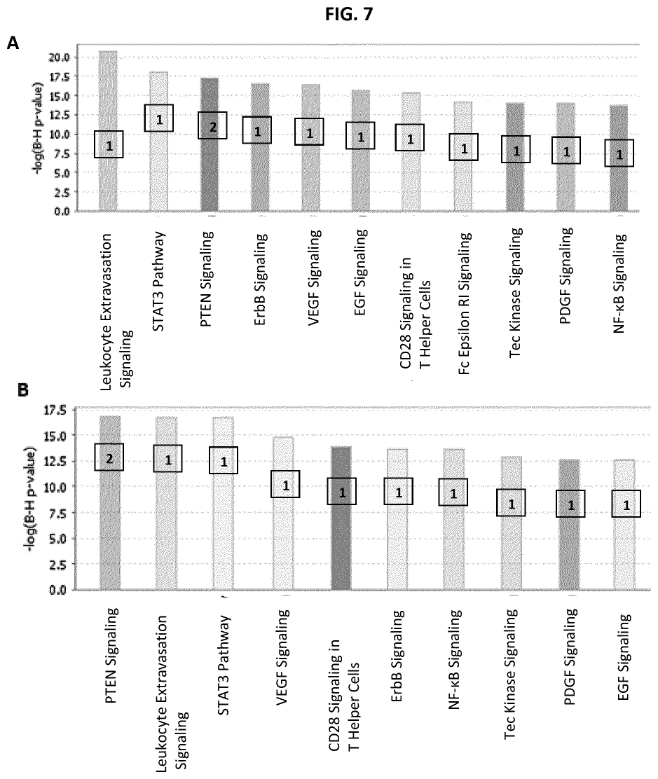

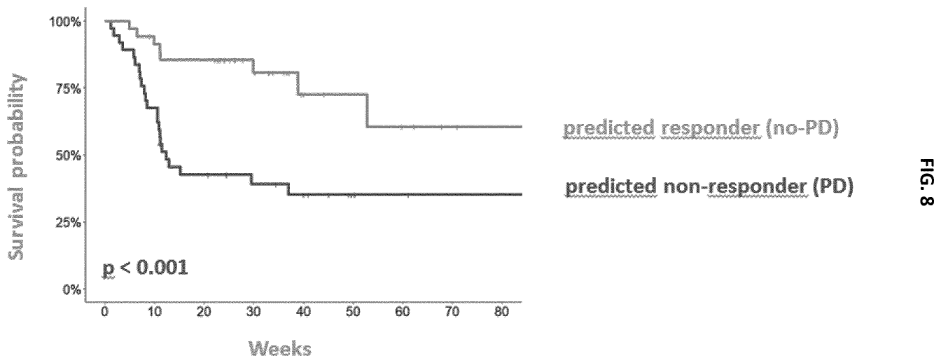

To interrogate the biological processes underlying response or resistance to ICIs, a bioinformatic approach was used to determine the upstream activated kinases. We focused on the anti-PD-1 cohorts as the overall increase of kinase activity in anti-CTLA-4 responders hampered the identification of the most relevant peptides. The results of this analysis were annotated to a phylogenetic tree for protein tyrosine kinases (FIG. 6). In the training cohort Mel-PD1-A, the VEGF family kinases and FES/FER were predicted to have relatively higher activity in responders compared to non-responders. Similarly, but less pronounced, the activity of Tyro-3, Axl and Mer kinase of the TAM-family and the Tropomyocin related kinases (TRK-family) were predicted to be positively correlated with response in this cohort. In the cross-validation cohort Mel-PD1-B, however, the activity of several kinases, including the Src family kinases, were negatively correlated with response. Interestingly, both these observations were corroborated in the cross-validation cohort NSCLC-PD1, where VEGF kinases were predicted to have higher activity and the Src family kinases to have lower kinase activity in responders compared to non-responders. A canonical pathway analysis focused on two anti-PD-1 cohorts Mel-PD1-A and NSCLC-PD1 revealed the importance of target molecules involved in immune cell migration/leukocyte extravasation and co-stimulation of T helper cells. In addition, increased kinase activity in the STAT3-, ErbB-, VEGF- and EGF-signaling pathways are related to response to therapy, whereas PTEN-pathway activation is associated with resistance to PD-1 ICIs (FIG. 7).

3. Discussion

This study demonstrates that the kinase activity of at least two kinases independently selected from within at least two families of kinases selected from the group consisting of the VEGFR or PDGFR family of kinases; the SRC family of kinases; the SYK family of kinases; and the TAM family of kinases; in a blood sample obtained from a patient allows predicting the response of said patient to a PD-1 or PD-L1 ICI.

### Example 2. Kinase Activity Profile Allows Predicting the Response of a Melanoma Patient in Need of an ICI to a Anti-PD1 ICI or a Combination of an Anti-PD1 and an Anti-CTLA4 ICI (Pred1ct-Mel Study)

1. Materials and Methods

Patient Population

72 Patients with irresectable stage Ill/IV advanced melanoma were included that were treated with anti-PD1 monotherapy (nivolumab or pembrolizumab; approximately 60% of the patients) or anti-PD1 and anti-CTLA4 combination therapy (nivolumab and ipilimumab; approximately 40% of the patients) as standard of care at the Isala Hospitial (Zwolle, The Netherlands), University Medical Center Utrecht (Utrecht, The Netherlands), Erasmus Medical Center (Rotterdam, The Netherlands), and Leiden University Medical Center (Leiden, The Netherlands). Patients that received any prior treatment with an ICI were excluded.

Preparation of PBMC Lysates

Venous blood of patients was collected using sodium-heparin as anticoagulant, and isolation of PBMCs was done on the same day at a centralized laboratory by density centrifugation and cryo-preserved until further use according to the study protocol.

Kinase Activity Profiling

Kinase activity profiling was performed at the ISO13485 certified Diagnostic Assay Services (DAS) laboratory at PamGene ('s-Hertogenbosch, The Netherlands) according to study protocols. For kinase activity profiling cryo-preserved PBMCs were thawed and lysed. Kinase activity profiles were measured on dedicated PamChip-4 PTK microarrays (PamGene, 's-Hertogenbosch, The Netherlands) in several runs using 4 PamStation-12 instruments in parallel. The runs were designed to contain 9 patient samples (4 replicates each) and 12 replicates of a fixed Internal Assay Control (IAC) sample.

Data Processing

Recorded images were quantified using standard methods in Bionavigator 6.3.67 ('s-Hertogenbosch, The Netherlands) interfaced to R 3.3.1 (R-project, www.rproject.org), including standard data quality checks to remove arrays that were defective or otherwise resulted in a clear outlier. The quantified data was normalized using the variance stabilizing normalization (VSN, W. Huber, A. von Heydebreck, H. Sultmann, A. Poustka, and M. Vingron, “Variance stabilization applied to microarray data calibration and to the quantification of differential expression.,” Bioinformatics, vol. 18 Suppl 1, pp. S96-104, 2002.) method and the transformed data was corrected for systematic effects between the runs by applying the ComBat method (W. E. Johnson, C. Li, and A. Rabinovic, “Adjusting batch effects in microarray expression data using empirical Bayes methods,” Biostatistics, vol. 8, no. 1, pp. 118-127, January 2007). Hereto, a ComBat correction model between the runs was calculated using the IAC sample and subsequently applied to the patient samples. In this way the correction depends on the IAC sample and not on the patient samples. As a result, this method can be used to correct for drift and batch effects in new measurements, i.e. when applying a predictive model based on the current measurements to new samples.

Data Analysis

The processed data were compared for the group of patients that showed Progressive Disease (PD) at or before the 24 week response evaluation performed as part of the hospitals' Standard of Care treatment protocol to the group of patients that did not. Evaluation of progression/response was performed at the hospitals according to RECIST (Eisenhauer E A, Therasse P, Bogaerts J, et al. New response evaluation criteria in solid tumours: revised RECIST guideline (version 1.1). Eur J Cancer. 2009; 45(2):228-247. doi:10.1016/j.ejca.2008.10.026). Univariate (per peptide) analysis was performed using a two-sided two sample t-test with binary grouping as a covariate (PD versus no-PD). Peptides with p<0.01 were regarded as significant. Classification analysis was performed using Elastic Net Classification using the R-package GLMnet 4.0 (J. Friedman, T. Hastie, and R. Tibshirani, “Regularization paths for generalized linear models via coordinate descent,” J. Stat. Softw., vol. 33, no. 1, pp. 1-22, February 2010), independent predictions for patients samples were obtained using cross-validation. The effect on Progression Free Survival (PFS) of stratifying the patients according to the obtained predictions was analyzed by standard methods for survival analysis in R 3.3.1 (R-project, www.rproject.org). A prediction of the kinases responsible for the changes in peptide phosphorylation in the kinomic profiles were obtained using the Upstream Kinase Analysis tool in Bionavigator 6.3.67 (PamGene, 's-Hertogenbosch, The Netherlands) and the results visualized by annotating them on a kinase phylogenetic using the Coral tool (http://phanstiel-lab.med.unc.edu/CORAL/).

2. Results

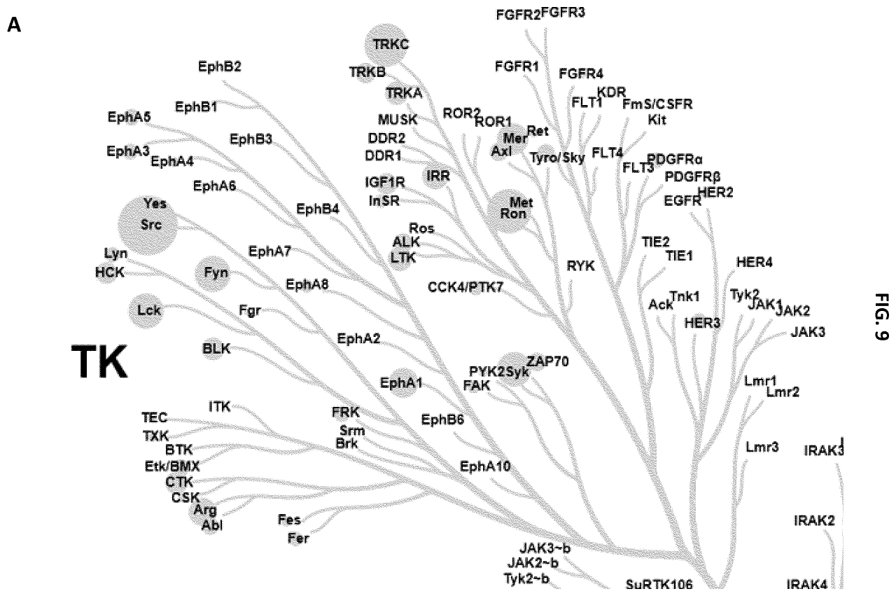

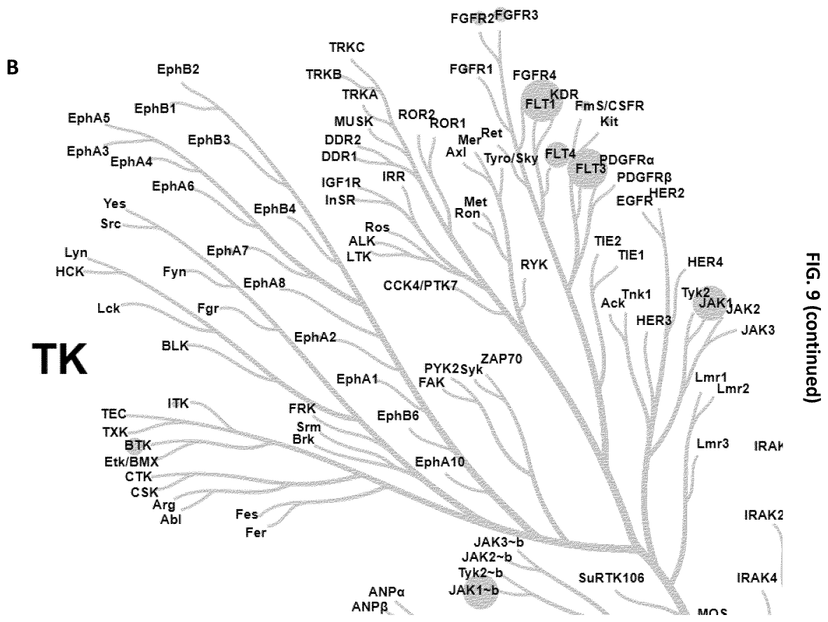

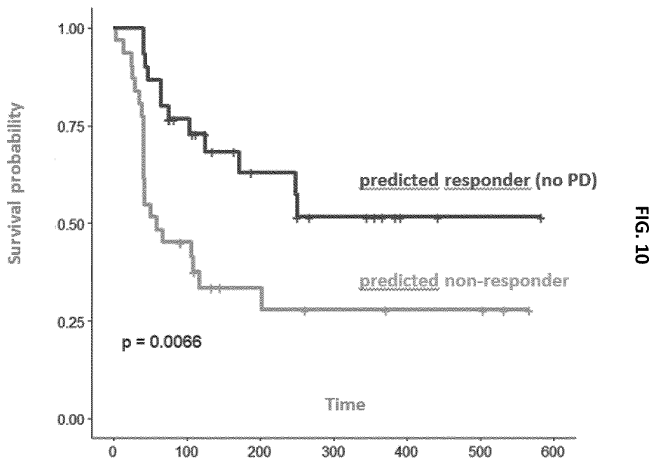

Significant differences in peptide phosphorylation where observed between the group with PD (no-responders) and the group with no PD (responders), see Table 6 for peptides with p<0.01. The classification analysis resulted in a correct classification rate of 71%. The effect of stratifying the patients according to model predictions is shown in FIG. 8. The group of patients for which no PD was predicted showed a significantly longer median PFS than the group of patients for who PD was predicted (>370 days vs. 86 days, HR=0.24 CI₉₅=0.1-0.56, p<0.001). FIG. 8A shows the kinase with a higher activity in the patients with PD (non-responders), most pronounced are Src family kinases (notably Src, Fyn, BLK, LCK), TRKC and RON. FIG. 8B shows the kinase with a higher activity in patients with no-PD (responders), these are VEGFR1 (FLT1), FLT3, VEGFR3 (FLT4), and JAK1.

TABLE 6

| Peptide ID | p-value | delta | signal higher in |
|---|---|---|---|
| RET_1022_1034 | 0.000437984 | −0.104194351 | PD (non-responder) |
| MK12_178_190 | 0.00071567 | 0.076973259 | No-PD (responder) |
| PECA1_706_718 | 0.000857333 | −0.204651952 | PD (non-responder) |
| MK07_211_223 | 0.000862799 | 0.083013602 | No-PD (responder) |
| VINC_815_827 | 0.000886112 | 0.162812233 | No-PD (responder) |
| CDK7_157_169 | 0.001591751 | 0.198949546 | No-PD (responder) |
| MK10_216_228 | 0.001779752 | 0.163573921 | No-PD (responder) |
| RBL2_99_111 | 0.001877899 | −0.122579321 | PD (non-responder) |
| ERBB4_1181_1193 | 0.001982704 | −0.154370129 | PD (non-responder) |
| ZBT16_621_633 | 0.002069873 | 0.195447952 | No-PD (responder) |
| ZAP70_485_497 | 0.003028194 | −0.144487262 | PD (non-responder) |
| EPHB1_771_783 | 0.003362769 | −0.155206129 | PD (non-responder) |
| FER_707_719 | 0.004326319 | −0.113440342 | PD (non-responder) |
| ANXA2_17_29 | 0.004655906 | −0.190498546 | PD (non-responder) |
| STAT4_714_726 | 0.005071104 | −0.189352557 | PD (non-responder) |
| FRK_380_392 | 0.005370284 | −0.103059381 | PD (non-responder) |
| VGFR3_1061_1073 | 0.005753078 | −0.096072115 | PD (non-responder) |
| JAK1_1015_1027 | 0.006396404 | −0.118198112 | PD (non-responder) |
| DCX_109_121 | 0.00730859 | 0.077001117 | No-PD (responder) |
| CDK2_8_20 | 0.007429449 | −0.149974361 | PD (non-responder) |
| INSR_1348_1360 | 0.007694244 | 0.165090129 | No-PD (responder) |
| MBP_259_271 | 0.008271371 | 0.155899286 | No-PD (responder) |
| ACHD_383_395 | 0.008371386 | −0.150277853 | PD (non-responder) |
| ODPAT_291_303 | 0.008403396 | 0.082908414 | No-PD (responder) |
| LCK_387_399 | 0.009663599 | −0.102207296 | PD (non-responder) |

3. Discussion

This study demonstrates that the kinase activity of at least two kinases independently selected from within at least two families of kinases selected from the group consisting of the VEGFR or PDGFR family of kinases; the SRC family of kinases; or the JAK family of kinases, in a blood sample obtained from a patient allows predicting the response of said patient to a PD-1 or PD-L1 ICI and combination therapy with a PD-1 and anti-CTLA4 ICI.

### Example 3. Kinase Activity Profile Allows Predicting the Response of a NSCLC Patient in Need of an ICI to an Anti-PD1 ICI or a Combination of an Anti-PD1 ICI with Chemotherapy (Pred1ct-Nsclc Study)

1. Materials and Methods

Patient Population

64 Patients with irresectable stage IV advanced non small cell lung cancer (NSCLC) were included that were treated for the first time with anti-PD1 CI at the UMC Groningen (Groningen, The Netherlands), Radboud UMC (Nijmegen, The Netherlands), and Erasmus MC (Rotterdam, The Netherlands). Patients with tumours showing high (≥50%) and low (<50%) PD-L1 expression were included. Typically, patients with high PD-L1 expression received anti-PD1 monotherapy (pembrolizumab) whereas patients with low PD-L1 expression received anti-PD1 monotherapy (nivolumab, pembrolizumab) or anti-PD1 therapy in combination with chemotherapy.

Preparation of PBMC Lysates

Venous blood of patients was collected shortly before start of treatment (baseline) using sodium-heparin as anticoagulant, and isolation of PBMCs was done on the same day at a centralized laboratory by density centrifugation and cryo-preserved until further use according to the study protocol.

Kinase Activity Profiling

Kinase activity profiling was performed at the ISO13485 certified Diagnostic Assay Services (DAS) laboratory at PamGene ('s-Hertogenbosch, The Netherlands) according to study protocols. For kinase activity profiling cryo-preserved cells were thawed and lysed. Kinase activity profiles were measured on dedicated PamChip-4 PTK microarrays in several runs using 4 PamStation-12 instruments in parallel. The runs were designed to contain 9 patient samples (4 replicates each) and 12 replicates of a fixed Internal Assay Control (IAC) sample.

Data Processing

Recorded images were quantified using standard methods in Bionavigator 6.3.67 ('s-Hertogenbosch, The Netherlands) interfaced to R 3.3.1 (R-project, www.rproject.org), including standard data quality checks to remove arrays that were defective or otherwise resulted in a clear outlier. The quantified data was normalized using the VSN method (W. Huber, A. von Heydebreck, H. Sultmann, A. Poustka, and M. Vingron, “Variance stabilization applied to microarray data calibration and to the quantification of differential expression.,” Bioinformatics, vol. 18 Suppl 1, pp. S96-104, 2002) and the transformed data was corrected for systematic effects between the runs by applying the ComBat method (W. E. Johnson, C. Li, and A. Rabinovic, “Adjusting batch effects in microarray expression data using empirical Bayes methods,” Biostatistics, vol. 8, no. 1, pp. 118-127, January 2007). Hereto, a ComBat correction model between the runs was calculated using the IAC sample and subsequently applied to the patient samples. In this way the correction depends on the IAC sample and not on the patient samples. As a result, this method can be used to correct for drift and batch effects in new measurements, i.e. when applying a predictive model based on the current measurements to new samples.

Data Analysis

The processed data was compared for the group of patients that showed Progressive Disease (PD) at or before the 12 week response evaluation performed as part of the hospitals' Standard of Care treatment protocol to the group of patients that did not. Evaluation of progression/response was performed at the hospitals according to RECIST. Univariate (per peptide) analysis was performed using a two-sided two sample t-test with binary grouping as a covariate (PD versus no-PD). Peptides with p<0.01 were regarded as significant. Classification analysis was performed using Partial Least Squares Discriminant Analysis essentially as described in a previous publication (S. Arni et al., “Ex vivo multiplex profiling of protein tyrosine kinase activities in early stages of human lung adenocarcinoma,” Oncotarget, vol. 8, no. 40, pp. 68599-68613, September 2017), independent predictions for patients samples were obtained using cross-validation. The effect on Progression Free Survival (PFS) of stratifying the patients according to the obtained predictions was analyzed by standard methods for survival analysis in R 3.3.1. A prediction of the kinases responsible for the changes in peptide phosphorylation in the kinomic profiles were obtained using the Upstream Kinase Analysis tool in Bionavigator 6.3.67 (PamGene, 's-Hertogenbosch, The Netherlands) and the results visualized by annotating them on a kinase phylogenetic using the Coral tool (K. S. Metz et al., “Coral: Clear and Customizable Visualization of Human Kinome Data,” Cell Syst., vol. 7, no. 3, pp. 347-350.e1, September 2018) (http://phanstiel-lab.med.unc.edu/CORAL/).

2. Results

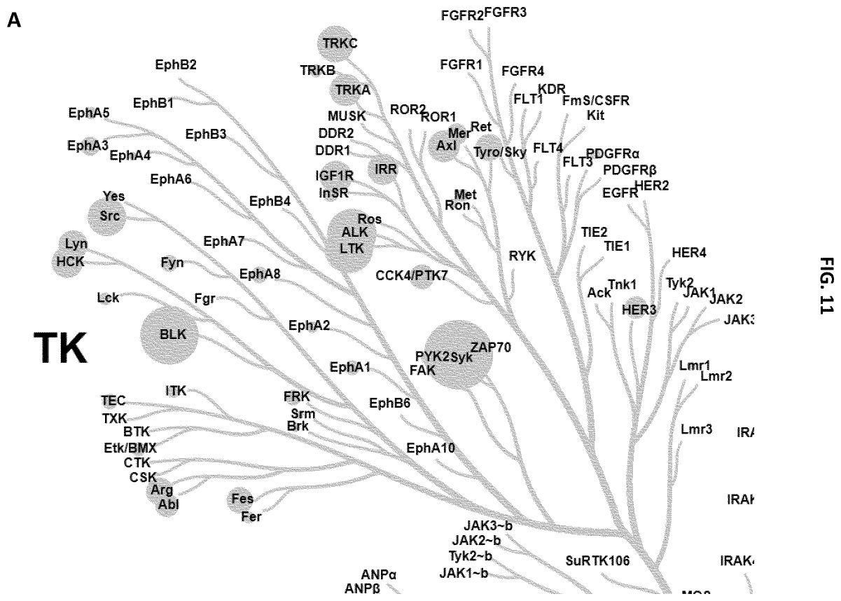

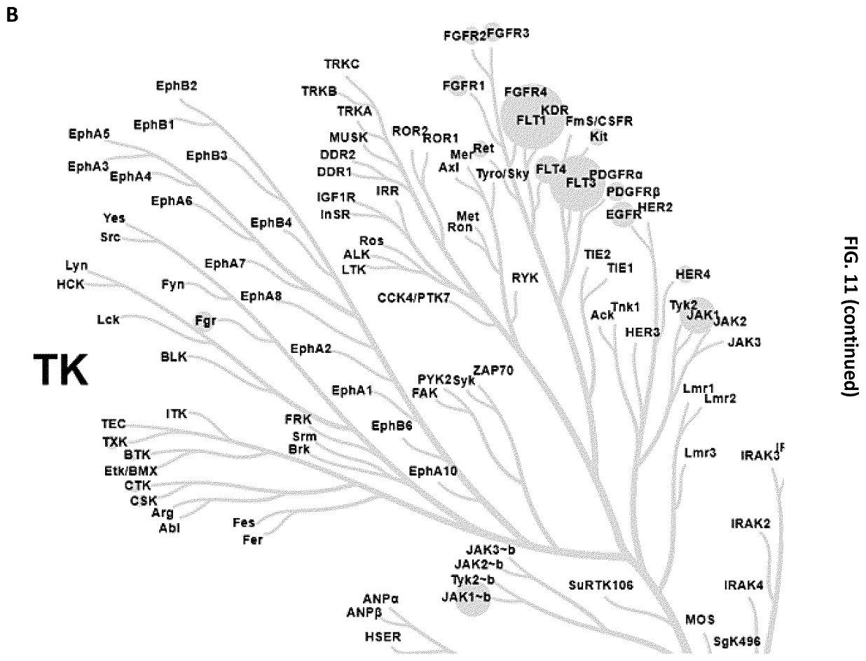

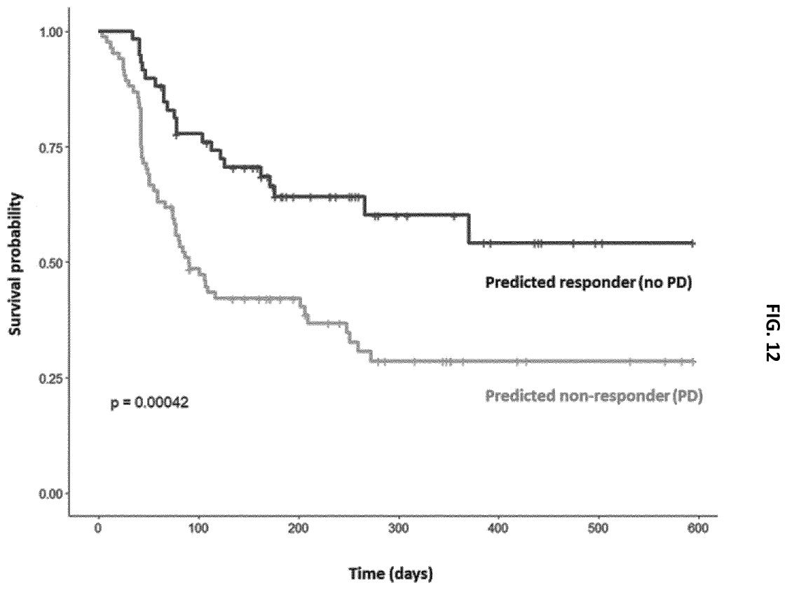

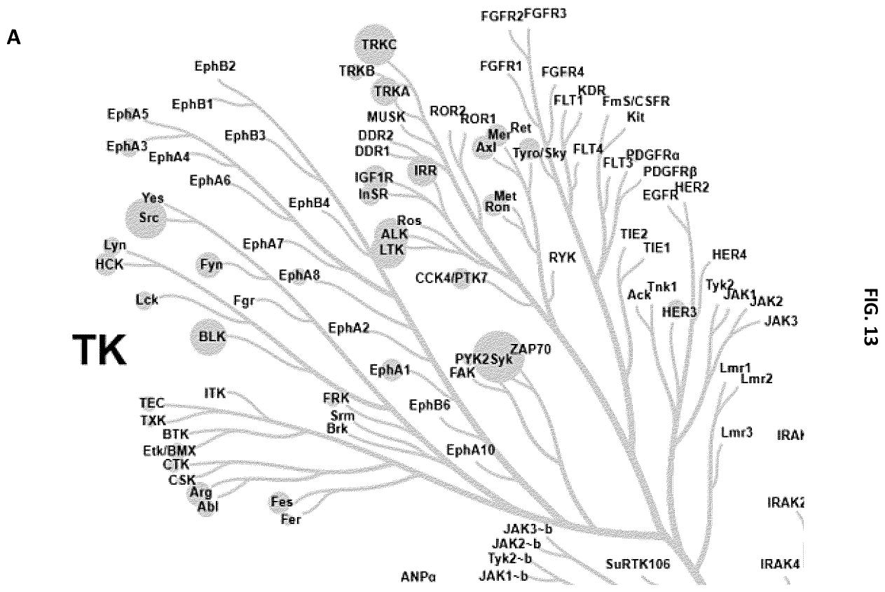

Significant differences in peptide phosphorylation where observed between the group with PD (no-responders) and the group with no PD (responders), see Table 7 for peptides with p<0.01. The classification analysis resulted in a correct classification rate of 70%. The effect of stratifying the patients according to model predictions is shown in FIG. 10. The group of patients for which no PD was predicted showed a significantly longer median PFS than the group of patients for who PD was predicted (>171 days vs. 59 days, HR=0.39 CI₉₅=0.19-0.79, p=0.007). FIG. 11A shows the kinase with a higher activity in the patients with PD (non-responders), most pronounced is the signal for Syk and the Src family kinase BLK, other Src family kinases and ALK, LTK. FIG. 11B shows the kinase with a higher activity in patients with no-PD (responders), most pronounced are VEGFR1 (FLT1) and FLT3.

3. Discussion

This study demonstrates that the kinase activity of at least two kinases independently selected from within at least two families of kinases selected from the group consisting of the VEGFR family of kinases; the SRC family of kinases; the SYK family, or the ALK/LTK family of kinases in a blood sample obtained from a patient allows predicting the response of said patient to a PD-1 or PD-L1 ICI and combination therapy with a PD-1 CI and chemotherapy.

TABLE 7

| unicol | p-value | delta | Signal higher in |
|---|---|---|---|
| VGFR2_989_1001 | 0.001674418 | −0.174391687 | PD(non-responder) |
| EGFR_862_874 | 0.001892488 | −0.228330165 | PD(non-responder) |
| VINC_815_827 | 0.002074327 | 0.171527088 | No-PD (responder) |
| EPHA1_774_786 | 0.002412647 | −0.142027542 | PD(non-responder) |
| TNNT1_2_14 | 0.002508069 | 0.4059048 | No-PD (responder) |
| JAK2_563_577 | 0.002680417 | −0.113481469 | PD(non-responder) |
| EGFR_1103_1115 | 0.002954093 | −0.17872712 | PD(non-responder) |
| EPHB1_771_783 | 0.003054618 | −0.158481404 | PD(non-responder) |
| EPOR_419_431 | 0.003968014 | −0.315531433 | PD(non-responder) |
| PTN11_539_551 | 0.004001196 | 0.261296332 | No-PD (responder) |
| PGFRB_771_783 | 0.004011602 | −0.181727469 | PD(non-responder) |
| CD3Z_116_128 | 0.004264721 | −0.268122882 | PD(non-responder) |
| DYR1A_312_324 | 0.006025424 | 0.149698257 | No-PD (responder) |
| FES_706_718 | 0.006714904 | −0.151004925 | PD(non-responder) |
| PGFRB_768_780 | 0.007629552 | −0.15368703 | PD(non-responder) |
| EPOR_361_373 | 0.008235824 | −0.265711665 | PD(non-responder) |
| EPHA4_589_601 | 0.008430195 | 0.084007442 | No-PD (responder) |
| VGFR2_944_956 | 0.008770701 | 0.291703343 | No-PD (responder) |
| JAK1_1015_1027 | 0.009386425 | −0.103091389 | PD(non-responder) |
| PRRX2_202_214 | 0.009629248 | −0.075899884 | PD(non-responder) |
| VGFR1_1049_1061 | 0.009773116 | 0.177235097 | No-PD (responder) |

### Example 4. Kinase Activity Profile Allows Predicting the Response of Patient in Need of an ICI to a Anti-PD1 ICI

1. Materials and Methods

### Background

The data of example 2 (melanoma) and example 3 (NSCLC) shows limited overlap in the tables with significant peptides (p<0.01), but does show overlap in the kinase activities that may be used to predict ICI response. Here we combine both data sets to show that kinase activity profiles may be used to predict the response to ICI regardless of disease type.

Data Processing

Data processing was performed in the same way as for example 2 and example 3 but for now for the combined data set.

Data Analysis

See example 2 and example 3. The processed data was compared for the group of patients that showed Progressive Disease (PD) at or before the 12 week response evaluation or 24 weeks response evaluation for NSCLC and melanoma, respectively. This difference in time of evaluation reflects overall shorter PFS observed for NSCLC patients compared to that of melanoma patients. For classification analysis both elastic net classification and PLS-DA classification as applied, with essentially equal result.

2. Results

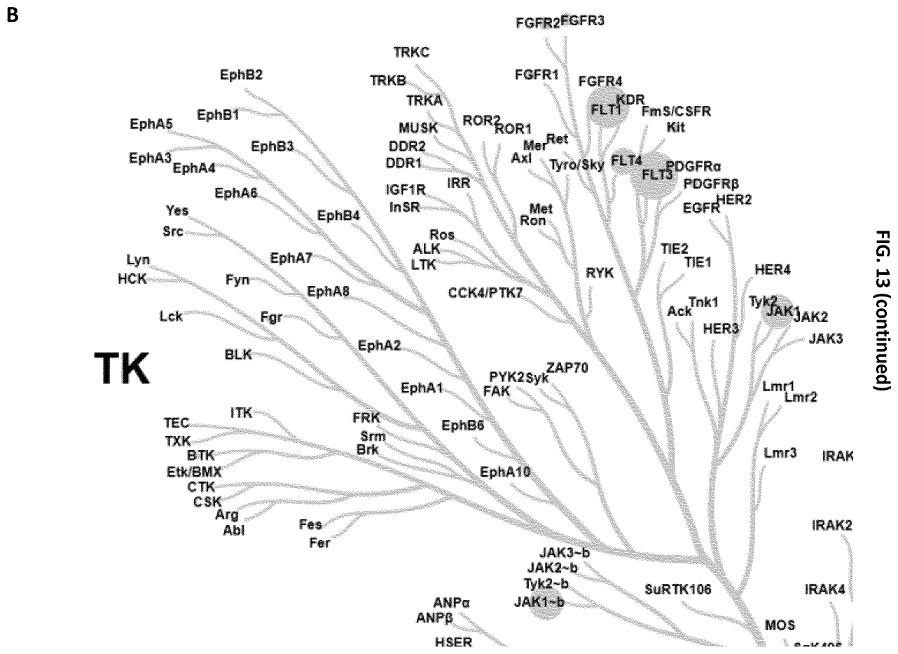

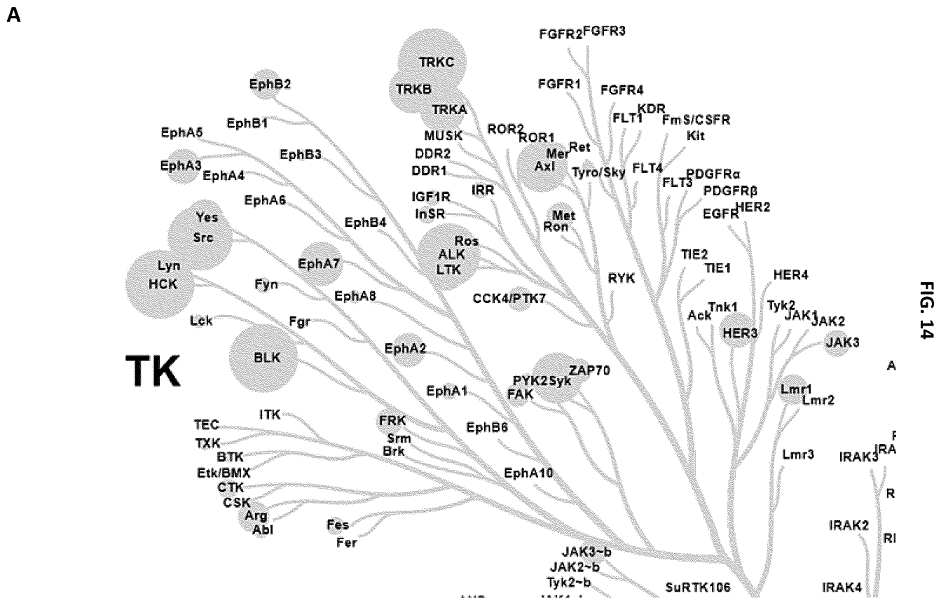

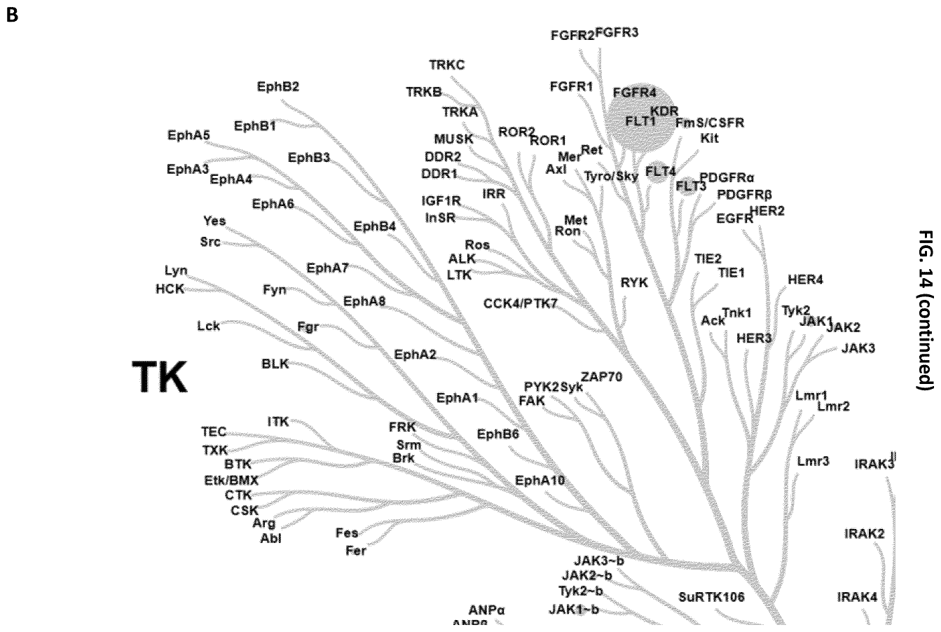

Significant differences in peptide phosphorylation where observed between the group with PD (no-responders) and the group with no PD (responders) with a significance that was stronger than that observed with the cohorts separately, see Table 8 for peptides with p<0.01. The classification analysis resulted in a correct classification rate of 65%. The effect of stratifying the patients according to model predictions is shown in FIG. 12. The group of patients for which no PD was predicted showed a significantly longer median PFS than the group of patients for who PD was predicted (>266 days vs. 90 days, HR=0.42 CI₉₅=0.26-0.69 p<0.001). FIG. 13A shows the kinase with a higher activity in the patients with PD (non-responders), most pronounced is the signal for Syk, Src family kinases, ALK and LTK, and TRKC. FIG. 13B shows the kinases with a higher activity in patients with no-PD (responders), most pronounced are VEGFR1 (FLT1), FLT3, and JAK1.

3. Discussion

This study demonstrates that the kinase activity of at least two kinases independently selected from within at least two families of kinases selected from the group consisting of the VEGFR family of kinases; the SRC family of kinases; the SYK family, or the ALK/LTK family of kinases in a blood sample obtained from a patient allows predicting the response of said patient to a PD-1 or PD-L1 ICI and combination therapy with a PD-1 and CTLA4 ICI.

TABLE 8

| ID | p value | delta | Signal higher in |
|---|---|---|---|
| VINC_815_827 | 0.00002 | −0.164349973 | No-PD (responder) |
| EPHB1_771_783 | 0.00002 | 0.154836565 | PD (non-responder) |
| ERBB4_1181_1193 | 0.00008 | 0.124435298 | PD (non-responder) |
| RET_1022_1034 | 0.00011 | 0.089063458 | PD (non-responder) |
| EGFR_862_874 | 0.00011 | 0.162792966 | PD (non-responder) |
| CDK7_157_169 | 0.00012 | −0.204582646 | No-PD (responder) |
| JAK1_1015_1027 | 0.00012 | 0.112083361 | PD (non-responder) |
| FES_706_718 | 0.00013 | 0.150170103 | PD (non-responder) |
| CD3Z_116_128 | 0.00023 | 0.217625052 | PD (non-responder) |
| ZBT16_621_633 | 0.00024 | −0.148435414 | No-PD (responder) |
| PTN11_539_551 | 0.00025 | −0.205011383 | No-PD (responder) |
| FER_707_719 | 0.00028 | 0.097744986 | PD (non-responder) |
| MBP_259_271 | 0.00033 | −0.144607857 | No-PD (responder) |
| VGFR1_1049_1061 | 0.00035 | −0.141922399 | No-PD (responder) |
| NCF1_313_325 | 0.00040 | −0.301456273 | No-PD (responder) |
| CRK_214_226 | 0.00048 | 0.136511162 | PD (non-responder) |
| EPHA2_765_777 | 0.00049 | 0.102242939 | PD (non-responder) |
| JAK2_563_577 | 0.00056 | 0.100465454 | PD (non-responder) |
| VGFR2_944_956 | 0.00062 | −0.213109836 | No-PD (responder) |
| PP2AB_297_309 | 0.00065 | −0.131924033 | No-PD (responder) |
| C1R_199_211 | 0.00066 | −0.120068356 | No-PD (responder) |
| DYR1A_312_324 | 0.00070 | −0.103940666 | No-PD (responder) |
| B3AT_39_51 | 0.00072 | 0.093756668 | PD (non-responder) |
| STAT4_714_726 | 0.00087 | 0.139392257 | PD (non-responder) |
| VGFR2_989_1001 | 0.00104 | 0.113966227 | PD (non-responder) |
| EPHA4_589_601 | 0.00107 | −0.066987723 | No-PD (responder) |
| EPHA1_774_786 | 0.00108 | 0.101611562 | PD (non-responder) |
| EPOR_361_373 | 0.00123 | 0.215386868 | PD (non-responder) |
| K2C6B_53_65 | 0.00160 | −0.10516528 | No-PD (responder) |
| MK10_216_228 | 0.00161 | −0.115516186 | No-PD (responder) |
| RB_804_816 | 0.00162 | −0.125748187 | No-PD (responder) |
| PRGR_786_798 | 0.00174 | 0.068109795 | PD (non-responder) |
| EPHA7_607_619 | 0.00185 | 0.10853608 | PD (non-responder) |
| LCK_387_399 | 0.00195 | 0.086330548 | PD (non-responder) |
| RBL2_99_111 | 0.00201 | 0.077619262 | PD (non-responder) |
| MET_1227_1239 | 0.00218 | 0.061908875 | PD (non-responder) |
| ANXA2_17_29 | 0.00231 | 0.130832687 | PD (non-responder) |
| PECA1_706_718 | 0.00233 | 0.126119494 | PD (non-responder) |
| TYRO3_679_691 | 0.00288 | −0.093781576 | No-PD (responder) |
| MK01_180_192 | 0.00309 | −0.096198007 | No-PD (responder) |
| PAXI_111_123 | 0.00565 | 0.10258548 | PD (non-responder) |
| VGFR1_1206_1218 | 0.00565 | −0.051487133 | No-PD (responder) |
| CDK2_8_20 | 0.00591 | 0.107710078 | PD (non-responder) |
| CTNB1_79_91 | 0.00647 | 0.079826094 | PD (non-responder) |
| EPOR_419_431 | 0.00677 | 0.180520415 | PD (non-responder) |
| EGFR_1165_1177 | 0.00720 | −0.159544021 | No-PD (responder) |
| DCX_109_121 | 0.00728 | −0.080863692 | No-PD (responder) |
| EPHB4_583_595 | 0.00812 | −0.062383961 | No-PD (responder) |
| P85A_600_612 | 0.00901 | 0.162591949 | PD (non-responder) |

### Example 5. Kinase Activity Profile Allows Predicting the Response of an Ovarian Cancer Patient in Need of an ICI to an Anti-PD-L1ICI

1. Materials and Methods

Patient Population

45 patients with Ovarian Cancer were treated with PD-L1 ICI durvalumab.

Preparation of PBMC Lysates

Venous blood of patients was collected shortly before start of treatment (baseline) using EDTA as anticoagulant, isolation of PBMCs was performed by density centrifugation per the institute's protocol.

Kinase Activity Profiling

Kinase activity profiling was performed at the ISO13485 certified Diagnostic Assay Services (DAS) laboratory at PamGene ('s-Hertogenbosch, The Netherlands) according to study protocols. For kinase activity profiling cryo-preserved cells were thawed and lysed. Kinase activity profiles were measured on dedicated PamChip-4 PTK microarrays in several runs using 4 PamStation-12 instruments in parallel. The runs were designed to contain 9 patient samples (4 replicates each) and 12 replicates of a fixed Internal Assay Control (IAC) sample.

Data Processing

Recorded images were quantified using standard methods in Bionavigator 6.3.67 ('s-Hertogenbosch, The Netherlands) interfaced to R 3.3.1 (R-project, www.rproject.org), including standard data quality checks to remove arrays that were defective or otherwise resulted in a clear outlier. The quantified data was normalized using the VSN method (W. Huber, A. von Heydebreck, H. Sultmann, A. Poustka, and M. Vingron, “Variance stabilization applied to microarray data calibration and to the quantification of differential expression.,” Bioinformatics, vol. 18 Suppl 1, pp. S96-104, 2002) and the transformed data was corrected for systematic batch effects between the runs and the two sample batches by applying the ComBat method (W. E. Johnson, C. Li, and A. Rabinovic, “Adjusting batch effects in microarray expression data using empirical Bayes methods,” Biostatistics, vol. 8, no. 1, pp. 118-127, January 2007).

Data Analysis

The processed data was compared for the group of patients that showed Progressive Disease (PD=Non-responder) as Best Overall Response (BOR) compared the group Stable Disease, Partial Response, Complete Response (SD, PR, CR=Responder), as reported by the hospital. Univariate (per peptide) analysis was performed using a two-sided two sample t-test with binary grouping as a covariate (PD versus no-PD). Peptides with p<0.01 were regarded as significant. Classification analysis was performed using Partial Least Squares Discriminant Analysis essentially as described in a previous publication (S. Arni et al., “Ex vivo multiplex profiling of protein tyrosine kinase activities in early stages of human lung adenocarcinoma,” Oncotarget, vol. 8, no. 40, pp. 68599-68613, September 2017), independent predictions for patients samples were obtained using cross-validation. A prediction of the kinases responsible for the changes in peptide phosphorylation in the kinomic profiles were obtained using the Upstream Kinase Analysis tool in Bionavigator 6.3.67 (PamGene, 's-Hertogenbosch, The Netherlands) and the results visualized by annotating them on a kinase phylogenetic using the Coral tool (K. S. Metz et al., “Coral: Clear and Customizable Visualization of Human Kinome Data,” Cell Syst., vol. 7, no. 3, pp. 347-350.e1, September 2018) (http://phanstiel-lab.med.unc.edu/CORAL/).

2. Results

Significant differences in peptide phosphorylation where observed between the Responders and Non-responders, see Table 9 for peptides with p<0.01. The classification analysis resulted in a correct classification rate of 70%. FIG. 14A shows in a phylogenetic tree that the kinase with a higher activity in the patients with PD (non-responders), most pronounced is the signal for the Src family kinases BLK, HCK, and Src, and ALK, LTK, TRKC, TRKB. FIG. 14B shows the kinase with a higher activity in Responders, most pronounced is VEGFR1 (FLT1).

3. Discussion

This study demonstrates that the kinase activity of at least two kinases independently selected from within at least two families of kinases selected from the group consisting of the VEGFR family of kinases; the SRC family of kinases; the ALK/LTK family of kinases in a blood sample obtained from a patient allows predicting the response of said patient to a PD-L1 ICI.

TABLE 9

| unicol | p | delta | Higher signal in |
|---|---|---|---|
| PTN11_539_551 | 0.002 | −0.19 | Responder |
| PTN11_541_551 | 0.004 | −0.15 | Responder |
| SRC_524_536 | 0.008 | 0.26 | Non-responder |
| NCF1_313_325 | 0.008 | −0.27 | Responder |
| MBP_198_210 | 0.009 | −0.17 | Responder |
| MUSK_548_560 | 0.010 | −0.13 | Responder |

1. A method for predicting the response of a patient in need of an immune checkpoint inhibitor (ICI), to treatment with a programmed cell death-1 (PD-1) or programmed death-ligand 1 (PD-L1) ICI, comprising the steps of:
(a) measuring the kinase activity of at least two kinases independently selected from within at least two families of kinases selected from the group consisting of:
the vascular endothelial growth factor receptor (VEGFR) or platelet-derived growth factor receptor (PDGFR) family of kinases;
the SRC family of kinases;
the spleen tyrosine kinase (SYK) family of kinases;
the TAM family of kinases;
the janus kinase (JakA) family of kinases; and
the anaplastic lymphoma kinase (ALK) family of kinases,
in a blood sample obtained from said patient, thereby providing a kinase activity profile of said blood sample, preferably a blood sample comprising peripheral blood mononuclear cells; and
(b) predicting from said kinase activity profile the response of said patient to treatment with said PD-1 or PD-L1 ICI.

2. The method according to claim 1, wherein
the VEGFR or PDGFR family of kinases consists of VEGFR1, VEGFR2, VEGFR3, PDGFRalpha, PDGFRbeta, macrophage colony-stimulating factor 1 receptor (CSF-1R), Kit, FMS-like receptor tyrosine kinase-3 (FLT3), and any combination thereof; preferably the VEGFR or PDGFR family of kinases consists of VEGFR1, VEGFR3, FLT3, and any combination thereof,
the SRC family of kinases consists of SRC, YES, FYN, FGR, lymphocyte cell-specific protein-tyrosine kinase (LCK), hemopoietic cell kinase (HCK), B lymphoid tyrosine kinase (BLK), LYN, Fyn-related kinase (FRK), and any combination thereof, preferably the SRC family of kinases consists of SRC, FYN, BLK and any combination thereof,
the SYK family of kinases consists of SYK, zeta chain of T cell receptor associated protein kinase 70 (ZAP-70), and any combination thereof,
the TAM family of kinases consists of TYRO3, AXL, MER proto-oncogene, tyrosine kinase (MERTK), and any combination thereof,
the JakA family of kinases consists of JAK1, JAK2, JAK3, TYK2 and any combination thereof; and/or
the ALK family of kinases consists of ALK, leukocyte receptor tyrosine kinase (LTK), and any combination thereof.

3. The method according to claim 1, wherein said method comprises measuring the kinase activity of at least two kinases selected from the group consisting of VEGFR1, VEGFR3, FLT3, SRC, FYN, BLK, SYK, ZAP70, TYRO-3, AXL, MER, TRKC, RON, ALK, LTK, and JAK1.

4. The method according to claim 1, wherein said PD-1 or PD-L1 ICI is an antibody.

5. The method according to claim 1, said PD-1 or PD-L1 ICI is selected from the group consisting of Nivolumab, Pembrolizumab, Durvalumab, Atezolizumab, Avelumab, Cemiplimab, Camrelizumab, Sintilimab, Tislelizumab, Toripalimab, and any combination thereof, preferably selected from the group consisting of Nivolumab, Prembrolizumab, and any combination thereof optionally in combination with a compound selected from the group consisting of Ipilimumab or Tremilimumab.

6. The method according to claim 1, said treatment with said PD-1 or PD-L1 ICI is combined with an antineoplastic treatment selected from the group consisting of chemotherapy, radiotherapy, chemoradiotherapy, surgery, an immune checkpoint inhibitor, a kinase inhibitor, a cancer vaccine, an antibody-drug conjugate, a nuclear receptor agonist, a nuclear receptor antagonist, a cytokine modulator, a chemokine modulator, and any combination thereof.

7. The method according to claim 1, wherein said blood sample is a blood sample not being inhibited from clotting by ethylenediaminetetraacetic acid (EDTA).

8. The method according to claim 1, wherein step (b) comprises a step (i) of comparing said kinase activity profile to a reference kinase activity profile, wherein said reference kinase activity profile is representative of a good or poor responder to said PD-1 or PD-L1 ICI; and a step (ii) of determining the response of said patient to treatment with said PD-1 or PD-L1 ICI on the basis of the comparison of said kinase activity profile with said reference kinase activity profile.

9. The method according to claim 1, wherein in step (a) said kinase activity is determined by contacting the blood sample with at least two of the protein kinase substrates as listed in Table 2 or Table 3, thereby providing a phosphorylation profile of said blood sample, said phosphorylation profile comprising the phosphorylation levels of phosphorylation sites present in said at least two protein kinase substrates.

10. The method according to claim 1, wherein said patient in need of an ICI is a patient diagnosed with a neoplastic disease, preferably wherein said neoplastic disease is selected from the group consisting of non-small-cell lung carcinoma (NSCLC), bladder cancer, ovarian cancer, prostate cancer, head and neck cancer, colorectal cancer, and melanoma.

11. Use of the method according to claim 1 for assessing the response of a patient in need of an ICI to treatment with a PD-1 or PD-L1 ICI.

12. Use of the method according to claim 1 for assessing the pharmaceutical or clinical value of a PD-1 or PD-L1 ICI.

13. A kit for predicting the response of a patient in need of an ICI to treatment with a PD-1 or PD-L1 ICI, according to claim 1, comprising means for measuring the kinase activity of at least two kinases independently selected from within at least two families of kinases selected from the group consisting of:
the VEGFR or PDGFR family of kinases;
the SRC family of kinases;
the SYK family of kinases;
the TAM family of kinases;
the JakA family of kinases; and
the ALK family of kinases,
in a blood sample obtained from said patient.

14. The kit according to claim 13, wherein the means for measuring the kinase activity at least two kinases independently selected from within at least two families of kinases selected from the group consisting of:
the VEGFR or PDGFR family of kinases;
the SRC family of kinases;
the SYK family of kinases;
the TAM family of kinases;
the JakA family of kinases; and
the ALK family of kinases,
are at least one array comprising all of the 142 peptide markers as listed in Table 2 or all of the 62 peptide markers as listed in Table 3.


SEQUENCE LISTING (143 sequences)

SEQ ID NO: 1 - protein, 13 aa, Artificial Sequence
  CD79A_181_193
EYEDENLYEGLNL

SEQ ID NO: 2 - protein, 13 aa, Artificial Sequence
  P85A_600_612
NENTEDQYSLVED

SEQ ID NO: 3 - protein, 13 aa, Artificial Sequence
  EPOR_419_431
ASAASFEYTILDP

SEQ ID NO: 4 - protein, 13 aa, Artificial Sequence
  PRRX2_202_214
WTASSPYSTVPPY

SEQ ID NO: 5 - protein, 15 aa, Artificial Sequence
  JAK2_563_577
VRREVGDYGQLHETE

SEQ ID NO: 6 - protein, 13 aa, Artificial Sequence
  CTNB1_79_91
VADIDGQYAMTRA

SEQ ID NO: 7 - protein, 13 aa, Artificial Sequence
  RON_1346_1358
SALLGDHYVQLPA

SEQ ID NO: 8 - protein, 13 aa, Artificial Sequence
  PGFRB_1002_1014
LDTSSVLYTAVQP

SEQ ID NO: 9 - protein, 13 aa, Artificial Sequence
  K2C6B_53_65
GAGFGSRSLYGLG

SEQ ID NO: 10 - protein, 13 aa, Artificial Sequence
  CD3Z_116_128
KDKMAEAYSEIGM

SEQ ID NO: 11 - protein, 13 aa, Artificial Sequence
  PGFRB_709_721
RPPSAELYSNALP

SEQ ID NO: 12 - protein, 13 aa, Artificial Sequence
  ZAP70_485_497
ALGADDSYYTARS

SEQ ID NO: 13 - protein, 13 aa, Artificial Sequence
  ENOG_37_49
SGASTGIYEALEL

SEQ ID NO: 14 - protein, 15 aa, Artificial Sequence
  PGFRB_1014_1028
PNEGDNDYIIPLPDP

SEQ ID NO: 15 - protein, 13 aa, Artificial Sequence
  PDPK1_369_381
DEDCYGNYDNLLS

SEQ ID NO: 16 - protein, 13 aa, Artificial Sequence
  PP2AB_297_309
EPHVTRRTPDYFL

SEQ ID NO: 17 - protein, 13 aa, Artificial Sequence
  PRGR_786_798
EQRMKESSFYSLC

SEQ ID NO: 18 - protein, 13 aa, Artificial Sequence
  MK12_178_190
ADSEMTGYVVTRW

SEQ ID NO: 19 - protein, 13 aa, Artificial Sequence
  ANXA2_17_29
HSTPPSAYGSVKA

SEQ ID NO: 20 - protein, 13 aa, Artificial Sequence
  VGFR2_1046_1058
DFGLARDIYKDPD

SEQ ID NO: 21 - protein, 13 aa, Artificial Sequence
  VGFR1_1040_1052
DFGLARDIYKNPD

SEQ ID NO: 22 - protein, 13 aa, Artificial Sequence
  CALM_95_107
KDGNGYISAAELR

SEQ ID NO: 23 - protein, 13 aa, Artificial Sequence
  EGFR_1103_1115
GSVQNPVYHNQPL

SEQ ID NO: 24 - protein, 13 aa, Artificial Sequence
  LCK_387_399
RLIEDNEYTAREG

SEQ ID NO: 25 - protein, 13 aa, Artificial Sequence
  SRC8_CHICK_492_504
YQAEENTYDEYEN

SEQ ID NO: 26 - protein, 13 aa, Artificial Sequence
  ANXA1_14_26
IENEEQEYVQTVK

SEQ ID NO: 27 - protein, 13 aa, Artificial Sequence
  FGFR2_762_774
TLTTNEEYLDLSQ

SEQ ID NO: 28 - protein, 13 aa, Artificial Sequence
  EGFR_862_874
LGAEEKEYHAEGG

SEQ ID NO: 29 - protein, 13 aa, Artificial Sequence
  C1R_199_211
TEASGYISSLEYP

SEQ ID NO: 30 - protein, 13 aa, Artificial Sequence
  EPHB1_921_933
SAIKMVQYRDSFL

SEQ ID NO: 31 - protein, 13 aa, Artificial Sequence
  VGFR2_944_956
RFRQGKDYVGAIP

SEQ ID NO: 32 - protein, 13 aa, Artificial Sequence
  EGFR_1118_1130
APSRDPHYQDPHS

SEQ ID NO: 33 - protein, 13 aa, Artificial Sequence
  EPHB4_583_595
IGHGTKVYIDPFT

SEQ ID NO: 34 - protein, 13 aa, Artificial Sequence
  PTN11_539_551
SKRKGHEYTNIKY

SEQ ID NO: 35 - protein, 13 aa, Artificial Sequence
  MK01_180_192
HTGFLTEYVATRW

SEQ ID NO: 36 - protein, 13 aa, Artificial Sequence
  MET_1227_1239
RDMYDKEYYSVHN

SEQ ID NO: 37 - protein, 13 aa, Artificial Sequence
  EFS_246_258
GGTDEGIYDVPLL

SEQ ID NO: 38 - protein, 13 aa, Artificial Sequence
  LAT_194_206
MESIDDYVNVPES

SEQ ID NO: 39 - protein, 13 aa, Artificial Sequence
  FGFR3_753_765
TVTSTDEYLDLSA

SEQ ID NO: 40 - protein, 13 aa, Artificial Sequence
  VGFR1_1320_1332_C1320S/C1321S
SSSPPPDYNSVVL

SEQ ID NO: 41 - protein, 13 aa, Artificial Sequence
  PLCG1_1246_1258
EGSFESRYQQPFE

SEQ ID NO: 42 - protein, 13 aa, Artificial Sequence
  PRGR_545_557
LRPDSEASQSPQY

SEQ ID NO: 43 - protein, 13 aa, Artificial Sequence
  CALM_93_105
FDKDGNGYISAAE

SEQ ID NO: 44 - protein, 13 aa, Artificial Sequence
  VGFR1_1235_1247
ATSMFDDYQGDSS

SEQ ID NO: 45 - protein, 13 aa, Artificial Sequence
  VGFR2_1207_1219_C1208S
VSDPKFHYDNTAG

SEQ ID NO: 46 - protein, 13 aa, Artificial Sequence
  RBL2_99_111
VPTVSKGTVEGNY

SEQ ID NO: 47 - protein, 13 aa, Artificial Sequence
  TNNT1_2_14
SDTEEQEYEEEQP

SEQ ID NO: 48 - protein, 13 aa, Artificial Sequence
  JAK1_1015_1027
AIETDKEYYTVKD

SEQ ID NO: 49 - protein, 13 aa, Artificial Sequence
  SRC8_CHICK_476_488
EYEPETVYEVAGA

SEQ ID NO: 50 - protein, 13 aa, Artificial Sequence
  VGFR1_1326_1338
DYNSVVLYSTPPI

SEQ ID NO: 51 - protein, 13 aa, Artificial Sequence
  EGFR_1165_1177
ISLDNPDYQQDFF

SEQ ID NO: 52 - protein, 13 aa, Artificial Sequence
  ODBA_340_352_D340K
KDSSAYRSVDEVN

SEQ ID NO: 53 - protein, 13 aa, Artificial Sequence
  EGFR_908_920
MTFGSKPYDGIPA

SEQ ID NO: 54 - protein, 13 aa, Artificial Sequence
  VGFR1_1046_1058_Y1048F
DIFKNPDYVRKGD

SEQ ID NO: 55 - protein, 13 aa, Artificial Sequence
  EGFR_1062_1074
EDSFLQRYSSDPT

SEQ ID NO: 56 - protein, 13 aa, Artificial Sequence
  DDR1_506_518
LLLSNPAYRLLLA

SEQ ID NO: 57 - protein, 13 aa, Artificial Sequence
  AMPE_5_17
EREGSKRYCIQTK

SEQ ID NO: 58 - protein, 13 aa, Artificial Sequence
  EPHA4_589_601
LNQGVRTYVDPFT

SEQ ID NO: 59 - protein, 13 aa, Artificial Sequence
  DYR1A_312_324
CQLGQRIYQYIQS

SEQ ID NO: 60 - protein, 13 aa, Artificial Sequence
  EPOR_361_373
SEHAQDTYLVLDK

SEQ ID NO: 61 - protein, 13 aa, Artificial Sequence
  PLCG1_764_776
IGTAEPDYGALYE

SEQ ID NO: 62 - protein, 13 aa, Artificial Sequence
  TEC_512_524
RYFLDDQYTSSSG

SEQ ID NO: 63 - protein, 13 aa, Artificial Sequence
  ERBB4_1277_1289
IVAENPEYLSEFS

SEQ ID NO: 64 - protein, 13 aa, Artificial Sequence
  PLCG1_776_788
EGRNPGFYVEANP

SEQ ID NO: 65 - protein, 13 aa, Artificial Sequence
  FABPH_13_25
DSKNFDDYMKSLG

SEQ ID NO: 66 - protein, 13 aa, Artificial Sequence
  PERI_458_470
QRSELDKSSAHSY

SEQ ID NO: 67 - protein, 13 aa, Artificial Sequence
  STAT4_686_698
TERGDKGYVPSVF

SEQ ID NO: 68 - protein, 13 aa, Artificial Sequence
  DYR1A_212_224
KHDTEMKYYIVHL

SEQ ID NO: 69 - protein, 13 aa, Artificial Sequence
  ERBB4_1181_1193
QALDNPEYHNASN

SEQ ID NO: 70 - protein, 13 aa, Artificial Sequence
  TYRO3_679_691
KIYSGDYYRQGCA

SEQ ID NO: 71 - protein, 13 aa, Artificial Sequence
  FAK2_572_584
RYIEDEDYYKASV

SEQ ID NO: 72 - protein, 13 aa, Artificial Sequence
  PAXI_111_123
VGEEEHVYSFPNK

SEQ ID NO: 73 - protein, 13 aa, Artificial Sequence
  ERBB2_1241_1253
PTAENPEYLGLDV

SEQ ID NO: 74 - protein, 13 aa, Artificial Sequence
  EGFR_1190_1202
STAENAEYLRVAP

SEQ ID NO: 75 - protein, 13 aa, Artificial Sequence
  MBP_259_271
FGYGGRASDYKSA

SEQ ID NO: 76 - protein, 13 aa, Artificial Sequence
  EPHA2_581_593
QLKPLKTYVDPHT

SEQ ID NO: 77 - protein, 13 aa, Artificial Sequence
  STAT1_694_706
DGPKGTGYIKTEL

SEQ ID NO: 78 - protein, 13 aa, Artificial Sequence
  MK01_198_210
IMLNSKGYTKSID

SEQ ID NO: 79 - protein, 13 aa, Artificial Sequence
  STA5A_687_699
LAKAVDGYVKPQI

SEQ ID NO: 80 - protein, 13 aa, Artificial Sequence
  VGFR1_1049_1061
KNPDYVRKGDTRL

SEQ ID NO: 81 - protein, 13 aa, Artificial Sequence
  B3AT_39_51
TEATATDYHTTSH

SEQ ID NO: 82 - protein, 13 aa, Artificial Sequence
  FGFR1_761_773
TSNQEYLDLSMPL

SEQ ID NO: 83 - protein, 13 aa, Artificial Sequence
  RAF1_332_344
PRGQRDSSYYWEI

SEQ ID NO: 84 - protein, 13 aa, Artificial Sequence
  FRK_380_392
KVDNEDIYESRHE

SEQ ID NO: 85 - protein, 13 aa, Artificial Sequence
  VGFR2_1168_1180
AQQDGKDYIVLPI

SEQ ID NO: 86 - protein, 13 aa, Artificial Sequence
  CDK7_157_169
GLAKSFGSPNRAY

SEQ ID NO: 87 - protein, 13 aa, Artificial Sequence
  VGFR1_1162_1174
VQQDGKDYIPINA

SEQ ID NO: 88 - protein, 13 aa, Artificial Sequence
  NTRK2_509_521
PVIENPQYFGITN

SEQ ID NO: 89 - protein, 13 aa, Artificial Sequence
  STAT6_634_646
MGKDGRGYVPATI

SEQ ID NO: 90 - protein, 13 aa, Artificial Sequence
  EPHA4_921_933
QAIKMDRYKDNFT

SEQ ID NO: 91 - protein, 13 aa, Artificial Sequence
  RET_680_692
AQAFPVSYSSSGA

SEQ ID NO: 92 - protein, 13 aa, Artificial Sequence
  MBP_263_275
GRASDYKSAHKGF

SEQ ID NO: 93 - protein, 13 aa, Artificial Sequence
  ACHD_383_395
YISKAEEYFLLKS

SEQ ID NO: 94 - protein, 13 aa, Artificial Sequence
  KSYK_518_530
ALRADENYYKAQT

SEQ ID NO: 95 - protein, 13 aa, Artificial Sequence
  CBL_693_705
EGEEDTEYMTPSS

SEQ ID NO: 96 - protein, 12 aa, Artificial Sequence
  ART_004_EAIYAAPFAKKKXC
EAIYAAPFAKKK

SEQ ID NO: 97 - protein, 13 aa, Artificial Sequence
  VGFR2_1052_1064
DIYKDPDYVRKGD

SEQ ID NO: 98 - protein, 13 aa, Artificial Sequence
  NPT2A_501_513
AKALGKRTAKYRW

SEQ ID NO: 99 - protein, 13 aa, Artificial Sequence
  VGFR1_1206_1218
GSSDDVRYVNAFK

SEQ ID NO: 100 - protein, 13 aa, Artificial Sequence
  STAT3_698_710
DPGSAAPYLKTKF

SEQ ID NO: 101 - protein, 13 aa, Artificial Sequence
  INSR_1348_1360
SLGFKRSYEEHIP

SEQ ID NO: 102 - protein, 13 aa, Artificial Sequence
  FGFR3_641_653
DVHNLDYYKKTTN

SEQ ID NO: 103 - protein, 13 aa, Artificial Sequence
  NTRK1_489_501
HIIENPQYFSDAC

SEQ ID NO: 104 - protein, 13 aa, Artificial Sequence
  ODPAT_291_303
SMSDPGVSYRTRE

SEQ ID NO: 105 - protein, 13 aa, Artificial Sequence
  SRC8_CHICK_470_482
VSQREAEYEPETV

SEQ ID NO: 106 - protein, 13 aa, Artificial Sequence
  RON_1353_1365
YVQLPATYMNLGP

SEQ ID NO: 107 - protein, 13 aa, Artificial Sequence
  LAT_249_261
EEGAPDYENLQEL

SEQ ID NO: 108 - protein, 13 aa, Artificial Sequence
  CDK2_8_20
EKIGEGTYGVVYK

SEQ ID NO: 109 - protein, 13 aa, Artificial Sequence
  NTRK2_696_708
GMSRDVYSTDYYR

SEQ ID NO: 110 - protein, 13 aa, Artificial Sequence
  CRK_214_226
GPPEPGPYAQPSV

SEQ ID NO: 111 - protein, 13 aa, Artificial Sequence
  VGFR3_1061_1073
DIYKDPDYVRKGS

SEQ ID NO: 112 - protein, 13 aa, Artificial Sequence
  NCF1_313_325
QRSRKRLSQDAYR

SEQ ID NO: 113 - protein, 13 aa, Artificial Sequence
  MK07_211_223
AEHQYFMTEYVAT

SEQ ID NO: 114 - protein, 13 aa, Artificial Sequence
  INSR_992_1004
YASSNPEYLSASD

SEQ ID NO: 115 - protein, 13 aa, Artificial Sequence
  ZBT16_621_633
LRTHNGASPYQCT

SEQ ID NO: 116 - protein, 13 aa, Artificial Sequence
  MK14_173_185
RHTDDEMTGYVAT

SEQ ID NO: 117 - protein, 13 aa, Artificial Sequence
  VINC_815_827
KSFLDSGYRILGA

SEQ ID NO: 118 - protein, 13 aa, Artificial Sequence
  STAT4_714_726
PSDLLPMSPSVYA

SEQ ID NO: 119 - protein, 13 aa, Artificial Sequence
  FER_707_719
RQEDGGVYSSSGL

SEQ ID NO: 120 - protein, 13 aa, Artificial Sequence
  EPHA2_765_777
EDDPEATYTTSGG

SEQ ID NO: 121 - protein, 13 aa, Artificial Sequence
  ERBB2_870_882
LDIDETEYHADGG

SEQ ID NO: 122 - protein, 13 aa, Artificial Sequence
  FAK1_569_581
RYMEDSTYYKASK

SEQ ID NO: 123 - protein, 13 aa, Artificial Sequence
  PGFRB_771_783
YMAPYDNYVPSAP

SEQ ID NO: 124 - protein, 13 aa, Artificial Sequence
  DCX_109_121
GIVYAVSSDRFRS

SEQ ID NO: 125 - protein, 13 aa, Artificial Sequence
  PGFRB_768_780
SSNYMAPYDNYVP

SEQ ID NO: 126 - protein, 13 aa, Artificial Sequence
  MK10_216_228
TSFMMTPYVVTRY

SEQ ID NO: 127 - protein, 13 aa, Artificial Sequence
  RB_804_816
IYISPLKSPYKIS

SEQ ID NO: 128 - protein, 13 aa, Artificial Sequence
  MBP_198_210
ARTAHYGSLPQKS

SEQ ID NO: 129 - protein, 13 aa, Artificial Sequence
  K2C8_425_437
SAYGGLTSPGLSY

SEQ ID NO: 130 - protein, 13 aa, Artificial Sequence
  CD3Z_146_158
STATKDTYDALHM

SEQ ID NO: 131 - protein, 13 aa, Artificial Sequence
  EPHB1_771_783
DDTSDPTYTSSLG

SEQ ID NO: 132 - protein, 13 aa, Artificial Sequence
  PAXI_24_36
FLSEETPYSYPTG

SEQ ID NO: 133 - protein, 13 aa, Artificial Sequence
  RET_1022_1034
TPSDSLIYDDGLS

SEQ ID NO: 134 - protein, 13 aa, Artificial Sequence
  PECA1_706_718
KKDTETVYSEVRK

SEQ ID NO: 135 - protein, 13 aa, Artificial Sequence
  FES_706_718
REEADGVYAASGG

SEQ ID NO: 136 - protein, 13 aa, Artificial Sequence
  VGFR2_989_1001
EEAPEDLYKDFLT

SEQ ID NO: 137 - protein, 13 aa, Artificial Sequence
  PGFRB_572_584
VSSDGHEYIYVDP

SEQ ID NO: 138 - protein, 13 aa, Artificial Sequence
  EPHA1_774_786
LDDFDGTYETQGG

SEQ ID NO: 139 - protein, 13 aa, Artificial Sequence
  41_654_666
LDGENIYIRHSNL

SEQ ID NO: 140 - protein, 13 aa, Artificial Sequence
  RASA1_453_465
TVDGKEIYNTIRR

SEQ ID NO: 141 - protein, 13 aa, Artificial Sequence
  PDPK1_2_14
ARTTSQLYDAVPI

SEQ ID NO: 142 - protein, 13 aa, Artificial Sequence
  EPHA7_607_619
TYIDPETYEDPNR

SEQ ID NO: 143 - protein, 7 aa, Artificial Sequence
  TAM family conserved kinase domain sequence
  SITE (3)..(3): wherein X can be I or L
  SITE (5)..(5): wherein X can be I or L
KWXAXES